(19) United States
(12) Patent Application Publication
(10) Pub. No.: US 2025/0354219 A1
(43) Pub. Date: Nov. 20, 2025

(54) METHODS FOR NEUROENDOCRINE CANCER DETECTION IN SALIVA

(71) Applicant: Liquid Biopsy Research LLC, Charlestown (KN)

(72) Inventors: Irvin Mark MODLIN, Woodbridge, CT (US); Mark KIDD, New Haven, CT (US); Ignat DROZDOV, Stratford Upon Avon (GB)

(21) Appl. No.: 19/117,177

(22) PCT Filed: Sep. 29, 2023
(86) PCT No.: PCT/US2023/075498

§ 371 Date: Mar. 31, 2025

Related U.S. Application Data
(60) Provisional application No. 63/377,808, filed on Sep. 30, 2022.

(51) Int. Cl. C12Q 1/6886; G16B 40/20

(52) U.S. Cl. CPC C12Q 1/6886; G16B 40/20; C12Q 2600/158

(57) ABSTRACT

The present inventions are directed to methods for detecting a neuroendocrine cancer in saliva, methods for determining the completeness of surgery, methods for determining whether a neuroendocrine cancer is stable or progressive, and methods for evaluating the response to a neuroendocrine cancer therapy.

### RELATED APPLICATIONS

This application claims priority to, and the benefit of, U.S. Provisional Application No. 63/377,808, filed Sep. 30, 2022, the contents of which are incorporated herein by reference in their entireties.

### REFERENCE TO ELECTRONIC SEQUENCE LISTING

The contents of the electronic sequence listing (LBIO-007_001WO_SeqList_ST26.xml; Size: 207,502 bytes; and Date of Creation: Sep. 27, 2023) are herein incorporated by reference in their entirety.

### BACKGROUND

Neuroendocrine cancers, also referred to as neuroendocrine neoplasms (NENs) or neuroendocrine tumors (NETs), are tumors that derive from specialized cells of the body's neuroendocrine system. These cells have traits of both hormone-producing endocrine cells and nerve cells. They are found throughout the body's organs including the gastrointestinal (GI) tract, the pancreas and the lung but can also occur in other sites like adrenal glands (pheochromocytomas) or the central nervous system (paragangliomas) or the pituitary gland. The incidence and prevalence of NET/NEN have increased between 100 and 600 percent in the U.S. over the last thirty years, with no significant increase in survival. Symptoms of neuroendocrine cancers include skin flushing and sweating, wheezing, coughing and difficulty breathing, diarrhea, coughing, sweating, weight gain, pain or other areas from cancer that has spread to bones, and rapid heartbeat and marked changes in blood pressure.

Heterogeneity and complexity of these tumors has made diagnosis, treatment, and classification difficult. These neoplasms lack several mutations commonly associated with other cancers and microsatellite instability is largely absent. Individual histopathologic subtypes as determined from tissue resources e.g., biopsy, can be associated with distinct clinical behavior, but there is no definitive, generally accepted molecular pathologic classification or prediction scheme, hindering diagnosis, staging, treatment assessment and follow-up.

Existing diagnostic and prognostic approaches for tumors include imaging (e.g., CT or MRI), histology, measurements of circulating hormones and proteins e.g., chromogranin A and detection of some gene products. Available methods are limited, for example, by low sensitivity and/or specificity, the inability to detect early-stage disease, and the ongoing exposure to radiation risk associated with imaging protocols. Tumors often go undiagnosed until they are metastatic and often untreatable. In addition, follow-up is difficult, particularly in patients with residual disease burden.

Molecular genetic information is being used to understand neuroendocrine cancer biology but there remains an incomplete understanding of the molecular mechanisms underpinning pathogenesis and an absence of molecular-based biomarkers that can be used to predict sensitivity to therapeutic agents. Consequently, the development of diagnostic methods that more accurately define disease status, identify sensitivity to therapy and can ultimately be used to better monitor disease progression, is critical.

Surveillance remains a cornerstone approach to monitor neuroendocrine cancers and detect recurrence at an early stage. After potentially curative resection, monitoring can be undertaken through measurement of blood biomarkers and/or imaging like CT to detect asymptomatic metastatic disease early.

The current biomarker used for monitoring is Chromogranin A (CgA). The sensitivity and specificity of this marker is poor and other hormone markers specific to the primary tumor may be used. Irrespective, detecting residual disease remains difficult and protocols typically engender significant patient/physician concern.

Tissue grading, although used to identify the timing of imaging, has similarly proven to have a low sensitivity and specificity for predicting recurrence.

Saliva is an important testing compartment that allows evaluation of biomarkers for viral, bacterial, and fungal parasitic infections as well as for the measurement of markers that characterize systemic and non-systemic disease. Human RNA obtained from cell-free saliva has been evaluated using sequencing and PCR technologies. Cell-free RNA from healthy individuals contains more than 3,000 species of mRNA. RNA typically enters the oral cavity through secretion (from the parotid, submandibular and sublingual glands) as a component of gingival crevice fluid and from desquamated oral epithelial cells. RNA can originate form acinar cells or by circulation.

Saliva has been determined as a testing compartment for other cancers e.g., head and neck tumors. Typically, viral DNA (HPV) is isolated and amplified. This is used to provide a diagnosis of the disease. Recently, tumor RNA has been detected in saliva. For example, a 4 gene RNA based biomarker was developed for the diagnosis of oral cancer. The source of RNA may be from the salivary glands themselves or be secondary to cells e.g., lymphocytes, that are secreted into the mouth. It is also known that salivary glands are vascularized and filter blood products. This suggests that blood may also be a source of RNA detectable in saliva.

### SUMMARY

The present disclosure provides a method of identifying the presence or absence of a neuroendocrine cancer in a subject in need thereof, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying the presence of the neuroendocrine cancer in the subject when the score is greater than or equal to the predetermined cutoff value or determining the absence of the neuroendocrine cancer in the subject when the score is less than the predetermined cutoff value. In some aspects, the predetermined cutoff value is 26% on a scale of 0-100%.

The present disclosure provides a method of determining whether a neuroendocrine cancer in a subject is stable or progressive, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) determining that the neuroendocrine cancer is progressive when the score is greater than or equal to the predetermined cutoff value or determining that the neuroendocrine cancer is stable when the score is less than the predetermined cutoff value. In some aspects, the predetermined cutoff value is 50% on a scale of 0 to 100%.

The present disclosure provides a method of determining the completeness of surgery to remove a neuroendocrine cancer in a subject, the method comprising: (a) determining the expression level of at least 36 biomarkers in a test sample from the subject after the surgery, wherein the 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying that the neuroendocrine cancer is not completely removed when the score is greater than or equal to the predetermined cutoff value or identifying that the neuroendocrine cancer is completely removed based when the score is less than the predetermined cutoff value. In some aspects the predetermined cutoff value is 50% on a scale of 0 to 100%.

The present disclosure provides a method of evaluating the response of a subject having a neuroendocrine cancer to an anti-neuroendocrine cancer therapy, the method comprising: (a) at a first time point: (i) determining the expression level of at least 36 biomarkers in a test sample from the subject, wherein the at least 38 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (iii) inputting each normalized expression level from step (a)(ii) into an algorithm to generate a first score; (b) at a second time point, wherein the second time point is after the first time point and after the administration of the anti-neuroendocrine therapy to the subject: (i) determining the expression level of the at least 36 biomarkers in a test sample from the subject; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT12, VPS13C, WDFY3, and ZXDC; and (iii) inputting each normalized expression level from step (b)(ii) into an algorithm to generate a second score; (c) comparing the first score and the second score; and (d) identifying that the subject is responsive to the anti-neuroendocrine cancer therapy when the second score is decreased as compared to the first score or identifying that the subject is not responsive to the anti-neuroendocrine cancer therapy when the second score is not decreased as compared to the first score. In some aspects, the subject is identified as responsive to the anti-neuroendocrine cancer therapy when the second is at least 5% less than the first score.

In some aspects of the preceding methods, the housekeeping gene is selected from the group consisting of ATG4B, RHOA, TOX4, TPT1, and TXNIP.

In some aspects of the preceding methods, the housekeeping gene is RHOA.

In some aspects of the preceding methods, the method has a sensitivity of at least 90%.

In some aspects of the preceding methods, the method has a specificity of at least 90%.

In some aspects of the preceding methods, at least one of the at least 36 biomarkers is RNA, cDNA, or protein.

In some aspects of the preceding methods, wherein when the biomarker is RNA, the RNA is reverse transcribed to produce cDNA, and the produced cDNA expression level is detected.

In some aspects of the preceding methods, the expression level of the biomarker is detected by forming a complex between the biomarker and a labeled probe or primer.

In some aspects of the preceding methods, wherein when the biomarker is protein, the protein detected by forming a complex between the protein and a labeled antibody. In some aspects, the label is a fluorescent label.

In some aspects of the preceding methods, wherein when the biomarker is RNA or cDNA, the RNA or cDNA is detected by forming a complex between the RNA or cDNA and a labeled nucleic acid probe or primer. In some aspects, the label is a fluorescent label. In some aspects, wherein the complex between the RNA or cDNA and the labeled nucleic acid probe or primer is a hybridization complex.

In some aspects of the preceding methods, the first predetermined cutoff value is derived from a plurality of reference samples obtained from subjects not having or not diagnosed with a neoplastic disease. In some aspects, the neoplastic disease is neuroendocrine cancer.

In some aspects of the preceding methods, the algorithm is XGB, RF, glmnet, cforest, CART, treebag, knn, nnet, SVM-radial, SVM-linear, NB, or mlp. In some aspects the algorithm is RF, preferably wherein the RF algorithm is a grid-search optimized Random-Forest.

In some aspects of the preceding methods, the machine learning algorithm is trained using the expression levels or normalized expression levels of the at least 36 biomarkers obtained from a plurality of reference samples obtained from subjects not having a neuroendocrine cancer and the expression levels or normalized expression levels of the at least 36 biomarkers from a plurality of reference samples obtained from subjects having neuroendocrine cancer.

In some aspects of the preceding methods, the methods further comprise treating the subject identified as having a neuroendocrine cancer with at least one anti-neuroendocrine cancer therapy.

In some aspects of the preceding methods, the anti-neuroendocrine cancer therapy comprises active surveillance, surgery, cryotherapy, chemotherapy, targeted treatment, radiation therapy, or any combination thereof.

In some aspects of the preceding methods, the targeted therapy comprises somatostatin analogue therapy, everolimus, sunitinib, immunotherapy or any combination thereof.

In some aspects of the preceding methods, the chemotherapy comprises capecitabine, temozolomide, or any combination thereof.

In some aspects of the preceding methods, the radiation treatment comprises peptide receptor radionuclide therapy (PRRT).

In some aspects of the preceding methods, the first time point is prior to the administration of the therapy to the subject.

In some aspects of the preceding methods, the first time point is after the administration of the therapy to the subject.

In some aspects of the preceding methods, the test sample is saliva.

In some aspects of the preceding methods, the test sample is self-collected saliva into a container with stabilization fluid.

### BRIEF DESCRIPTION OF THE DRAWINGS

FIG. 1 is a graph showing the relationship between gene expression in blood and in saliva.

FIG. 2A and FIG. 2B are X-Y scatter graphs showing the concordance between Ct values in blood and saliva (FIG. 2A) and normalized gene expression in blood and saliva (FIG. 2B). Red line is the linear correlation. Vertical and horizontal lines SEM and SD, respectively of the averaged values from the 36 target genes.

FIG. 3 is a graph showing the relationship between normalized gene expression in tumour samples and in saliva. Red line is the linear correlation. Vertical and horizontal lines SEM and SD, respectively of the averaged values from the 36 target genes.

FIG. 4 is a graph showing gene expression in age/sex-matched controls: n=30, and neuroendocrine cancer cases: n=15). Expression levels were significantly (p<0.05) elevated in 28 genes and was significantly decreased in 8 of the target genes.

FIG. 5A, FIG. 5B and FIG. 5C are graphs showing visualisation of 36 putative marker genes identified by the Random Forest algorithm in the derivation cohort of n=274 control samples and 76 cancer samples. (FIG. 5A) Expression normalized to ATG4B. (FIG. 5B) Expression normalized to RHOA. (FIG. 5C) Expression normalized to TXNIP.

FIG. 6 is a graph showing NETsaliva score in an independent set of controls (n=108) and neuroendocrine cancers (n=30). Levels were significantly elevated (p<0.0001) in NETs (57±15) versus controls (15±11).

FIG. 7 is a graph showing receiver operator curve analysis of the testing partition in the independent set. The AUROC was 0.98. The Youden J index was 0.88. The Z-statistic was highly significant (54.9; p<0.0001).

FIG. 8 is a graph showing the metrics of the assay for determining neuroendocrine cancers. The sensitivity was 100% and specificity was 88%.

FIG. 9 is a graph showing the effect of surgery on the NETsaliva score. Levels prior to surgery are elevated (66±10%). Surgery reduced levels to 30±12% (p<0.0001), not different to control levels.

FIG. 10A-B are spider plot graphs showing the effect of treatment on the NETsaliva score. Levels prior to treatment are elevated (63±44%). In those who responded to therapy, levels were reduced by −40±31% and −51±25% at the two follow-up time points (p<0.0001). In those who progressed despite therapy, levels were increased by +7±16% and +32±30% (p<0.05), respectively. (FIG. 10A) Spider plot of all patients. (FIG. 10B) Spider plot of individual responders (blue) and those who progress (red).

### DETAILED DESCRIPTION

The details of the inventions are set forth in the accompanying description below. Although methods and materials similar or equivalent to those described herein can be used in the practice or testing of the present inventions, illustrative methods and materials are now described. Other features, objects, and advantages of the inventions will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which these inventions belongs. All patents and publications cited in this specification are incorporated herein by reference in their entireties.

Described herein are methods to quantitate (score) a salivary neuroendocrine cancer molecular signature with high sensitivity and specificity for purposes including, but not limited to, detecting a NET/NEN, determining whether a NET/NEN is stable or progressive, determining the completeness of surgery, evaluating the response of a subject to a neuroendocrine cancer therapy, treating a NET/NEN in a subject, or any combination thereof. Without wishing to be bound by theory, the present inventions are based on the discovery that the expression levels of AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC, normalized by the expression level of a housekeeping gene, are elevated in subjects having neuroendocrine cancers as compared to healthy subjects.

As described herein, measurements of the expression level of the circulating neuroendocrine cancer transcripts described above (referred to collectively as the “NETsaliva transcripts”) in a saliva sample from a subject can be used to diagnose neuroendocrine cancer. In non-limiting examples, the expression levels of the NETsaliva transcripts as measured from a saliva sample from a subject can be inputted into an algorithm to generate a score (referred to herein as a “NETsaliva score”), which can be used to diagnose the presence of a NET/NEN in a subject. Moreover, decreases in a subject's NETsaliva score after administration of one or more anti-neuroendocrine cancer therapies (e.g. surgery and chemotherapy) can be used to determine the subject's responsiveness to that one or more therapies, optionally in combination with standard clinical assessment and imaging.

Accordingly, the present disclosure provides methods of identifying the presence or absence of a neuroendocrine cancer in a subject in need thereof, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) identifying the presence or absence of the neuroendocrine cancer in the subject based on the normalized expression levels from step (b). In some aspects, identifying the presence of absence of the neuroendocrine cancer in the subject based on the normalized expression levels from step (b) can comprise comparing the normalized expression levels to corresponding predetermined cutoff values and identifying the presence or absence of the neuroendocrine cancer in the subject based on the relationship between the normalized expression levels and the corresponding predetermined cutoff values (e.g. greater than, greater than or equal to, less than, less than or equal to, or equal to).

The present disclosure provides methods of identifying the presence or absence of a neuroendocrine cancer in a subject in need thereof, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; and (d) identifying the presence or absence of the neuroendocrine cancer in the subject based on the score. In some aspects, identifying the presence of absence of the neuroendocrine cancer in the subject based on the score can comprise comparing the score to a predetermined cutoff value and identifying the presence or absence of the neuroendocrine cancer in the subject based on the relationship between the score and the predetermined cutoff value (e.g. greater than, greater than or equal to, less than, less than or equal to, or equal to).

Accordingly, the present disclosure provides methods of identifying the presence or absence of a neuroendocrine cancer in a subject in need thereof, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying the presence of the neuroendocrine cancer in the subject when the score is greater than or equal to the predetermined cutoff value or determining the absence of the neuroendocrine cancer in the subject when the score is less than the predetermined cutoff value.

Accordingly, the present disclosure provides methods of identifying the presence or absence of a neuroendocrine cancer in a subject in need thereof, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying the presence of the neuroendocrine cancer in the subject when the score is greater than the predetermined cutoff value or determining the absence of the neuroendocrine cancer in the subject when the score is less than or equal to the predetermined cutoff value.

In some aspects of the preceding methods, the predetermined cutoff value can be 26% on a scale of 0-100%.

Accordingly, the present disclosure provides methods of identifying the risk of a subject having a neuroendocrine cancer, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) identifying the risk of the subject having a neuroendocrine cancer based on the normalized expression levels from step (b). In some aspects, identifying the risk of the subject having a neuroendocrine cancer based on the normalized expression levels from step (b) can comprise comparing the normalized expression levels to corresponding predetermined cutoff values and identifying the risk of the subject having a neuroendocrine cancer based on the relationship between the normalized expression levels and the corresponding predetermined cutoff values (e.g. greater than, greater than or equal to, less than, less than or equal to, or equal to).

The present disclosure provides methods of identifying the risk of a subject having a neuroendocrine cancer, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) inputting each normalized expression level from step (b) into an algorithm to generate a score; and (d) identifying the risk of the subject having a neuroendocrine cancer based on the score. In some aspects, identifying the risk of the subject having a neuroendocrine cancer based on the score can comprise comparing the score to a predetermined cutoff value and the risk of the subject having a neuroendocrine cancer based on the relationship between the score and the predetermined cutoff value (e.g. greater than, greater than or equal to, less than, less than or equal to, or equal to).

Accordingly, the present disclosure provides methods of identifying the risk of a subject having a neuroendocrine cancer, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying that the subject is at high risk of having a neuroendocrine cancer when the score is greater than or equal to the predetermined cutoff value or determining that the subject is at a low risk of having a neuroendocrine cancer when the score is less than the predetermined cutoff value.

Accordingly, the present disclosure provides methods of identifying the risk of a subject having a neuroendocrine cancer, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying that the subject is at high risk of having a neuroendocrine cancer when the score is greater than the predetermined cutoff value or determining that the subject is at a low risk of having a neuroendocrine cancer when the score is less than or equal to the predetermined cutoff value.

In some aspects of the preceding methods, the predetermined cutoff value can be 26% on a scale of 0-100%.

The present disclosure provides methods of determining whether a neuroendocrine cancer in a subject is stable or progressive, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) determining whether the neuroendocrine cancer in the subject is stable or progressive based on the normalized expression levels from step (b). In some aspects, determining whether the neuroendocrine cancer in the subject is stable or progressive based on the normalized expression levels from step (b) comprises comparing the normalized expression levels to corresponding predetermined cutoff values and determining whether the neuroendocrine cancer in the subject is stable or progressive based on the relationship between the normalized expression levels and the corresponding predetermined cutoff values (e.g. greater than, greater than or equal to, less than, less than or equal to, or equal to).

The present disclosure provides methods of determining whether a neuroendocrine cancer in a subject is stable or progressive, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) inputting each normalized expression level from step (b) into an algorithm to generate a score; and (d) determining whether the neuroendocrine cancer in the subject is stable or progressive based on the score. In some aspects, determining whether the neuroendocrine cancer in the subject is stable or progressive based on the score comprises comparing the score to a corresponding predetermined cutoff value and determining whether the neuroendocrine cancer in the subject is stable or progressive based on the relationship between the score and the predetermined cutoff value (e.g. greater than, greater than or equal to, less than, less than or equal to, or equal to).

Accordingly, the present disclosure provides methods of determining whether a neuroendocrine cancer in a subject is stable or progressive, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) determining that the neuroendocrine cancer is progressive when the score is greater than or equal to the predetermined cutoff value or determining that the neuroendocrine cancer is stable when the score is less than the predetermined cutoff value.

Accordingly, the present disclosure provides methods of determining whether a neuroendocrine cancer in a subject is stable or progressive, the method comprising: (a) determining the expression level of at least 36 biomarkers in a test sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) determining that the neuroendocrine cancer is progressive when the score is greater than the predetermined cutoff value or determining that the neuroendocrine cancer is stable when the score is less than or equal to the predetermined cutoff value.

In some aspects of the preceding methods, the predetermined cutoff value can be 50% on a scale of 0 to 100%.

Additionally, the present disclosure provides methods of determining the completeness of surgery to remove a neuroendocrine cancer in a subject, the method comprising: (a) determining the expression level of at least 36 biomarkers in a test sample from the subject after the surgery, wherein the 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (c) identifying that the neuroendocrine cancer is not completely removed or identifying that the neuroendocrine cancer is completely removed based on the normalized expression levels from step (b). In some aspects, identifying that the neuroendocrine cancer is not completely removed or identifying that the neuroendocrine cancer is completely removed based on the normalized expression levels from step (b) can comprise comparing the normalized expression levels to corresponding predetermined cutoff values and identifying that the neuroendocrine cancer is not completely removed or identifying that the neuroendocrine cancer is completely removed based on the relationship between the normalized expression levels and the corresponding predetermined cutoff values (e.g. greater than, greater than or equal to, less than, less than or equal to, or equal to).

Accordingly, the present disclosure provides methods of determining the completeness of surgery to remove a neuroendocrine cancer in a subject, the method comprising: (a) determining the expression level of at least 36 biomarkers in a test sample from the subject after the surgery, wherein the 36 biomarkers comprise AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; and (d) identifying that the neuroendocrine cancer is not completely removed or identifying that the neuroendocrine cancer is completely removed based on the score. In some aspects, identifying that the neuroendocrine cancer is not completely removed or identifying that the neuroendocrine cancer is completely removed based on the score can comprise comparing the score to a corresponding predetermined cutoff value and identifying that the neuroendocrine cancer is not completely removed or identifying that the neuroendocrine cancer is completely removed based on the relationship between the score and the predetermined cutoff value (e.g. greater than, greater than or equal to, less than, less than or equal to, or equal to).

Accordingly, the present disclosure provides methods of determining the completeness of surgery to remove a neuroendocrine cancer in a subject, the method comprising: (a) determining the expression level of at least 36 biomarkers in a test sample from the subject after the surgery, wherein the 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying that the neuroendocrine cancer is not completely removed when the score is greater than or equal to the predetermined cutoff value or identifying that the neuroendocrine cancer is completely removed based when the score is less than the predetermined cutoff value.

Accordingly, the present disclosure provides methods of determining the completeness of surgery to remove a neuroendocrine cancer in a subject, the method comprising: (a) determining the expression level of at least 36 biomarkers in a test sample from the subject after the surgery, wherein the 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT12, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) inputting each normalized expression level from step (b) into an algorithm to generate a score; (d) comparing the score to a predetermined cutoff value; and (e) identifying that the neuroendocrine cancer is not completely removed when the score is greater than the predetermined cutoff value or identifying that the neuroendocrine cancer is completely removed based when the score is less than or equal to the predetermined cutoff value.

In some aspects of the preceding methods, a predetermined cutoff value can be 50% on a scale of 0-100%.

The response of a subject having a neuroendocrine cancer to a therapy can also be evaluated by comparing the scores determined by the same algorithm at different time points of the therapy. For example, the first time point can be prior to or after the administration of the therapy to the subject; the second time point is after the first time point and after the administration of the therapy to the subject. A first score is generated at the first time point, and a second score is generated at the second time point. When the second score is decreased as compared to the first score, the subject is considered to be responsive to the therapy. In some aspects, the second score is decreased as compared to the first score when the second score is at least 5% less than the first score, e.g., at least 10% less than the first score, at least 15% less than the first score, at least 25% less than the first score, at least 40% less than the first score, at least 50% less than the first score, at least 75% less than the first score, or at least 90% less than the first score. When the second score is not significantly decreased or has increased as compared to the first score, the subject is considered to be not responsive to the therapy.

The present disclosure also provides methods of evaluating the response of a subject having a neuroendocrine cancer to an anti-neuroendocrine cancer therapy, the method comprising: (a) at a first time point: (i) determining the expression level of at least 36 biomarkers in a test sample from the subject, wherein the at least 38 biomarkers comprise AKAP8L, APLP2, ARAF, BNIP3L, BRAF, CD59, COMMD9, CTGF, FAM131A, FLJ103571ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (b) at a second time point, wherein the second time point is after the first time point and after the administration of the anti-neuroendocrine therapy to the subject: (i) determining the expression level of the at least 36 biomarkers in a test sample from the subject; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; (c) comparing the normalized expression levels from step (a)(ii) and step (b)(ii); and (d) identifying that the subject is responsive to the anti-neuroendocrine cancer therapy when the normalized expression levels from step (b)(ii) are decreased as compared to the expression levels from step (a)(ii) or identifying that the subject is not responsive to the anti-neuroendocrine cancer therapy when the normalized expression levels from step (b)(ii) are not decreased as compared to the normalized expression levels from step (a)(ii).

The present disclosure also provides methods of evaluating the response of a subject having a neuroendocrine cancer to an anti-neuroendocrine cancer therapy, the method comprising: (a) at a first time point: (i) determining the expression level of at least 36 biomarkers in a test sample from the subject, wherein the at least 38 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (iii) inputting each normalized expression level from step (a)(ii) into an algorithm to generate a first score; (b) at a second time point, wherein the second time point is after the first time point and after the administration of the anti-neuroendocrine therapy to the subject: (i) determining the expression level of the at least 36 biomarkers in a test sample from the subject; (ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and (iii) inputting each normalized expression level from step (b)(ii) into an algorithm to generate a second score; (c) comparing the first score and the second score; and (d) identifying that the subject is responsive to the anti-neuroendocrine cancer therapy when the second score is decreased as compared to the first score or identifying that the subject is not responsive to the anti-neuroendocrine cancer therapy when the second score is not decreased as compared to the first score.

### General Methods and Definitions

The following general methods and definitions can be applied to any of the preceding methods.

In some aspect, the test sample can comprise saliva.

Exemplary housekeeping genes include, but are not limited to, ATG4B, RHOA, TOX4, TPT1, and TXNIP. In some aspects, the housekeeping gene is RHOA.

Each of the biomarkers disclosed herein may have one or more transcript variants. The methods disclosed herein can measure the expression level of any one of the transcript variants for each biomarker.

In some aspects, determining the expression level of at least 36 biomarkers in a test sample from a subject can comprise contacting the test sample with a plurality of agents specific to detect the expression of the at least 36 biomarkers.

Accordingly, the present disclosure provides the use of a plurality of agents to detect the expression of at least 36 biomarkers in the manufacture of a kit for identifying the presence or absence of a neuroendocrine cancer by the methods described herein.

The present disclosure also provides the use of a plurality of agents to detect the expression of at least 36 biomarkers in the manufacture of a kit for identifying the risk of a subject having a neuroendocrine cancer by the methods described herein.

The present disclosure also provides the use of a plurality of agents to detect the expression of at least 36 biomarkers in the manufacture of a kit for determining whether a neuroendocrine cancer in a subject is stable or progressive by the methods described herein.

The present disclosure also provides the use of a plurality of agents to detect the expression of at least 36 biomarkers in the manufacture of a kit for determining the completeness of a surgery to remove a neuroendocrine cancer in a subject by the methods described herein.

The present disclosure also provides the use of a plurality of agents to detect the expression of at least 36 biomarkers in the manufacture of a kit for evaluating the response of a subject having a neuroendocrine cancer to an anti-neuroendocrine cancer therapy by the methods described herein.

The expression levels can be measured in a number of ways, including, but not limited to measuring the mRNA encoded by the selected genes; measuring the amount of protein encoded by the selected genes; measuring the activity of the protein encoded by the selected genes; or any combination thereof.

The biomarker can be RNA, cDNA, or protein. When the biomarker is RNA, the RNA can be reverse transcribed to produce cDNA (such as by RT-PCR), and the produced cDNA expression level is detected. The expression level of the biomarker can be detected by forming a complex between the biomarker and a labeled probe or primer. When the biomarker is RNA or cDNA, the RNA or cDNA is detected by forming a complex between the RNA or cDNA and a labeled nucleic acid probe or primer. The complex between the RNA or cDNA and the labeled nucleic acid probe or primer can be a hybridization complex.

As would be appreciated by the skilled artisan, gene expression can be detected by microarray analysis. Differential gene expression can also be identified or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In some aspects of the microarray technique, PCR amplified inserts of cDNA clones are applied to a substrate in a dense array. In some aspects, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In some aspects, the biomarkers (i.e. the NETsaliva transcripts and/or housekeeping genes) can be detected in a saliva sample using RNAseq. As would be appreciated by the skilled artisan, the first step in gene expression profiling by RNAseq is extracting RNA from a saliva sample followed by the reverse transcription of the RNA template into cDNA to generate the RNA libraries. Sequencing adapters are added. cDNA is then sequenced using a sequencing platform. Data is analyzed and expressed as transcripts per million.

In some aspects, the biomarkers (i.e. the NETsaliva transcripts and/or housekeeping genes) can be detected in a saliva sample using qRT-PCR. As would be appreciated by the skilled artisan, the first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

In some aspects wherein the biomarker is protein, the protein can be detected by forming a complex between the protein and a labeled antibody. The label can be any label for example a fluorescent label, chemiluminescence label, radioactive label, etc. Exemplary methods for protein detection include, but are not limited to, enzyme immunoassay (EIA), radioimmunoassay (RIA), Western blot analysis and enzyme linked immunoabsorbant assay (ELISA). For example, the biomarker can be detected in an ELISA, in which the biomarker antibody is bound to a solid phase and an enzyme-antibody conjugate is employed to detect and/or quantify biomarker present in a sample. Alternatively, a western blot assay can be used in which solubilized and separated biomarker is bound to nitrocellulose paper. The combination of a highly specific, stable liquid conjugate with a sensitive chromogenic substrate allows rapid and accurate identification of samples.

In some aspects, the methods described herein can have a specificity, sensitivity, and/or accuracy of at least 75%, 80%, 85%, 90%, 91%, 92%, 93%, 94%, 95%, 96%, 97%, 98%, or 99%.

In some aspects, the methods described herein can have a specificity (e.g. a specificity for identifying the presence or absence of a neuroendocrine cancer, a specificity for identifying whether a neuroendocrine cancer is stable or progressive, a specificity for identifying the completeness of surgery in a subject having a neuroendocrine cancer, or a specificity for evaluating the response of a subject having a neuroendocrine cancer to an anti-neuroendocrine cancer therapy) of at least 75%, 80%, 85%, 90%, 91%, 92%, 93%, 94%, 95%, 96%, 97%, 98%, or 99%.

In some aspects, the methods described herein can have a sensitivity (e.g. a sensitivity for identifying the presence or absence of a neuroendocrine cancer, a sensitivity for identifying whether a neuroendocrine cancer is stable or progressive, a sensitivity for identifying the completeness of surgery in a subject having a neuroendocrine cancer, or a sensitivity for evaluating the response of a subject having a neuroendocrine cancer to an anti-neuroendocrine cancer therapy) of at least 75%, 80%, 85%, 90%, 91%, 92%, 93%, 94%, 95%, 96%, 97%, 98%, or 99%.

In some aspects, the methods described herein can have a accuracy (e.g. an accuracy for identifying the presence or absence of a neuroendocrine cancer, an accuracy for identifying whether a neuroendocrine cancer is stable or progressive, an accuracy for identifying the completeness of surgery in a subject having a neuroendocrine cancer, or an accuracy for evaluating the response of a subject having a neuroendocrine cancer to an anti-neuroendocrine cancer therapy) of at least 75%, 80%, 85%, 90%, 91%, 92%, 93%, 94%, 95%, 96%, 97%, 98%, or 99%.

Any algorithm that can generate a score for a sample by assessing where that sample value falls onto a prediction model generated using different techniques, e.g., decision trees, can be used in the methods disclosed herein. The algorithm analyzes the data (i.e., expression levels) and then assigns a score. In some aspects, the algorithm can be a machine-learning algorithm. Exemplary algorithms that can be used in the methods disclosed herein can include, but are not limited to, XGB, Random Forest (RF), glmnet, cforest, CART, treebag, knn, nnet, SVM-radial, SVM-linear, NB, and mlp. In some aspects, the algorithm can be Random Forest In some aspects, the algorithm can be XGB (also called XGBoost). XGB is an implementation of gradient boosted decision trees designed for speed and performance. In some aspects, the algorithm can be Random Forest. In some aspects, the Random Forest algorithm can be a grid-search optimized Random-Forest algorithm. Random Forest is an implementation of an ensemble learning method for classification, regression and other tasks that operates by constructing a multitude of decision trees at training time.

In some aspects of the methods of the present disclosure, the machine learning algorithm can be trained using: a) the expression levels or normalized expression levels of the at least 36 biomarkers in at least one biological sample (e.g. saliva) from at least one subject who does not have a neuroendocrine cancer; and b) the expression levels or normalized expression levels of the at least 36 biomarkers in at least one biological sample (e.g. saliva) from at least one subject who has neuroendocrine cancer. That is, in some aspects, the machine learning algorithm is trained using the expression levels or normalized expression levels of the at least 36 biomarkers obtained from a plurality of reference samples obtained from subjects not having a neuroendocrine cancer and the expression levels or normalized expression levels of the at least 36 biomarkers from a plurality of reference samples obtained from subjects having neuroendocrine cancer.

In some aspects, one or more predetermined cutoff values can be derived from a plurality of reference samples obtained from subjects not having or not diagnosed with a neoplastic disease. The plurality of reference samples can be about 2 to about 500 samples, about 2 to about 200 samples, about 10 to about 100, or about 20 to about 80 samples.

In some aspects, determining a predetermined cutoff value can comprise inputting the normalized expression level of the NETsaliva transcripts from each reference sample into the same algorithm used in the methods described above, thereby generating a plurality of scores from the plurality of reference samples. The predetermined cutoff value can then be determined by taking the arithmetic mean of these scores. In some aspects, the reference samples can comprise saliva. In some aspects, the reference sample is of the same type as the test sample.

In some aspects of the methods of the present disclosure, a predetermined cutoff value can be calculated and/or selected using at least one receiver operating characteristic (ROC) curve. In some aspects of the methods of the present disclosure, a predetermined cutoff value can be calculated and/or selected to have any of the features described herein (e.g., a specific sensitivity, specificity, accuracy, or any combination thereof) using any method known in the art, as would be appreciated by the skilled artisan.

In some aspects, the methods described herein can further comprise treating a subject with an anti-neuroendocrine cancer therapy.

Accordingly, in some aspects, the methods described herein further comprise treating a subject identified as having a neuroendocrine cancer with an anti-neuroendocrine cancer therapy. In some aspects, the methods described herein further comprise treating a subject identified as having a progressive neuroendocrine cancer with at least one anti-neuroendocrine cancer therapy. In some aspects, the methods described herein further comprise treating a subject identified as having a high risk of a neuroendocrine cancer with at least one anti-neuroendocrine cancer therapy. In some aspects, the methods described herein further comprise treating a subject whose neuroendocrine cancer has not been completely removed by surgery with at least one anti-neuroendocrine cancer therapy.

In some aspects, the methods described further comprise treating a subject who is identified as not responding to an anti-neuroendocrine cancer therapy with a different anti-neuroendocrine cancer therapy. In some aspects, the methods described further comprise continuing to treat a subject who is identified as responding to an anti-neuroendocrine cancer therapy with the same anti-neuroendocrine cancer therapy.

In some aspects, anti-neuroendocrine cancer therapy can comprise active surveillance, surgery, cryotherapy, chemotherapy, targeted treatment, radiation therapy, or any combination thereof. Anti-neuroendocrine cancer therapy can comprise any therapeutic known in the art that is effective at treating neuroendocrine cancer.

As would be appreciated by the skilled artisan, active surveillance can include a doctor visit with a chromogranin A blood test and imaging scan about every 6 months. Active surveillance can also include imaging with ⁶⁸Ga-PET-SSA-CT scanning which may be done every 2^{nd} year.

As would be appreciated by the skilled artisan, surgery for neuroendocrine cancer patients can include a complete resection (R0 “curative” surgery).

As would be appreciated by the skilled artisan, cryotherapy (also called cryosurgery or cryoablation) is the use of very cold temperatures to freeze and kill neuroendocrine cancer cells, typically in the liver.

As would be appreciated by the skilled artisan, chemotherapy can comprise streptozotocin, doxorubicin, 5-FU, dacarbazine, temozolomide, capecitabine and oxaliplatin, or any combination thereof.

As would be appreciated by the skilled artisan, targeted therapy can comprise somatostatin analogues, everolimus, sunibinib and immunotherapy, or any combination thereof.

As would be appreciated by the skilled artisan, radiation therapy can comprise peptide receptor radionuclide therapy (PRRT).

As would be appreciated by the skilled artisan, if a neuroendocrine cancer has grown outside the primary tumor site, preventing or slowing the spread of the cancer to the liver or bones is a major goal of treatment. Liver and/or bone-directed treatment can include radiation therapy or the use of radiopharmaceuticals (e.g., Indium-111, Lutetium-177).

The sequence information of the neuroendocrine cancer biomarkers and housekeeping genes is shown in Table 1. Table 1 shows representative sequences for each of the neuroendocrine cancer biomarkers and housekeeping genes discussed herein. The skilled artisan would appreciate that in addition to the specific sequences shown in Table 1, other isoforms and variants of the variants can also be measured in the methods of the present disclosure to obtain an expression level of the biomarker or housekeeping gene.

TABLE 1
Neuroendocrine Cancer Biomarker/Housekeeper
Sequence Information

| Gene | RefSeq | SEQ ID |
|---|---|---|
| Name | Accession | NO: |
| AKAP8L | NM_014371.3 | 1 |
| APLP2 | NM_001142276.1 | 2 |
| ARAF1 | NM_001654.4 | 3 |
| BNIP3L | BNIP3L | 4 |
| | NM_004331.2 | |
| BRAF | NM_004333.4 | 5 |
| CD59 | NM_203331.2 | 6 |
| COMMD9 | NM_001101653.1 | 7 |
| CTGF | NM_001901.2 | 8 |
| FAM131A | NM_001171093.1 | 9 |
| FLJ10357 | NM_018071.4 | 10 |
| FZD7 | NM_003507.1 | 11 |
| GLT8D1 | NM_001010983.2 | 12 |
| KRAS | NM_004985.4 | 13 |
| LEO1 | NM_138792.3 | 14 |
| MORF4L2 | NM_001142418.1 | 15 |
| NUDT3 | NM_006703.3 | 16 |
| OAZ2 | NM_002537.3 | 17 |
| PANK2 | NM_024960.4 | 18 |
| PHF21A | NM_001101802.1 | 19 |
| PKD1 | NM_000296.3 | 20 |
| PLD3 | NM_001031696.3 | 21 |
| PNMA2 | NM_007257.5 | 22 |
| PQBP1 | NM_001032381.1 | 23 |
| RAF1 | NM_002880.3 | 24 |
| RNF41 | NM_001242826.1 | 25 |
| RSF1 | NM_016578.3 | 26 |
| RTN2 | NM_005619.4 | 27 |
| SMARCD3 | NM_001003801.1 | 28 |
| SSTR1 | NM_001049.2 | 29 |
| SSTR3 | NM_001051.4 | 30 |
| SST4 | NM_001052.2 | 31 |
| TECPR2 | NM_001172631.1 | 32 |
| TRMT112 | NM_016404.2 | 33 |
| VPS13C | NM_001018088.2 | 34 |
| WDFY3 | NM_014991.4 | 35 |
| ZXDC | NM_001040653.3 | 36 |
| ATG4B | NM_178326.3 | 37 |
| RHOA | NM_001313941.2 | 38 |
| TOX4 | NM_001303523.2 | 39 |
| TPT1 | NM_001286272.2 | 40 |
| TXNIP | NM_001313972.2 | 41 |

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In some aspects, a nucleic acid molecule or nucleic acid sequence comprises other kinds of nucleic acid structures including, but not limited to, a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees centigrade and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)); b. prognosis (predicting whether a patient will likely have a better or worse outcome at a pre-selected time in the future); c. therapy selection, d. therapeutic drug monitoring, and e. relapse monitoring. [00119]“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include bodily fluids such as saliva or lavage or any other specimen used for detection of disease.

The term “subject” as used herein refers to a mammal, preferably a human. In some aspects, the subject has at least one neuroendocrine cancer symptom. In some aspects, the subject has a predisposition or familial history for developing a neuroendocrine cancer. The subject could also be previously diagnosed with a neuroendocrine cancer and is tested for cancer recurrence.

“Treating” or treatment of a disease or condition refers to executing a protocol or treatment plan, which may include administering one or more therapeutic agents to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. In addition, “treating” or “treatment” does not require complete alleviation of signs or symptoms, does not require a cure, and specifically includes protocols or treatment plans that have only a marginal effect on the patient.

As used herein, “prevent”, “preventing” and the like describe stopping the onset of the disease, condition or disorder, or one or more symptoms or complications thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and 5-fold. The change may be statistically significant with a p value of 0.1, 0.05, 0.001, or 0.0001.

The term “stable disease” refers to a diagnosis for the presence of a neuroendocrine cancer, however the neuroendocrine cancer has been treated and remains in a stable condition, i.e., one that that is not progressive, as determined by imaging data and/or best clinical judgment.

The term “progressive disease” refers to a diagnosis for the presence of a highly active state of a neuroendocrine cancer, i.e., one has not been treated and is not stable or has been treated and has not responded to therapy, or has been treated and active disease remains, as determined by imaging data and/or best clinical judgment.

The term “neoplastic disease” refers to any abnormal growth of cells or tissues being either benign (non-cancerous) or malignant (cancerous). For example, the neoplastic disease can be a neuroendocrine cancer.

The term “neoplastic tissue” refers to a mass of cells that grow abnormally.

The term “non-neoplastic tissue” refers to a mass of cells that grow normally.

As used herein, the term “about” when used in conjunction with numerical values and/or ranges generally refers to those numerical values and/or ranges near to a recited numerical value and/or range. In some instances, the term “about” can mean within +10% of the recited value. For example, in some instances, “about 100 [units]” can mean within +10% of 100 (e.g., from 90 to 110).

### Examples

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Derivation of a 36-Marker Gene Panel

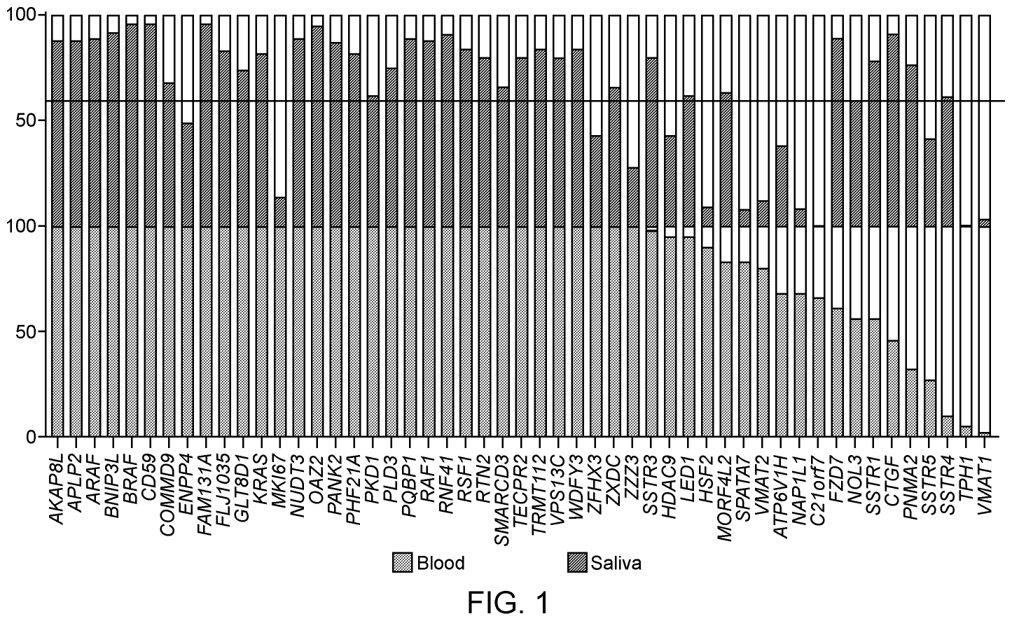

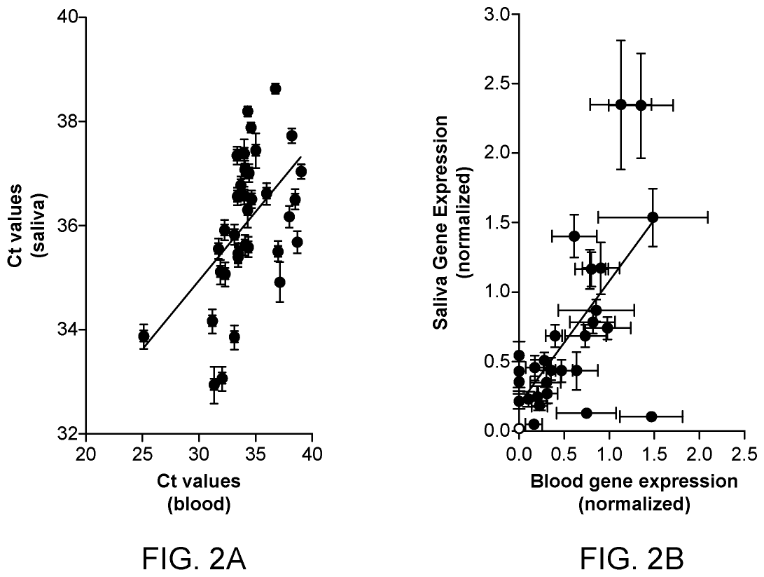

The panel of NETsaliva transcripts was derived from evaluating gene expression in matched blood and saliva samples collected from 44 neuroendocrine cancer patients, including the expression of biomarkers previously identified in blood samples from neuroendocrine cancer patients (see US 2014-0066328A1, US 2016-0076106A1 and US 2019-0160189 A1). Forty-nine (96%) of the previously identified genes were detectable, but only 36 of these were detectable in >60% of saliva samples (FIG. 1). These 36 genes were highly correlated both in terms of measurement (Ct values) as well as when expressed as normalized values. The correlation between blood and saliva Ct values was r=0.52. (p=0.0011, FIG. 2A) and for normalized values the Pearson r value was 0.51 (p=0.0014; FIG. 2B).

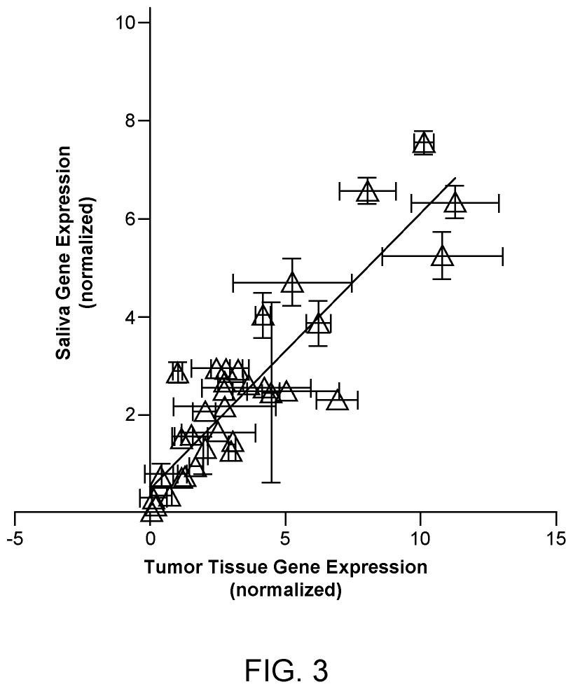

These genes were demonstrated to be highly expressed in neuroendocrine cancer tumor tissue and there was a significant correlation (r=0.89, p<0.0001) with saliva gene expression identifying that saliva could be used to effectively function as a liquid biopsy (FIG. 3).

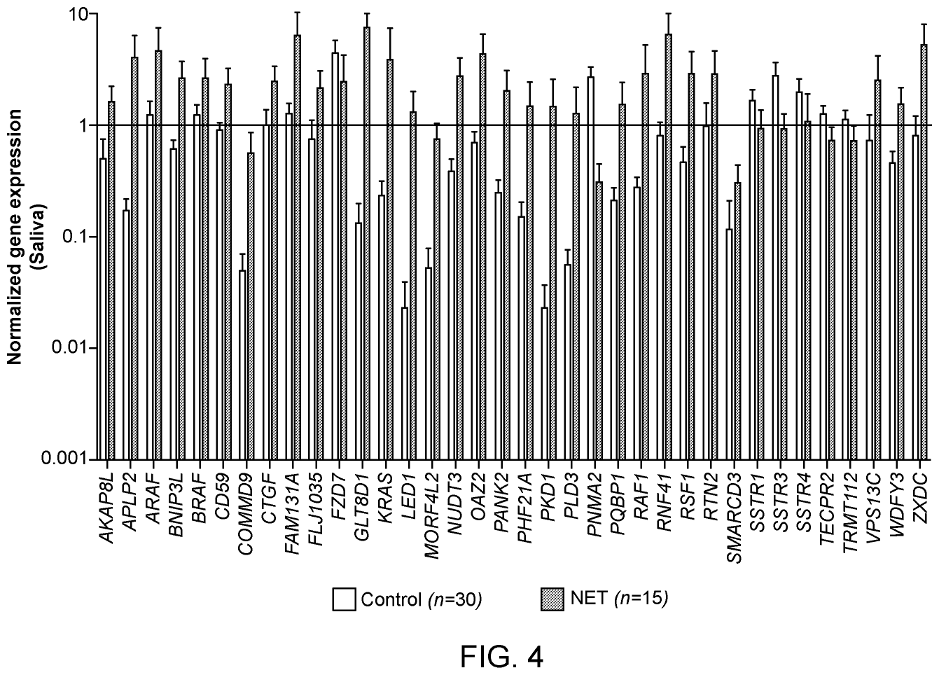

Evaluation of transcripts in a preliminary dataset of saliva samples from age (average 72 years) and sex (8M:7F) matched neuroendocrine cancers (n=15) and normal saliva (n=30) confirmed expression of the 36 genes as markers of neuroendocrine cancer (FIG. 4). These data demonstrate the candidate target transcripts are produced by neoplastic transformed neuroendocrine cells and are detectable in saliva.

An artificial intelligence model of neuroendocrine cancer disease was built using normalized gene expression of these 36 markers (Table 2) in saliva from Controls (n=274), and neuroendocrine cancer (n=76) samples. The dataset was randomly split into training and testing partitions for model creation and validation respectively. Twelve algorithms were evaluated (XGB, Random Forest (RF), glmnet, cforest, CART, treebag, knn, nnet, SVM-radial, SVM-linear, NB and mlp). The top performing algorithm (RF—“random forest”) best predicted the training data. In the test set, RF produced probability scores that predicted the sample. Each probability score reflects the “certainty” of an algorithm that an unknown sample belongs to either “Control” or “Neuroendocrine Cancer” class. For example, an unknown sample S1 can have the following probability vector [Control=20%, NET=80%]. This sample would be considered a neuroendocrine cancer sample.

TABLE 2
36 Neuroendocrine cancer saliva marker gene panel (not including the housekeeping gene)

| Neuroendocrine cancer | | NCBI | | | | |
|---|---|---|---|---|---|---|
| gene marker | | Chromosome | UniGene | | Length | Exon |
| Symbol | Name | location | ID | RefSeq | (bp) | Boundary |
| AKAP8L | A kinase (PRKA) | Chr. 19: | Hs.399800 | NM_014371 | 75 | 12-13 |
| | anchor protein 8- | 15490859- | | | | |
| | like | 15529833 | | | | |
| | | [19p13.12] | | | | |
| APLP2 | amyloid beta (A4) | Chr. 11 - | Hs.370247 | NM_001142276.1 | 102 | 14-15 |
| | precursor-like | 129939716- | | | | |
| | protein 2 | 130014706 | | | | |
| | | [11q24.3] | | | | |
| ARAF1 | v-raf murine | Chr. X - | Hs.446641 | NM_001654.3 | 74 | 10-11 |
| | sarcoma 3611 viral | 47420578- | | | | |
| | oncogene homolog | 47431320 | | | | |
| | | [Xp11.3] | | | | |
| BNIP3L | BCL2/adenovirus | Chr. 8: | Hs.131226 | NM_004331 | 69 | 2-3 |
| | E1B 19 kDa | 26240523- | | | | |
| | interacting protein | 26270644 | | | | |
| | 3-like | [8p21.2] | | | | |
| BRAF | v-raf murine | Chr. 7 - | Hs.550061 | NM_004333.4 | 77 | 1-2 |
| | sarcoma viral | 140433812- | | | | |
| | oncogene homolog | 140624564 | | | | |
| | B1 | [7q34] | | | | |
| CD59 | CD59 molecule, | Chr. 11 - | Hs.278573 | NM_203331.2 | 70 | 3-4 |
| | complement | 33724556- | | | | |
| | regulatory protein | 33758025 | | | | |
| | | [11p13] | | | | |
| COMMD9 | COMM domain | Chr. 11: | Hs.279836 | NM_001101653 | 85 | 2-3 |
| | containing 9 | 36293842- | | | | |
| | | 36310999 | | | | |
| | | [11p13] | | | | |
| CTGF | connective tissue | Chr. 6 - | Hs.410037 | NM_001901.2 | 60 | 4-5 |
| | growth factor | 132269316- | | | | |
| | | 132272518 | | | | |
| | | [6q23.2] | | | | |
| FAM131A | family with | Chr. 3: | Hs.591307 | NM_001171093 | 64 | 4-5 |
| | sequence similarity | 184053717- | | | | |
| | 131, member A, | 184064063 | | | | |
| | transcript variant 2 | [3q27.1] | | | | |
| FLJ10357 | Rho guanine | Chr. 14: | Hs.35125 | NM_018071 | 102 | 16-17 |
| (ARHGEF40) | nucleotide | 21538527- | | | | |
| | exchange factor (GEF) | 21558036 | | | | |
| | 40 (ARHGEF40) | [14q11.2] | | | | |
| FZD7 | frizzled homolog 7 | Chr. 2 - | Hs.173859 | NM_003507.1 | 70 | 1-1 |
| | (Drosophila) | 202899310- | | | | |
| | | 202903160 | | | | |
| | | [2q33.1] | | | | |
| GLT8D1 | glycosyltransferase | Chr. 3: | Hs.297304 | NM_001010983 | 87 | 4-5 |
| | 8 domain | 52728504- | | | | |
| | containing 1, | 52740048 | | | | |
| | transcript variant 3 | [3p21.1] | | | | |
| KRAS | v-Ki-ras2 Kirsten rat | Chr. 12 - | Hs.505033 | NM_004985.3 | 130 | 4-5 |
| | sarcoma viral | 25358180- | | | | |
| | oncogene homolog | 25403854 | | | | |
| | | [12p12.1] | | | | |
| LEO1 | Leo1, Paf1/RNA | Chr. 15: | Hs.567662 | NM_138792 | 122 | 10-11 |
| | polymerase II | 52230222- | | | | |
| | complex component | 52263958 | | | | |
| | homolog (S. cerevisiae) | [15q21.2] | | | | |
| MORF4L2 | mortality factor 4 | Chr. X: | Hs.326387 | NM_001142418 | 101 | 2-3 |
| | like 2, transcript | 102930426- | | | | |
| | variant 1 | 102943086 | | | | |
| | | [Xq22.2] | | | | |
| NUDT3 | nudix (nucleoside | Chr. 6: | Hs.188882 | NM_006703 | 62 | 2-3 |
| | diphosphate linked | 34255997- | | | | |
| | moiety X)-type | 34360441 | | | | |
| | motif 3 | [6p21.31] | | | | |
| OAZ2 | ornithine | Chr. 15: | Hs.713816 | NM_002537 | 96 | 1-2 |
| | decarboxylase | 64979773- | | | | |
| | antizyme 2 | 64995462 | | | | |
| | | [15q22.31] | | | | |
| PANK2 | pantothenate | Chr. 20: | Hs.516859 | NM_024960 | 126 | 4-5 |
| | kinase 2 | 3869486- | | | | |
| | | 3904502 | | | | |
| | | [20p13] | | | | |
| PHF21A | PHD finger protein | Chr. 11: | Hs.502458 | NM_001101802 | 127 | 16-17 |
| | 21A, transcript | 45950870- | | | | |
| | variant 1 | 46142985 | | | | |
| | | [11p11.2] | | | | |
| PKD1 | polycystic kidney | Chr. 16: | Hs.75813 | NM_000296 | 110 | 16-17 |
| | disease 1 (autosomal | 2138711- | | | | |
| | dominant), transcript | 2185899 | | | | |
| | variant 2 | [16p13.3] | | | | |
| PLD3 | phospholipase D | Chr. 19: | Hs.257008 | NM_001031696 | 104 | 6-7 |
| | family, member 3, | 40854332- | | | | |
| | transcript variant 1 | 40884390 | | | | |
| | | [19q13.2] | | | | |
| PNMA2 | paraneoplastic | Chr. 8 - | Hs.591838 | NM_007257.5 | 93 | 1-2 |
| | antigen MA2 | 26362196- | | | | |
| | | 26371483 | | | | |
| | | [8p21.2] | | | | |
| PQBP1 | polyglutamine | Chr. X: | Hs.534384 | NM_001032381 | 68 | 2-3 |
| | binding protein 1, | 48755195- | | | | |
| | transcript variant 2 | 48760422 | | | | |
| | | [Xp11.23] | | | | |
| RAF1 | v-raf-1 murine | Chr. 3 - | Hs.159130 | NM_002880.3 | 90 | 7-8 |
| | leukemia viral | 12625100- | | | | |
| | oncogene homolog 1 | 12705700 | | | | |
| | | [3p25.2] | | | | |
| RNF41 | ring finger protein | Chr. 12: | Hs.524502 | NM_001242826 | 72 | 2-3 |
| | 41, transcript | 56598285- | | | | |
| | variant 4 | 56615735 | | | | |
| | | [12q13.3] | | | | |
| RSF1 | remodeling and | Chr. 11: | Hs.420229 | NM_016578 | 60 | 7-8 |
| | spacing factor 1 | 77377274- | | | | |
| | | 77531880 | | | | |
| | | [11q14.1] | | | | |
| RTN2 | reticulon 2, | Chr. 19: | Hs.47517 | NM_005619 | 87 | 9-10 |
| | transcript variant 1 | 45988550- | | | | |
| | | 46000313 | | | | |
| | | [19q13.32] | | | | |
| SMARCD3 | SWI/SNF related, | Chr. 7: | Hs.647067 | NM_001003801 | 109 | 8-9 |
| | matrix associated, | 150936059- | | | | |
| | actin dependent | 150974231 | | | | |
| | regulator of | [7q36.1] | | | | |
| | chromatin, subfamily | | | | | |
| | d, member 3, | | | | | |
| | transcript variant 3 | | | | | |
| SSTR1 | somatostatin | Chr. 14: | Hs.248160 | NM_001049 | 85 | 3-3 |
| | receptor 1 | 38677204- | | | | |
| | | 38682268 | | | | |
| | | [14q21.1] | | | | |
| SSTR3 | somatostatin | Chr. 22: | Hs.225995 | NM_001051 | 84 | 2-2 |
| | receptor 3 | 37602245- | | | | |
| | | 37608353 | | | | |
| | | [22q13.1] | | | | |
| SSTR4 | somatostatin | Chr. 20: | Hs.673846 | NM_001052 | 104 | 1-1 |
| | receptor 4 | 23016057- | | | | |
| | | 23017314 | | | | |
| | | [20p11.21] | | | | |
| TECPR2 | tectonin beta- | Chr. 14: | Hs.195667 | NM_001172631 | 61 | 12-13 |
| | propeller repeat | 102829300- | | | | |
| | containing 2, | 102968818 | | | | |
| | transcript variant 2 | [14q32.31] | | | | |
| TRMT112 | tRNA | Chr. 11: | Hs.333579 | NM_016404 | 91 | 1-2 |
| | methyltransferase | 64084163- | | | | |
| | 11-2 homolog (S. | 64085033 | | | | |
| | cerevisiae) | [11q13.1] | | | | |
| VPS13C | vacuolar protein | Chr. 15: | Hs.511668 | NM_001018088 | 65 | 69-70 |
| | sorting 13 homolog | 62144588- | | | | |
| | C (S. cerevisiae), | 62352647 | | | | |
| | transcript variant 2B | [15q22.2] | | | | |
| WDFY3 | WD repeat and | Chr. 4: | Hs.480116 | NM_014991 | 81 | 64-65 |
| | FYVE domain | 85590690- | | | | |
| | containing 3 | 85887544 | | | | |
| | | [4q21.23] | | | | |
| ZXDC | zinc finger C, | Chr. 3: | Hs.440049 | NM_001040653 | 61 | 1-2 |
| | transcript variant 2 | 126156444- | | | | |
| | | 126194762 | | | | |
| | | [3q21.3] | | | | |

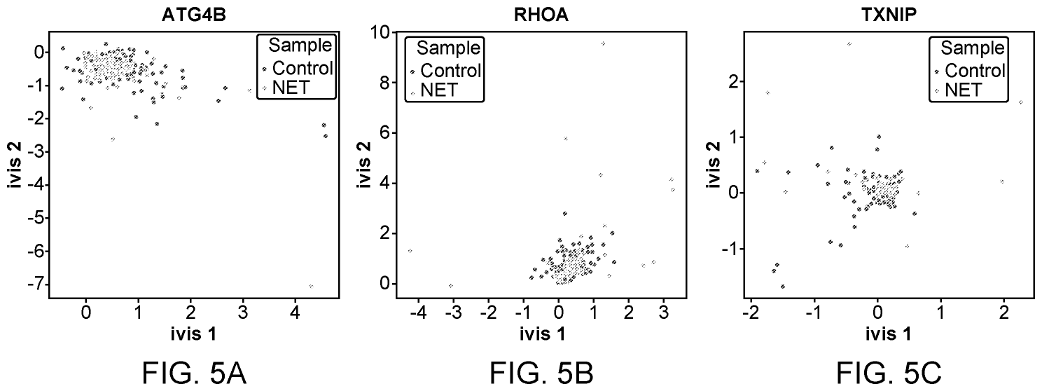

The 36 marker genes identified by the Random Forest Machine algorithm were visualized in the derivation cohort of n=274 control samples and 76 cancer samples using the IVIS algorithm (FIG. 5A-C).

### Example 2. Clinical Utility

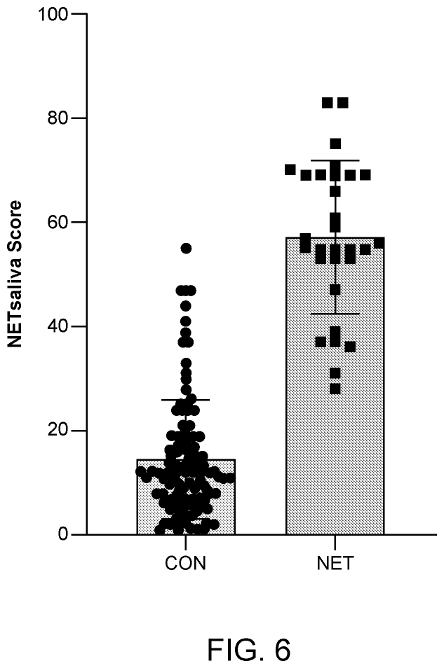

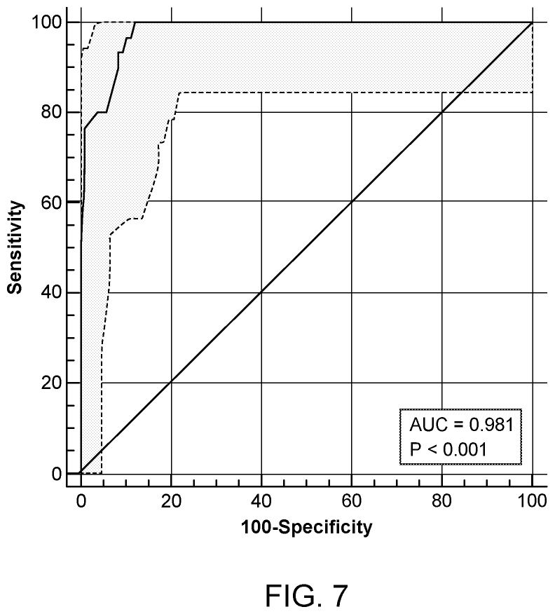

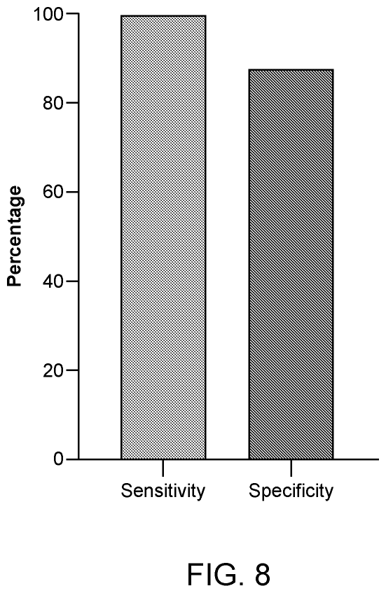

The NETsaliva scores were significantly (p<0.001) elevated in neuroendocrine cancers (57±14%) compared to controls (15±11%) (FIG. 6). The data (receiver operator curve analysis and metrics) for the utility of the test to differentiate patients with neuroendocrine cancer (n=30) from controls (n=108) in the validation is included in FIG. 7. The score exhibited an area under the curve (AUROC) of 0.98. The metrics are: sensitivity: 100% and specificity: 88% (FIG. 8). The Youden index J is 0.88 and the Z-statistic for differentiating controls was 54.9.

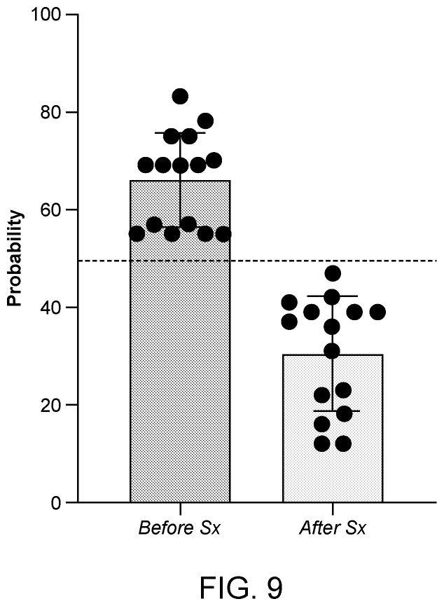

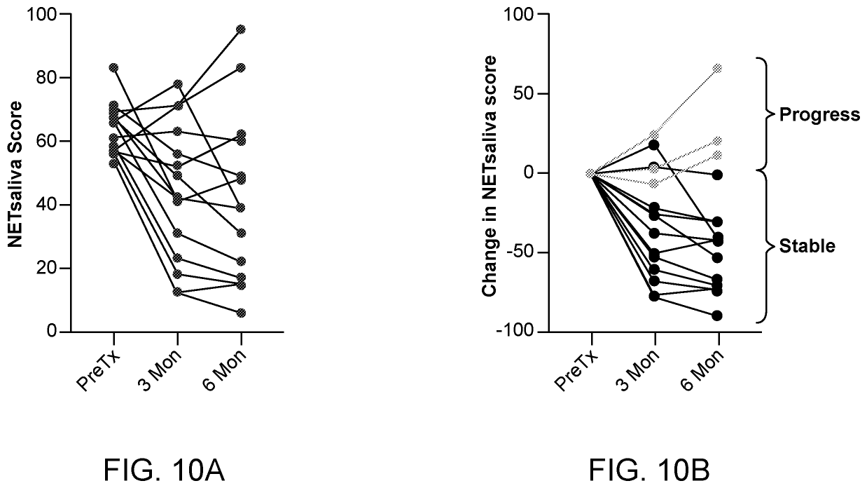

Specific evaluation of a neuroendocrine carcinoma cohort before and after surgery identified that complete removal of a tumor and no evidence of disease was associated with a significant decrease (p<0.0001) in the NETsaliva score (FIG. 9). Levels were not significantly different to controls. Evaluation of separate cohort identified that patients who underwent and responded to therapy exhibited a significant lower score (p<0.001) that those diagnosed with disease (FIG. 10). Therapies included targeted therapy and PRRT. The tool can therefore accurately identify treatment response in neuroendocrine cancer disease.

### EQUIVALENTS

While the present inventions have been described in conjunction with the specific embodiments set forth above, many alternatives, modifications and other variations thereof will be apparent to those of ordinary skill in the art. All such alternatives, modifications and variations are intended to fall within the spirit and scope of the present inventions.

What is claimed is:

1. A method of identifying the presence or absence of a neuroendocrine cancer in a subject in need thereof, the method comprising:
(a) determining the expression level of at least 36 biomarkers from a saliva sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene;
(b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC;
(c) inputting each normalized expression level from step (b) into an algorithm to generate a score;
(d) comparing the score to a predetermined cutoff value; and
(e) identifying the presence of the neuroendocrine cancer in the subject when the score is greater than or equal to the predetermined cutoff value or determining the absence of the neuroendocrine cancer in the subject when the score is less than the predetermined cutoff value.

2. The method of claim 1, wherein the predetermined cutoff value is 26% on a scale of 0-100%.

3. A method of determining whether a neuroendocrine cancer in a subject is stable or progressive, the method comprising:
(a) determining the expression level of at least 36 biomarkers from a saliva sample from the subject, wherein the at least 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene;
(b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC;
(c) inputting each normalized expression level from step (b) into an algorithm to generate a score;
(d) comparing the score to a predetermined cutoff value; and
(e) determining that the neuroendocrine cancer is progressive when the score is greater than or equal to the predetermined cutoff value or determining that the neuroendocrine cancer is stable when the score is less than the predetermined cutoff value.

4. The method of claim 3, wherein the predetermined cutoff value is 50% on a scale of 0 to 100%.

5. A method of determining the completeness of surgery to remove a neuroendocrine cancer in a subject, the method comprising:
(a) determining the expression level of at least 36 biomarkers from a saliva sample from the subject after the surgery, wherein the 36 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene;
(b) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC;
(c) inputting each normalized expression level from step (b) into an algorithm to generate a score;
(d) comparing the score to a predetermined cutoff value; and
(e) identifying that the neuroendocrine cancer is not completely removed when the score is greater than or equal to the predetermined cutoff value or identifying that the neuroendocrine cancer is completely removed based when the score is less than the predetermined cutoff value.

6. The method of claim 5, wherein the predetermined cutoff value is 50% on a scale of 0 to 100%.

7. A method of evaluating the response of a subject having a neuroendocrine cancer to an anti-neuroendocrine cancer therapy, the method comprising:
(a) at a first time point:
(i) determining the expression level of at least 36 biomarkers from a saliva sample from the subject, wherein the at least 38 biomarkers comprise AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, ZXDC, and a housekeeping gene;
(ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and
(iii) inputting each normalized expression level from step (a)(ii) into an algorithm to generate a first score;
(b) at a second time point, wherein the second time point is after the first time point and after the administration of the anti-neuroendocrine therapy to the subject:
(i) determining the expression level of the at least 36 biomarkers from a saliva sample from the subject;
(ii) normalizing the expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC to the expression level of the housekeeping gene, thereby obtaining a normalized expression level of each of AKAP8L, APLP2, ARAF, BNIP3L, BRAF, CD59, COMMD9, CTGF, FAM131A, FLJ10357/ARHGEF40, FZD7, GLT8D1, KRAS, LEO1, MORF4L2, NUDT3, OAZ2, PANK2, PHF21A, PKD1, PLD3, PNMA2, PQBP1, RAF1, RNF41, RSF1, RTN2, SMARCD3, SSTR1, SSTR3, SSTR4, TECPR2, TRMT112, VPS13C, WDFY3, and ZXDC; and
(iii) inputting each normalized expression level from step (b)(ii) into an algorithm to generate a second score;
(c) comparing the first score and the second score; and
(d) identifying that the subject is responsive to the anti-neuroendocrine cancer therapy when the second score is decreased as compared to the first score or identifying that the subject is not responsive to the anti-neuroendocrine cancer therapy when the second score is not decreased as compared to the first score.

8. The method of claim 7, wherein the subject is identified as responsive to the anti-neuroendocrine cancer therapy when the second is at least 5% less than the first score.

9. The method of any one of the preceding claims, wherein the housekeeping gene is selected from the group consisting of ATG4B, RHOA, TOX4, TPT1, and TXNIP.

10. The method of claim 2, wherein the housekeeping gene is RHOA.

11. The method of any one of the preceding claims, having a sensitivity of at least 90%.

12. The method of any one of the preceding claims, having a specificity of at least 90%.

13. The method of any one of the preceding claims, wherein at least one of the at least 36 biomarkers is RNA, cDNA, or protein.

14. The method of claim 13, wherein when the biomarker is RNA, the RNA is reverse transcribed to produce cDNA, and the produced cDNA expression level is detected.

15. The method of any one of the preceding claims, wherein the expression level of the biomarker is detected by forming a complex between the biomarker and a labeled probe or primer.

16. The method of claim 13, wherein when the biomarker is protein, the protein detected by forming a complex between the protein and a labeled antibody.

17. The method of claim 16, wherein the label is a fluorescent label.

18. The method of claim 13, wherein when the biomarker is RNA or cDNA, the RNA or cDNA is detected by forming a complex between the RNA or cDNA and a labeled nucleic acid probe or primer.

19. The method of claim 18, wherein the label is a fluorescent label.

20. The method of claim 18 or claim 19, wherein the complex between the RNA or cDNA and the labeled nucleic acid probe or primer is a hybridization complex.

21. The method of any one of the preceding claims, wherein the first predetermined cutoff value is derived from a plurality of reference samples obtained from subjects not having or not diagnosed with a neoplastic disease.

22. The method of claim 21, wherein the neoplastic disease is neuroendocrine cancer.

23. The method of any one of the preceding claims, wherein the algorithm is XGB, RF, glmnet, cforest, CART, treebag, knn, nnet, SVM-radial, SVM-linear, NB, or mlp.

24. The method of claim 22, wherein the algorithm is RF, preferably wherein the RF algorithm is a grid-search optimized Random-Forest.

25. The method of claim 24, wherein the machine learning algorithm is trained using the expression levels or normalized expression levels of the at least 36 biomarkers obtained from a plurality of reference samples obtained from subjects not having a neuroendocrine cancer and the expression levels or normalized expression levels of the at least 36 biomarkers from a plurality of reference samples obtained from subjects having neuroendocrine cancer.

26. The method of any one of the preceding claims, further comprising treating the subject identified as having a neuroendocrine cancer with at least one anti-neuroendocrine cancer therapy.

27. The method of any one of the preceding claims, wherein the anti-neuroendocrine cancer therapy comprises active surveillance, surgery, cryotherapy, chemotherapy, targeted treatment, radiation therapy, or any combination thereof.

28. The method of claim 27, wherein the targeted therapy comprises somatostatin analogue therapy, everolimus, sunitinib, immunotherapy or any combination thereof.

29. The method of claim 27, wherein the chemotherapy comprises capecitabine, temozolomide, or any combination thereof.

30. The method of claim 27, wherein the radiation treatment comprises peptide receptor radionuclide therapy (PRRT).

31. The method of any one of the preceding claims, wherein the first time point is prior to the administration of the therapy to the subject.

32. The method of any one of the preceding claims, wherein the first time point is after the administration of the therapy to the subject.

33. The method of any one of the preceding claims, wherein the saliva sample is self-collected saliva into a container with stabilization fluid.


SEQUENCE LISTING (41 sequences)

SEQ ID NO: 1 - RNA, 2231 nt, Homo sapiens
  source 1..2231: mol_type = mRNA
actgatatgaggaggcatagagatagacagcggttccttccaatagacgtgaagccgaggccggtatgagccaatgcggtcgggaggcggggctcgggtgtgtgtggaggggaccctgtggttagcagcagctatcgcagcgtcggatgttcagagcagcagaagccggcgtcgtcggatgttgtgttgcccgccaccatgagctacacaggctttgtccagggatctgaaaccactttgcagtcgacatactcggataccagcgctcagcccacctgtgattatggatatggaacttggaactctgggacaaatagaggctacgagggctatggctatggctatggctatggccaggataacaccaccaactatgggtatggtatggccacttcacactcttgggaaatgcctagctctgacacaaatgcaaacactagtgcctcgggtagcgccagtgccgattccgttttatccagaattaaccagcgcttagatatggtgccgcatttggagacagacatgatgcaaggaggcgtgtacggctcaggtggagaaaggtatgactcttatgagtcctgcgactcgagggccgtcctgagtgagcgcgacctgtaccggtcaggctatgactacagcgagcttgaccctgagatggaaatggcctatgagggccaatacgatgcctaccgcgaccagttccgcatgcgtggcaacgacaccttcggtcccagggcacagggctgggcccgggatgcccggagcggccggccaatggcctcaggctatgggcgcatgtgggaagaccccatgggggcccggggccagtgcatgtctggtgcctctcggctgccctccctcttctcccagaacatcatccccgagtacggcatgttccagggcatgcgaggtgggggcgccttcccgggcggctcccgctttggtttcgggtttggcaatggcatgaagcagatgaggcggacctggaagacctggaccacagccgacttccgaaccaagaagaagaagagaaagcagggcggcagtcctgatgagccagatagcaaagccacccgcacggactgctcggacaacagcgactcagacaatgatgagggcaccgagggggaagccacagagggccttgaaggcaccgaggctgtggagaagggctccagagtggacggagaggatgaggagggaaaagaggatgggagagaagaaggcaaagaggatccagagaagggggccctaaccacccaggatgaaaatggccagaccaagcgcaagttgcaggcaggcaagaagagtcaggacaagcagaaaaagcggcagcgagaccgcatggtggaaaggatccagtttgtgtgttctctgtgcaaataccggaccttctatgaggacgagatggccagccatcttgacagcaagttccacaaggaacactttaagtacgtaggcaccaagctccctaagcagacggctgactttctgcaggagtacgtcactaacaagaccaagaagacagaggagctccgaaaaaccgtggaggaccttgatggcctcatccagcaaatctacagagaccaggatctgacccaggaaattgccatggagcattttgtgaagaaggtggaggcagcccattgtgcagcctgcgacctcttcattcccatgcagtttgggatcatccagaagcatctgaagaccatggatcacaaccggaaccgcaggctcatgatggagcagtccaagaagtcctccctcatggtggcccgcagtattctcaacaacaagctcatcagcaagaagctggagcgctacctgaagggcgagaaccctttcaccgacagccccgaggaggagaaggagcaggaggaggctgagggcggtgccctggacgagggggcgcagggcgaagcggcagggatctcggagggcgcagagggcgtgccggcgcagcctcccgtgcccccagagccagcccccggggccgtgtcgccgccaccgccgccgcccccagaggaggaggaggagggcgccgtgcccttgctgggaggggcgctgcaacgccagatccgcggcatcccgggcctcgacgtggaggacgacgaggagggcggcgggggcgccccgtgacccgagctcggggcgggcggagcccgcgtggccgaagctggaaaccaaacctaataaagttttcccatcccaccaaaaaaaaaaaaaaaaaa

SEQ ID NO: 2 - RNA, 3791 nt, Homo sapiens
  source 1..3791: mol_type = mRNA
agaaggagggcgtggtaatatgaagtcagttccggttggtgtaaaacccccggggcggcggcgaactggctttagatgcttctgggtcgcggtgtgctaagcgaggagtccgagtgtgtgagcttgagagccgcgcgctagagcgacccggcgagggatggcggccaccgggaccgcggccgccgcagccacgggcaggctcctgcttctgctgctggtggggctcacggcgcctgccttggcgctggccggctacatcgaggctcttgcagccaatgccggaacaggatttgctgttgctgagcctcaaatcgcaatgttttgtgggaagttaaatatgcatgtgaacattcagactgggaaatgggaacctgatccaacaggcaccaagagctgctttgaaacaaaagaagaagttcttcagtactgtcaggagatgtatccagagctacagatcacaaatgtgatggaggcaaaccagcgggttagtattgacaactggtgccggagggacaaaaagcaatgcaagagtcgctttgttacacctttcaagtgtctcgtgggtgaatttgtaagtgatgtcctgctagttccagaaaagtgccagtttttccacaaagagcggatggaggtgtgtgagaatcaccagcactggcacacggtagtcaaagaggcatgtctgactcagggaatgaccttatatagctacggcatgctgctcccatgtggggtagaccagttccatggcactgaatatgtgtgctgccctcagacaaagattattggatctgtgtcaaaagaagaggaagaggaagatgaagaggaagaggaagaggaagatgaagaggaagactatgatgtttataaaagtgaatttcctactgaagcagatctggaagacttcacagaagcagctgtggatgaggatgatgaggatgaggaagaaggggaggaagtggtggaggaccgagattactactatgacaccttcaaaggagatgactacaatgaggagaatcctactgaacccggcagcgacggcaccatgtcagacaaggaaattactcatgatgtcaaagctgtctgctcccaggaggcgatgacggggccctgccgggccgtgatgcctcgttggtacttcgacctctccaagggaaagtgcgtgcgctttatatatggtggctgcggcggcaacaggaacaattttgagtctgaggattattgtatggctgtgtgtaaagcgatgattcctccaactcctctgccaaccaatgatgttgatgtgtatttcgagacctctgcagatgataatgagcatgctcgcttccagaaggctaaggagcagctggagattcggcaccgcaaccgaatggacagggtaaagaaggaatgggaagaggcagagcttcaagctaagaacctccccaaagcagagaggcagactctgattcagcacttccaagccatggttaaagctttagagaaggaagcagccagtgagaagcagcagctggtggagacccacctggcccgagtggaagctatgctgaatgaccgccgtcggatggctctggagaactacctggctgccttgcagtctgacccgccacggcctcatcgcattctccaggccttacggcgttatgtccgtgctgagaacaaagatcgcttacataccatccgtcattaccagcatgtgttggctgttgacccagaaaaggcggcccagatgaaatcccaggtgatgacacatctccacgtgattgaagaaaggaggaaccaaagcctctctctgctctacaaagtaccttatgtagcccaagaaattcaagaggaaattgatgagctccttcaggagcagcgtgcagatatggaccagttcactgcctcaatctcagagacccctgtggacgtccgggtgagctctgaggagagtgaggagatcccaccgttccaccccttccaccccttcccagccctacctgagaacgaaggatctggagtgggagagcaggatgggggactgatcggtgccgaagagaaagtgattaacagtaagaataaagtggatgaaaacatggtcattgacgagactctggatgttaaggaaatgattttcaatgccgagagagttggaggcctcgaggaagagcgggaatccgtgggcccactgcgggaggacttcagtctgagtagcagtgctctcattggcctgctggtcatcgcagtggccattgccacggtcatcgtcatcagcctggtgatgctgaggaagaggcagtatggcaccatcagccacgggatcgtggaggttgatccaatgctcaccccagaagagcgtcacctgaacaagatgcagaaccatggctatgagaaccccacctacaaatacctggagcagatgcagatttaggtggcagggagcgcggcagccctggcggagggatgcaggtgggccggaagatcccacgattccgatcgactgccaagcagcagccgctgccaggggctgcgtctgacatcctgacctcctggactgtaggactatataaagtactactgtagaactgcaatttccattcttttaaatgggtgaaaaatggtaatataacaatatatgatatataaaccttaaatgaaaaaaatgatctattgcagatatttgatgtagttttcttttttaaattaatcagaaaccccacttccattgtattgtctgacacatgctctcaatatataataaatgggaaatgtcgattttcaataatagacttatatgcaggctgtcgttccggttatgttgtgtaagtcaactcttcagcctcattcactgtcctggcttttatttaaagaaaaaaaaggcagtattccctttttaaatgagctttcaggaagttgctgagaaatggggtggaatagggaactgtaatggccactgaagcacgtgagagaccctcgcaaaatgatgtgaaaggaccagtttcttgaagtccagtgtttccacggctggatacctgtgtgtctccataaaagtcctgtcaccaaggacgttaaaggcattttattccagcgtcttctagagagcttagtgtatacagatgagggtgtccgctgctgctttccttcggaatccagtgcttccacagagattagcctgtagcttatatttgacattcttcactgtctgttgtttacctaccgtagctttttaccgttcacttccccttccaactatgtccagatgtgcaggctcctcctctctggactttctccaaaggcactgaccctcggcctctactttgtcccctcacctccaccccctcctgtcaccggccttgtgacattcactcagagaagaccacaccaaggaggcggccgctggcccaggagagaacacggggaggtttgtttgtgtgaaaggaaagtagtccaggctgtccctgaaactgagtctgtggacactgtggaaagctttgaacaattgtgttttcgtcacaggagtctttgtaatgcttgtacagttgatgtcgatgctcactgcttctgctttttctttctttttattttaaatctgaaggttctggtaacctgtggtgtatttttattttcctgtgactgtttttgttttgtttttttcctttttcctcccctttgaccctattcatgtctctacccactatgcacagattaaacttcacctacaaactccttaatatgatctgtggagaatgtacacagtttaaacacatcaataaatactttaacttccaccgagaaaaaaaaaaaaaaaa

SEQ ID NO: 3 - RNA, 2522 nt, Homo sapiens
  source 1..2522: mol_type = mRNA
cttgacagacgtgaccctgacccaataagggtggaaggctgagtcccgcagagccaataacgagagtccgagaggcgacggaggcggactctgtgaggaaacaagaagagaggcccaagatggagacggcggcggctgtagcggcgtgacaggagccccatggcacctgcccagccccacctcagcccatcttgacaaaatctaaggctccatggagccaccacggggcccccctgccaatggggccgagccatcccgggcagtgggcaccgtcaaagtatacctgcccaacaagcaacgcacggtggtgactgtccgggatggcatgagtgtctacgactctctagacaaggccctgaaggtgcggggtctaaatcaggactgctgtgtggtctaccgactcatcaagggacgaaagacggtcactgcctgggacacagccattgctcccctggatggcgaggagctcattgtcgaggtccttgaagatgtcccgctgaccatgcacaattttgtacggaagaccttcttcagcctggcgttctgtgacttctgccttaagtttctgttccatggcttccgttgccaaacctgtggctacaagttccaccagcattgttcctccaaggtccccacagtctgtgttgacatgagtaccaaccgccaacagttctaccacagtgtccaggatttgtccggaggctccagacagcatgaggctccctcgaaccgccccctgaatgagttgctaaccccccagggtcccagcccccgcacccagcactgtgacccggagcacttccccttccctgccccagccaatgcccccctacagcgcatccgctccacgtccactcccaacgtccatatggtcagcaccacggcccccatggactccaacctcatccagctcactggccagagtttcagcactgatgctgccggtagtagaggaggtagtgatggaaccccccgggggagccccagcccagccagcgtgtcctcggggaggaagtccccacattccaagtcaccagcagagcagcgcgagcggaagtccttggccgatgacaagaagaaagtgaagaacctggggtaccgggactcaggctattactgggaggtaccacccagtgaggtgcagctgctgaagaggatcgggacgggctcgtttggcaccgtgtttcgagggcggtggcatggcgatgtggccgtgaaggtgctcaaggtgtcccagcccacagctgagcaggcccaggctttcaagaatgagatgcaggtgctcaggaagacgcgacatgtcaacatcttgctgtttatgggcttcatgacccggccgggatttgccatcatcacacagtggtgtgagggctccagcctctaccatcacctgcatgtggccgacacacgcttcgacatggtccagctcatcgacgtggcccggcagactgcccagggcatggactacctccatgccaagaacatcatccaccgagatctcaagtctaacaacatcttcctacatgaggggctcacggtgaagatcggtgactttggcttggccacagtgaagactcgatggagcggggcccagcccttggagcagccctcaggatctgtgctgtggatggcagctgaggtgatccgtatgcaggacccgaacccctacagcttccagtcagacgtctatgcctacggggttgtgctctacgagcttatgactggctcactgccttacagccacattggctgccgtgaccagattatctttatggtgggccgtggctatctgtccccggacctcagcaaaatctccagcaactgccccaaggccatgcggcgcctgctgtctgactgcctcaagttccagcgggaggagcggcccctcttcccccagatcctggccacaattgagctgctgcaacggtcactccccaagattgagcggagtgcctcggaaccctccttgcaccgcacccaggccgatgagttgcctgcctgcctactcagcgcagcccgccttgtgccttaggccccgcccaagccaccagggagccaatctcagccctccacgccaaggagccttgcccaccagccaatcaatgttcgtctctgccctgatgctgcctcaggatcccccattccccaccctgggagatgagggggtccccatgtgcttttccagttcttctggaattgggggacccccgccaaagactgagccccctgtctcctccatcatttggtttcctcttggctttggggatacttctaaattttgggagctcctccatctccaatggctgggatttgtggcagggattccactcagaacctctctggaatttgtgcctgatgtgccttccactggattttggggttcccagcaccccatgtggattttggggggtcccttttgtgtctcccccgccattcaaggactcctctctttcttcaccaagaagcacagaattctgctgggcctttgcttgtttaaaaaaaaaaaaaaaaaaaaaaaaaaa

SEQ ID NO: 4 - RNA, 3505 nt, Homo sapiens
  source 1..3505: mol_type = mRNA
cgtcaggggcaggggagggacggcgcaggcgcagaaaagggggcggcggactcggcttgttgtgttgctgcctgagtgccggagacggtcctgctgctgccgcagtcctgccagctgtccgacaatgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactgagggaaaggaaaagcccctggaaatgcgtgtgacctgtgaagtggtgtattgtcacagtagcttatttgaacttgagaccattgtaagcatgacccaacctaccaccctgtttttacatatccaattccagtaactctcaaattcaatattttattcaaactctgttgaggcattttactaaccttataccctttttggcctgaagacattttagaatttcctaacagagtttactgttgtttagaaatttgcaagggcttcttttccgcaaatgccaccagcagattataattttgtcagcaatgctattatctctaattagtgccaccagactagacctgtatcattcatggtataaattttactcttgcaacataactaccatctctctcttaaaacgagatcaggttagcaaatgatgtaaaagaagctttattgtctagttgttttttttcccccaagacaaaggcaagtttccctaagtttgagttgatagttattaaaaagaaaacaaaacaaaaaaaaaaggcaaggcacaacaaaaaaatatcctgggcaataaaaaaaatattttaaaccagctttggagccacttttttgtctaagcctcctaatagcgtcttttaatttataggaggcaaactgtataaatgataggtatgaaatagaataagaagtaaaatacatcagcagattttcatactagtatgttgtaatgctgtcttttctatggtgtagaatctttctttctgataaggaacgtctcaggcttagaaatatatgaaattgctttttgagatttttgcgtgtgtgtttgatattttttacgataattagctgcatgtgaatttttcatgaccttctttacattttttattttttatttctttatttttttttctctaagaagaggctttggaatgagttccaatttgtgatgttaatacaggcttcttgttttaggaagcatcacctatactctgaagcctttaaactctgaagagaattgtttcagagttattccaagcacttgtgcaacttggaaaaacagacttgggttgtgggaacagttgacagcgttctgaaaagatgccatttgtttccttctgatctctcactgaataatgtttactgtacagtcttcccaaggtgattcctgcgactgcaggcactggtcattttctcatgtagctgtcttttcagttatggtaaactcttaaagttcagaacactcaacagattccttcagtgatatacttgttcgttcatttctaaaatgtgaagctttaggaccaaattgttagaaagcatcaggatgaccagttatctcgagtagattttcttggatttcagaacatctagcatgactctgaaggataccacatgttttatatataaataattactgtttatgatatagacattgatattgactatttagagaaccgttgttaattttaaaactagcaatctataaagtgcaccaggtcaacttgaataaaaacactatgacagacaggtttgccagtttgcagaaactaactcttttctcacatcaacatttgtaaaattgatgtgttatagtggaaaataacatatagattaaacaaaatttttatcttttttcaagaatatagctggctatctttaagaaagatgatatatcctagttttgaaagtaattttcttttttctttctagcatttgatgtctaaataattttggacatctttttcctagaccatgtttctgtcttactcttaaacctggtaacacttgatttgccttctataacctatttatttcaagtgttcatatttgaatttctttgggaagaaagtaaatctgatggctcactgatttttgaaaagcctgaataaaattggaaagactggaaagttaggagaactgactagctaaactgctacagtatgcaatttctattacaattggtattacaggggggaaaagtaaaattacactttacctgaaagtgacttcttacagctagtgcattgtgctctttccaagttcagcagcagttctatcagtggtgccactgaaactgggtatatttatgatttctttcagcgttaaaaagaaacatagtgttgccctttttcttaaagcatcagtgaaattatggaaaattacttaaaacgtgaatacatcatcacagtagaatttattatgagagcatgtagtatgtatctgtagccctaacacatgggatgaacgttttactgctacacccagatttgtgttgaacgaaaacattgtggtttggaaaggagaattcaacaattaatagttgaaattgtgaggttaatgtttaaaaagctttacacctgtttacaatttggggacaaaaaggcaggcttcatttttcatatgtttgatgaaaactggctcaagatgtttgtaaatagaatcaagagcaaaactgcacaaacttgcacattggaaagtgcaacaagttcccgtgattgcagtaaaaatatttactattctaaaaaaatgagaattgaagacttagccagtcagataagttttttcatgaacccgttgtggaaattattggaattaactgagccaaagtgattatgcattcttcatctattttagttagcactttgtatcgttatatacagtttacaatacatgtataacttgtagctataaacattttgtgccattaaagctctcacaaaactttaaaaa

SEQ ID NO: 5 - RNA, 2949 nt, Homo sapiens
  source 1..2949: mol_type = mRNA
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 6 - RNA, 7680 nt, Homo sapiens
  source 1..7680: mol_type = mRNA
ggggccggggggcggagccttgcgggctggagcgaaagaatgcgggggctgagcgcagaagcggctcgaggctggaagaggatcttgggcgccgccagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaatctcttttccttctaaaaaaaaaaaaaaaaaaa

SEQ ID NO: 7 - RNA, 2880 nt, Homo sapiens
  source 1..2880: mol_type = mRNA
gcttccctgggtgccacggtcatgtgacttcggcaagatggctgccctgacagcggagcattttgcagcactccagagcctgctcaagctgctccaggctctgcaccgcctcactaggctggtggcattccgtgacctgtcctctgccgaggcaattctggctctctttccagaaaatttccaccaaaacctcaaaaacctgctgacaaagatcatcctagaacatgtgtctacttggagaaccgaagcccaggcaaatcagatctctctgccacgcctggtcgatctggactggagagtggatatcaaaacctcctcagacagcatcagccgcatggccgtccccacctgcctgctccagatgaagatccaagaagatcccagcctatgcggagacaaaccctccatctcagctgtcaccgtggagctgagcaaagaaacactggacaccatgttagatggcctgggccgcatccgagaccaactctctgccgtggccagtaaatgatccagccagctgccagggccactgccatgacccagctgctcatgagtgataaatgtctccccatatgcaggctgcccttgcagctgcagctgacaacaggcaggatggtggggacagcagggggctactgccatccagaagttacagttggattgggaagaagcagccagatcccccgctgttctcactcatcttctttctctttctgaagctggagagcagaagcccccatctttgaaaagctcctgagtgcaacttaattaccaccatggcagggtgagggaacatttgcatcgtcagctgcctctgcatagctgtttgagaaattcaggcccaaatcatgcagcctatccaataagtaagtttatttccaacattagctctaattagttcatttccaatcccagaacacatggagggaatcggacaggtgatgccagcagttcctgctcctctgtcagggaagccaggcagagcccacagagcatggtccatccagagtgttccctgagccccctccaccatactggaacccctcttcagtgtaggaagtctgaaatgggtgctaattcccttcttcatgaaaccagggccctcttccttcatctaatgcagccactcctaggtgaagaagtgggaataattggaaataaacaacagttctaaaacttccatgatttttgtagcttcttttgtccccaagttgaagcttttggccagtaccttctctagtttttaaagatgatcccaacttcctaattcccagctaagcccttgacccatggtgtgacatgaaatcaggcaattgaatcgcaccactttctgtgttttcacctgttacgtagaacaaaaggaagcaaggtggccaggcgcaatggctcacgcctgtaatcccagcactttgggaggccgaggcaggcagatcatgaggtcaggagatcgagaccatggtgaaaccccatctctactaaaaatacaaaaaattagctgggcgcggtggcgggcatctgtagtcccagctcctcgggaggctgaggcaggagaatggcgtgaacctgggaggcagagcttgcagtgagccgagatcgtgccactgcactccagtctgggtgacagagaaggactcgtctcaaaaaataaaaataaataaaaaggaagcaaggctaatcatcagtatgtgcttgttacaagagctatgatgaaggcactccttcgagtttaaccaaatgagatcatctctgtcatgtgcctcacgcctcacagggactccatgtgtgaagattcccccttcactcaccagatcatctccatggcaacagcttgcagcctgctcttggagtgctttgttttggcagcttctctgctagtttgtgtatggagtgaatggaggaggtaaatccacagattaagaatatgctgtcaggagtcaggcagccaaggtcagaagccagctctgcttctcagtggtaaggtgcttgacttctacatctcaattttcacccactttgtacttttttcctaaattaaatgagtataatagtagtacctacttgataggacttttgtgaaaattaaatgatataatgcacctaaaaacagtactgttacaactaataggaaaggctttgattattaatggatgagagtagaaagcttggtgcatttattgtctcatctactataacagagttggtgtgagaattagtattatcatcctccctttattgaccaggaaaccagctcattgagattgagtcatctgctggtaaatggtctcattaagaggtggacccatatttctctagctttctctttacaacacaggactttgcaaggaacatataattctgtgactagcgccatttggaaaatgttgaaactgaagtagagatgagagatcttacgtctgcctacccagtgagatacgaggaaggtcaagggaaaaaaaattccaagctcttctttatctgctataggaaatgaacattcaattttttgcatgcaacgacaagaggtcaaggaccccagaagccagcccgctacttccaagttgagagcccctggtcataccctccagttgagctcagatttgtcacaaatttacccctctcctttccttccattccccatgacctgcagagagagatgtcagataccttcctcttggcctcccatgggcatccataagaaacttacttgaagcaagaagcccagtataggtgtctgggcagttggacatttcctctagccagatctgtccgaatagagccatctgggtacatgacgcagagggcatttgataaataactggaaaagtcaataaatctttgctacccttcaaaaaaaaaaaaaaaaaaaaaaaaaaaaaaaaa

SEQ ID NO: 8 - RNA, 2358 nt, Homo sapiens
  source 1..2358: mol_type = mRNA
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 9 - RNA, 2448 nt, Homo sapiens
  source 1..2448: mol_type = mRNA
accggcccggttccctctccggggagcggcggcggacgcgcggctcccacccctcccctctcacgggctctcccctccccagtgtggccgcgaccctaccctctgcaaggcgatggcccgcgccccgagcgcaggctagcgtgcctgggtgcccggccatgggctgtatcggctctcggagcccggcgggtcaggcatttctggggaccaacagctggccgaggctcagggatagagacggctgctccagctaaaggtgaatgttggagacacagtcgcgatgctgcccaagtcccggcgagccctaactatccaggagatcgctgcgctggccaggtcctccctgcatggtatttcccaggtggtgaaggaccacgtgaccaagcctaccgccatggcccagggccgagtggctcacctcattgagtggaagggctggagcaagccgagtgactcacctgctgccctggaatcagccttttcctcctattcagacctcagcgagggcgaacaagaggctcgctttgcagcaggagtggctgagcagtttgccatcgcggaagccaagctccgagcatggtcttcggtggatggcgaggactccactgatgactcctatgatgaggactttgctgggggaatggacacagacatggctgggcagctgcccctggggccgcacctccaggacctgttcaccggccaccggttctcccggcctgtgcgccagggctccgtggagcctgagagcgactgctcacagaccgtgtccccagacaccctgtgctctagtctgtgcagcctggaggatgggttgttgggctccccggcccggctggcctcccagctgctgggcgatgagctgcttctcgccaaactgccccccagccgggaaagtgccttccgcagcctgggcccactggaggcccaggactcactctacaactcgcccctcacagagtcctgcctttcccccgcggaggaggagccagccccctgcaaggactgccagccactctgcccaccactaacgggcagctgggaacggcagcggcaagcctctgacctggcctcttctggggtggtgtccttagatgaggatgaggcagagccagaggaacagtgacccacatcatgcctggcagtggcatgcatcccccggctgctgccaggggcagagcctctgtgcccaagtgtgggctcaaggctcccagcagagctccacagcctagagggctcctgggagcgctcgcttctccgttgtgtgttttgcatgaaagtgtttggagaggaggcaggggctgggctgggggcgcatgtcctgcccccactcccggggcttgccgggggttgcccggggcctctggggcatggctacagctgtggcagacagtgatgttcatgttcttaaaatgccacacacacatttcctcctcggataatgtgaaccactaagggggttgtgactgggctgtgtgagggtggggtgggagggggcccagcaaccccccaccctccccatgcctctctcttctctgcttttcttctcacttccgagtccatgtgcagtgcttgatagaatcacccccacctggaggggctggctcctgccctcccggagcctatgggttgagccgtccctcaagggcccctgcccagctgggctcgtgctgtgcttcattcacctctccatcgtctctaaatcttcctcttttttcctaaagacagaaggtttttggtctgttttttcagtcggatcttctcttctctgggaggctttggaatgatgaaagcatgtaccctccacccttttcctggccccctaatggggcctgggccctttcccaacccctcctaggatgtgcgggcagtgtgctggcgcctcacagccagccgggctgcccattcacgcagagctctctgagcgggaggtggaagaaaggatggctctggttgccacagagctgggacttcatgttcttctagagagggccacaagagggccacaggggtggccgggagttgtcagctgatgcctgctgagaggcaggaattgtgccagtgagtgacagtcatgagggagtgtctcttcttggggaggaaagaaggtagagcctttctgtctgaatgaaaggccaaggctacagtacagggccccaccccagccagggtgttaatgcccacgtagtggaggcctctggcagatcctgcattccaaggtcactggactgtacgtttttatggttgtgggaagggtgggtggctttagaattaagggccttgtaggctttggcaggtaagagggcccaaggtaagaacgagagccaacgggcacaagcattctatatataagtggctcattaggtgtttattttgttctatttaagaatttgttttattaaattaatataaaaatctttgtaaatctctaaaaaaaaaaaaaaaaaa

SEQ ID NO: 10 - RNA, 5948 nt, Homo sapiens
  source 1..5948: mol_type = mRNA
ggagcgggccgagccgccaccgcggccggagctgtcccttagccagacccggcgagacacgagcggcgggagggaggcggtggcgcgcccggccccgcccgcccgaccaagcgtcggacgcggcccggcgccgagccatggagcctgagccagtggaggactgtgtgcagagcactctcgccgccctgtatccaccctttgaggcaacagcccccaccctgttgggccaggtgttccaggtggtggagaggacttatcgggaggacgcactgaggtacacgctggacttcctggtaccagccaagcacctgcttgccaaggtccagcaggaagcctgtgcccaatacagtggattcctcttcttccatgaggggtggccgctctgcctgcatgaacaggtggtggtgcagctagcagccctaccctggcaactgctgcgcccaggagacttctatctgcaggtggtgccctcagctgcccaagcaccccgactagcactcaagtgtctggcccctgggggtgggcgggtgcaggaggttcctgtgcccaatgaggcttgtgcctacctattcacacctgagtggctacaaggcatcaacaaggaccggccaacaggtcgcctcagtacctgcctactgtctgcgccctctgggattcagcggctgccctgggctgagctcatctgtccacgatttgtgcacaaagagggcctcatggttggacatcagccaagtacactgcccccagaactgccctctggacctccagggcttcccagccctccacttcctgaggaggcgctgggtacccggagtcctggggatgggcacaatgcccctgtggaaggacctgagggcgagtatgtggagctgttagaggtgacgctgcccgtgagggggagcccaacagatgctgaaggctccccaggcctctccagagtccggacggtacccacccgcaagggcgctggagggaagggccgccaccggagacaccgggcgtggatgcaccagaagggcctggggcctcggggccaggatggagcacgcccacccggcgaggggagcagcaccggagcctcccctgagtctcccccaggagctgaggctgtcccagaggcagcagtcttggaggtgtctgagcccccagcagaggctgtgggagaagcctccggatcttgccccctgaggccaggggagcttagaggaggaggaggaggaggccagggggctgaaggaccacctggtacccctcggagaacaggcaaaggaaacagaagaaagaagcgagctgcaggtcgaggggctcttagccgaggaggggacagtgccccactgagccctggggacaaggaagatgccagccaccaagaagcccttggcaatctgccctcaccaagtgagcacaagcttccagaatgccacctggttaaggaggaatatgaaggctcagggaagccagaatctgagccaaaagagctcaaaacagcaggcgagaaagagcctcagctctctgaagcctgtgggcctacagaagagggggccggagagagagagctggaggggccaggcctgctgtgtatggcaggacacacaggcccagaaggccccctgtctgacactccaacacctccgctggagactgtgcaggaaggaaaaggggacaacattccagaagaggcccttgcagtctccgtctctgatcaccctgatgtagcttgggacttgatggcatctggattcctcatcctgacgggaggggtggaccagagtgggcgagctctgctgaccattaccccaccgtgccctcctgaggagcccccaccctcccgagacacgctgaacacaactcttcattacctccactcactgctcaggcctgatctacagacactggggctgtccgtcctgctggaccttcgtcaggcacctccactgcctccagcactcattcctgccttgagccaacttcaggactcaggagatcctccccttgttcagcggctgctgattctcattcatgatgaccttccaactgaactctgtggatttcagggtgctgaggtgctgtcagagaatgatctgaaaagagtggccaagccagaggagctgcagtgggagttaggaggtcacagggacccctctcccagtcactgggtagagatacaccaggaagtggtaaggctatgtcgcctgtgccaaggtgtgctgggctcggtacggcaggccattgaggagctggagggagcagcagagccagaggaagaggaggcagtgggaatgcccaagccactgcagaaggtgctggcagatccccggctgacggcactgcagagggatgggggggccatcctgatgaggctgcgctccactcccagcagcaagctggagggccaaggcccagctacactgtatcaggaagtggacgaggccattcaccagcttgtgcgcctctccaacctgcacgtgcagcagcaagagcagcggcagtgcctgcggcgactccagcaggtgttgcagtggctctcgggcccaggggaggagcagctggcaagctttgctatgcctggggacaccttgtctgccctgcaggagacagagctgcgattccgtgctttcagcgctgaggtccaggagcgcctggcccaggcacgggaggccctggctctggaggagaatgccacctcccagaaggtgctggatatctttgaacagcggctggagcaggttgagagtggcctccatcgggccctgcggctacagcgcttcttccagcaggcacatgaatgggtggatgagggctttgctcggctggcaggagctgggccgggtcgggaggctgtgctggctgcactggccctgcggcgggccccagagcccagtgccggcaccttccaggagatgcgggccctggccctggacctgggcagcccagcagccctgcgagaatggggccgctgccaggcccgctgccaagagctagagaggaggatccagcaacacgtgggagaggaggcgagcccacggggctaccgacgacggcgggcagacggtgccagcagtggaggggcccagtgggggccccgcagcccctcgcccagcctcagctccttgctgctccccagcagccctgggccacggccagccccatcccattgctccctggccccatgtggagaggactatgaggaagagggccctgagctggctccagaagcagagggcaggcccccaagagctgtgctgatccgaggcctggaggtcaccagcactgaggtggtagacaggacgtgctcaccacgggaacacgtgctgctgggccgggctagggggccagacggaccctggggagtaggcaccccccggatggagcgcaagcgaagcatcagtgcccagcagcggctggtgtctgagctgattgcctgtgaacaagattacgtggccaccttgagtgagccagtgccaccccctgggcctgagctgacgcctgaacttcggggcacctgggctgctgccctgagtgcccgggaaaggcttcgcagcttccaccggacacactttctgcgggagcttcagggctgcgccacccaccccctacgcattggggcctgcttccttcgccacggggaccagttcagcctttatgcacagtacgtgaagcaccgacacaaactggagaatggtctggctgcgctcagtcccttaagcaagggctccatggaggctggcccttacctgccccgagccctgcagcagcctctggaacagctgactcggtatgggcggctcctggaggagctcctgagggaagctgggcctgagctcagttctgagtgccgggcccttggggctgctgtacagctgctccgggaacaagaggcccgtggcagagacctgctggccgtggaggcggtgcgtggctgtgagatagatctgaaggagcagggacagctcttgcatcgagaccccttcactgtcatctgtggccgaaagaagtgccttcgccatgtctttctcttcgagcatctcctcctgttcagcaagctcaagggccctgaaggggggtcagagatgtttgtttacaagcaggcctttaagactgctgatatggggctgacagaaaacatcggggacagcggactctgctttgagttgtggtttcggcggcggcgtgcacgagaggcatacactctgcaggcaacctcaccagagatcaaactcaagtggacaagttctattgcccagctgctgtggagacaggcagcccacaacaaggagctccgagtgcagcagatggtgtccatgggcattgggaataaacccttcctggacatcaaagcccttggggagcggacgctgagtgccctgctcactggaagagccgcccgcacccgggcctccgtggccgtgtcatcctttgagcatgccggcccctcccttcccggcctttcgccgggagcctgctccctgcctgcccgcgtcgaggaggaggcctgggatctggacgtcaagcaaatttccctggccccagaaacacttgactcttctggagatgtgtccccaggaccaagaaacagccccagcctgcaacccccccaccctgggagcagcactcccaccctggccagtcgagggatcttagggctatcccgacagagtcatgctcgagccctgagtgaccccaccacgcctctgtgacctggagaagatccagaacttgcgtgcagcttctcctctcagcacactttgggctgggatggcagtggggcataatggagccctgggcgatcgctgaatttcttccctctgcttcctggacacagaggaggtctaacgaccagagtattgccctgccaccactatctctagtctccctagcttggtgccttctcctgcaggagtcagagcagccacattgcttgccttcataccctggaggtggggaagttatccctcttccggtgctttcccatcctgggccactgtatccaggacatcactcccatgccagccctccctggcagcccatgttctcctcttttctcaccccctgactttccctgagaagaatcatctctgccaggtcaactggagtccctggtgactccattctgaggtgtcacaagcaatgaagctatgcaaacaataggagggtgtgacaggggaaccgtagactttatatatgtaattactgttattataatactattgttatattaaatgtatttactcacactttgcctctaaggagctagagtagtcctctggattaaggtgataaataacttgagcactttccctcaaccagcccttaactagaacacagaaaataaaaccaagactggaaggtcccctctacccctcccaggcccagagctagctgactgtgtatgagcctgggagaatgtgtctcctccacagtggctcccagaggttccacacactctctgaagctccttctcccacactgcacctactccttgaggctgaactggtcacagacaaactgggatccagcacagtccagcagttctcaaaatgaggtcctcaggccacagtgcgtgagaacttgcttggctgtttgttaaatgctaattcttgggccccatcagagctactgcatcgaaacctgggggtaaaacccaatattctgcatttcttatcaaactctttgggtgataactaagtgtctgaagaggtgactatttcctgacagaaggacccaaagagggaagcaggacataggtaggcagacagacacagggccctgtgcctcaagacacctgtttattggggacacgactctgcaatagggatgacaggaatcgtaccaaaaatagcgacgtctacagggcccctgatggggctagaagggtacagtgccccccaccctcaccccttgtacaaaaataaactctcacgcctatggaccagcaaaaaaaaaaaaaa

SEQ ID NO: 11 - RNA, 3851 nt, Homo sapiens
  source 1..3851: mol_type = mRNA
ctctcccaaccgcctcgtcgcactcctcaggctgagagcaccgctgcactcgcggccggcgatgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatgagccccggcccctccccacctttcccaccccagccctcttgcaagaggagaggcacggtagggaaaagaactgctgggtgggggcctgtttctgtaactttctccccctctactgagaagtgacctggaagtgagaagttctttgcagatttggggcgaggggtgatttggaaaagaagacctgggtggaaagcggtttggatgaaaagatttcaggcaaagacttgcaggaagatgatgataacggcgatgtgaatcgtcaaaggtacgggccagcttgtgcctaatagaaggttgagaccagcagagactgctgtgagtttctcccggctccgaggctgaacggggactgtgagcgatccccctgctgcagggcgagtggcctgtccagacccctgtgaggccccgggaaaggtacagccctgtctgcggtggctgctttgttggaaagagggagggcctcctgcggtgtgcttgtcaagcagtggtcaaaccataatctcttttcactggggccaaactggagcccagatgggttaatttccagggtcagacattacggtctctcctcccctgccccctcccgcctgtttttcctcccgtactgctttcaggtcttgtaaaataagcatttggaagtcttgggaggcctgcctgctagaatcctaatgtgaggatgcaaaagaaatgatgataacattttgagataaggccaaggagacgtggagtaggtatttttgctactttttcattttctggggaaggcaggaggcagaaagacgggtgttttatttggtctaataccctgaaaagaagtgatgacttgttgcttttcaaaacaggaatgcatttttccccttgtctttgttgtaagagacaaaagaggaaacaaaagtgtctccctgtggaaaggcataactgtgacgaaagcaacttttataggcaaagcagcgcaaatctgaggtttcccgttggttgttaatttggttgagataaacattcctttttaaggaaaagtgaagagcagtgtgctgtcacacaccgttaagccagaggttctgacttcgctaaaggaaatgtaagaggttttgttgtctgttttaaataaatttaattcggaacacatgatccaacagactatgttaaaatattcagggaaatctctcccttcatttactttttcttgctataagcctatatttaggtttcttttctatttttttctcccatttggatcctttgaggtaaaaaaacataatgtcttcagcctcataataaaggaaagttaattaaaaaaaaaaagcaaagagccattttgtcctgttttcttggttccatcaatctgtttattaaacatcatccatatgctgaccctgtctctgtgtggttgggttgggaggcgatcagcagataccatagtgaacgaagaggaaggtttgaaccatgggccccatctttaaagaaagtcattaaaagaaggtaaacttcaaagtgattctggagttctttgaaatgtgctggaagacttaaatttattaatcttaaatcatgtactttttttctgtaatagaactcggattcttttgcatgatggggtaaagcttagcagagaatcatgggagctaacctttatcccacctttgacactaccctccaatcttgcaacactatcctgtttctcagaacagtttttaaatgccaatcatagagggtactgtaaagtgtacaagttactttatatatgtaatgttcacttgagtggaactgctttttacattaaagttaaaatcgatcttgtgtttcttcaaccttcaaaactatctcatctgtcagatttttaaaactccaacacaggttttggcatcttttgtgctgtatcttttaagtgcatgtgaaatttgtaaaatagagataagtacagtatgtatattttgtaaatctcccatttttgtaagaaaatatatattgtatttatacatttttactttggatttttgttttgttggctttaaaggtctaccccactttatcacatgtacagatcacaaataaatttttttaaatac

SEQ ID NO: 12 - RNA, 2394 nt, Homo sapiens
  source 1..2394: mol_type = mRNA
gacgggccggtacagcccgtgtccccgccccgcgccatcgctaggcgacgtgcgcttttgccgcgccgtgctgcccgcgagggcagctgaggtggtggtggcggccgccttgtcgaggcatcgcgcgcccgtgaagtgttcgccgtcagtgctgttgggtgcctggagccgcgtcccccgtcccgaaaactgtccttgacagtacttgcgcggcccaacggccgccggcgcccccgcgtctccatggcgacggcctttttccctgcgaggaccccggcggcagggctgccccgcggcgcctgcttggcgcgacgctctagcggttaccgctgcgggctggctgggcgtagtggggctgcgcggctgccacggagctagagggcaagtgtgctcggcccagcgtgcagggaacgcgggcggccagacaacgggctgggctccggggcctgcggcgcgggcgctgagctggcagggcgggtcggggcgcgggctgcatccgcatctcctccatcgcctgcagtaagggcggccgcggcgagcctttgaggggaacgacttgtcggagccctaaccaggggtatctctgagcctggtgggatccccggagcgtcacatcactttccgatcacttcaaagtacagcagaccgaggacacggttgttaccaagaccaggctgttgccttggaagagcccagagcgtgtcaagggagacagccacatcacgccagaaatacatgacagctggattagccctgggagagggaggcccagatgtgggagctcaggggaggtgcagctcaacgtggagtttggaggaggctaccttgacctttgaatgccaagtgggagccagccagatgaaaggggttaaaaactaatatttatatgacagaagaaaaagatgtcattccgtaaagtaaacatcatcatcttggtcctggctgttgctctcttcttactggttttgcaccataacttcctcagcttgagcagtttgttaaggaatgaggttacagattcaggaattgtagggcctcaacctatagactttgtcccaaatgctctccgacatgcagtagatgggagacaagaggagattcctgtggtcatcgctgcatctgaagacaggcttgggggggccattgcagctataaacagcattcagcacaacactcgctccaatgtgattttctacattgttactctcaacaatacagcagaccatctccggtcctggctcaacagtgattccctgaaaagcatcagatacaaaattgtcaattttgaccctaaacttttggaaggaaaagtaaaggaggatcctgaccagggggaatccatgaaacctttaacctttgcaaggttctacttgccaattctggttcccagcgcaaagaaggccatatacatggatgatgatgtaattgtgcaaggtgatattcttgccctttacaatacagcactgaagccaggacatgcagctgcattttcagaagattgtgattcagcctctactaaagttgtcatccgtggagcaggaaaccagtacaattacattggctatcttgactataaaaaggaaagaattcgtaagctttccatgaaagccagcacttgctcatttaatcctggagtttttgttgcaaacctgacggaatggaaacgacagaatataactaaccaactggaaaaatggatgaaactcaatgtagaagagggactgtatagcagaaccctggctggtagcatcacaacacctcctctgcttatcgtattttatcaacagcactctaccatcgatcctatgtggaatgtccgccaccttggttccagtgctggaaaacgatattcacctcagtttgtaaaggctgccaagttactccattggaatggacatttgaagccatggggaaggactgcttcatatactgatgtttgggaaaaatggtatattccagacccaacaggcaaattcaacctaatccgaagatataccgagatctcaaacataaagtgaaacagaatttgaactgtaagcaagcatttctcaggaagtcctggaagatagcatgcgtgggaagtaacagttgctaggcttcaatgcctatcggtagcaagccatggaaaaagatgtgtcagctaggtaaagatgacaaactgccctgtctggcagtcagcttcccagacagactatagactataaatatgtctccatctgccttaccaagtgttttcttactacaatgctgaatgactggaaagaagaactgatatggctagttcagctagctggtacagataattcaaaactgctgttggttttaattttgtaacctgtggcctgatctgtaaataaaacttacatttttcaataggtaaaaaaaaaaaaaaaa

SEQ ID NO: 13 - RNA, 5765 nt, Homo sapiens
  source 1..5765: mol_type = mRNA
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 14 - RNA, 2202 nt, Homo sapiens
  source 1..2202: mol_type = mRNA
cgtaaagagaggccgggagctgcccctaaccgaggcagcagcggacgtgagcgataatggcggatatggaggatctcttcgggagcgacgccgacagcgaagctgagcgtaaagattctgattctggatctgactcagattctgatcaagagaatgctgcctctggcagtaatgcctctggaagtgaaagtgatcaggatgaaagaggtgattcaggacaaccaagtaataaggaactgtttggagatgacagtgaggacgagggagcttcacatcatagtggtagtgataatcactctgaaagatcagacaatagatcagaagcttctgagcgttctgaccatgaggacaatgacccctcagatgtagatcagcacagtggatcagaagcccctaatgatgatgaagacgaaggtcatagatcggatggagggagccatcattcagaagcagaaggttctgaaaaagcacattcagatgatgaaaaatggggcagagaagataaaagtgaccagtcagatgatgaaaagatacaaaattctgatgatgaggagagggcacaaggatctgatgaagataagctgcagaattctgacgatgatgagaaaatgcagaacacagatgatgaggagaggcctcagctttccgatgatgagagacaacagctatctgaggaggaaaaggctaattctgatgatgaacggccggtagcttctgataatgatgatgagaaacagaattctgatgatgaagaacaaccacagctgtctgatgaagagaaaatgcaaaattctgatgatgaaaggccacaggcctcagatgaagaacacaggcattcagatgatgaagaggaacaggatcataaatcagaatctgcaagaggcagtgatagtgaagatgaagttttacgaatgaaacgcaagaatgcgattgcatctgattcagaagcggatagtgacactgaggtgccaaaagataatagtggaaccatggatttatttggaggtgcagatgatatctcttcagggagtgatggagaagacaaaccacctactccaggacagcctgttgatgaaaatggattgcctcaggatcaacaggaagaggagccaattcctgagaccagaatagaagtagaaatacccaaagtaaacactgatttaggaaacgacttatattttgttaaactgcccaactttctcagtgtagagcccagaccttttgatcctcagtattatgaagatgaatttgaagatgaagaaatgctggatgaagaaggtagaaccaggttaaaattaaaggtagaaaatactataagatggaggatacgccgagatgaagaaggaaatgaaattaaagaaagcaatgctcggatagtcaagtggtcagatggaagcatgtccctgcatttaggcaatgaagtgtttgatgtgtacaaagccccactgcagggcgaccacaatcatctttttataagacaaggtactggtctacagggacaagcagtctttaaaacgaaactcaccttcagacctcactctacggacagtgccacacatagaaagatgactctgtcacttgcagataggtgttcaaagacacagaagattagaatcttgccaatggctggtcgtgatcctgaatgccaacgcacagaaatgattaagaaagaagaagaacgtttgagggcttccatacgtagggaatctcagcagcgccgaatgagagagaaacagcaccagcgggggctgagcgccagttacctggaacctgatcgatacgatgaggaggaggaaggcgaggagtccatcagcttggctgccattaaaaaccgatataaagggggcattcgagaggaacgagccagaatctattcatcagacagtgatgagggatcagaagaagataaagctcaaagattactcaaagcaaagaaacttaccagtgatgaggaaggtgaaccttccggaaagagaaaagcagaagatgatgataaagcaaataaaaagcataagaagtatgtgatcagcgatgaagaggaagaagatgatgattgaagtatgaaatatgaaaacattttatatattttattgtacagttataaatatgtaaacatgagttattttgattgaaatgaatcgatttgcttttgtgtaattttaattgtaataaaacaatttaaaagcaaaaaaaaaaaaaaaa

SEQ ID NO: 15 - RNA, 2047 nt, Homo sapiens
  source 1..2047: mol_type = mRNA
ttgattatggaacattctaaaacttagacaagacgattgtgattggctgaagggcatacgccctcctccagggtgacgtgtctgcctatggatatcagttgccagagaaacctggctttactatggcggttggaggaacggcagtgatcacacgtcggctgctgggaagatctggattctcgtttcaggtcaccatcagaaaagctaagtttgctgtatagtgaggatcaggagatctgatcctgattgcagaaccttccctgattacagaatcttgggattgttgagaggattacatgtaaagtaccaggacagtgcatggcacatatgatttcacaaaagttcatcttcattgcagatacctgcctttctttctaggttgtatctcccacttcacccttctagaccatcccagaagatctataagatttcatctgggaaatcactaggagttcttggaagggaaagaaggaagattgttggttggaataaaaacagggttgaatgagttccagaaagcagggttctcaacctcgtggacagcaatctgcagaagaagagaacttcaaaaaaccaactagaagcaacatgcagagaagtaaaatgagaggggcctcctcaggaaagaagacagctggtccacagcagaaaaatcttgaaccagctctcccaggaagatggggtggtcgctctgcagagaaccccccttcaggatccgtgaggaagaccagaaagaacaagcagaagactcctggaaacggagatggtggcagtaccagcgaagcacctcagccccctcggaagaaaagggcccgggcagaccccactgttgaaagtgaggaggcgtttaagaatagaatggaggttaaagtgaagattcctgaagaattaaaaccatggcttgttgaggactgggacttagttaccaggcagaagcagctgtttcaactccctgccaagaaaaatgtagatgcaattctggaggagtatgcaaattgcaagaaatcgcagggaaatgttgataataaggaatatgcggttaatgaagttgtggcaggaataaaagaatatttcaatgtgatgttgggcactcagctgctctacaaatttgagaggccccagtatgctgaaatcctcttggctcaccctgatgctccaatgtcccaggtttatggagcaccacacctactgagattatttgtaagaattggagcaatgttggcctatacgccccttgatgagaaaagccttgcattattgttgggctatttgcatgatttcctaaaatatctggcaaagaattctgcatctctctttactgccagtgattacaaagtggcttctgctgagtaccaccgcaaagccctgtgagcgtctacagacagctcaccatttttgtcctgtatctgtaaacactttttgttcttagtctttttcttgtaaaattgatgttctttaaaatcgttaatgtataacagggcttatgtttcagtttgttttccgttctgttttaaacagaaaataaaaggagtgtaagctccttttctcatttcaaagttgctaccagtgtatgcagtaattagaacaaagaagaaacattcagtagaacattttattgcctagttgacaacattgcttgaatgctggtggttcctatccctttgacactacacaattttctaatatgtgttaatgctatgtgacaaaacgccctgattcctagtgccaaaggttcaacttaatgtatatacctgaaaacccatgcatttgtgctctttttttttttttatggtgcttgaagtaaaacagcccatcctctgcaagtccatctatgttgttcttaggcattctatctttgctcaaattgttgaaggatggtgatttgtttcatggtttttgtatttgagtctaatgcacgttctaacatgatagaggcaatgcattattgtgtagccacggttttctggaaaagttgatattttaggaattgtatttcagatcttaaataaaatttgtttctaaatttcaaagcaaaaaaaaaaaaaaa

SEQ ID NO: 16 - RNA, 2410 nt, Homo sapiens
  source 1..2410: mol_type = mRNA
ggtgcagccttacgccgctgacgcatcgcgcccaagatggcggcgcggtcgtcgtcgggggtggcggcggcagagggggcggcggccctggcggcagcggagacggcagccgtgacggtggcagcggcggcgcgggacctgggcctgggggaatgaggcggccgcggcgggccagcggcggagccgtgtagcggagaagctccccctccctgcttcccttggccgagccgggggcgcgcgcgcacgcggccgtccagagcgggctccccacccctcgactcctgcgacccgcaccgcacccccacccgggcccggaggatgatgaagctcaagtcgaaccagacccgcacctacgacggcgacggctacaagaagcgggccgcatgcctgtgtttccgcagcgagagcgaggaggaggtgctactcgtgagcagtagtcgccatccagacagatggattgtccctggaggaggcatggagcccgaggaggagccaagtgtggcagcagttcgtgaagtctgtgaggaggctggagtaaaagggacattgggaagattagttggaatttttgagaaccaggagaggaagcacaggacgtatgtctatgtgctcattgtcactgaagtgctggaagactgggaagattcagttaacattggaaggaagagggaatggtttaaaatagaagacgccataaaagtgctgcagtatcacaaacccgtgcaggcatcatattttgaaacattgaggcaaggctactcagccaacaatggcaccccagtcgtggccaccacatactcggtttctgctcagagctcgatgtcaggcatcagatgactgaagacttcctgtaagagaaatggaaattggaaactagactgaagtgcaaatcttccctctcaccctggctctttccacttctcacaggcctcctctttcaaataaggcatggtgggcagcaaagaaagggtgtattgataatgttgctgtttggtgttaagtgatggggctttttcttctgtttttattgagggtgggggttgggtgtgtaatttgtaagtacttttgtgcatgatctgtccctccctcttcccacccctgcagtcctctgaagagaggccaacagccttcccctgccttggattctgaagtgttcctgtttgtcttatcctggccctggccagacgttttctttgatttttaattttttttttttattaaaagataccagtatgagatgaaaacttccaataatttgtcctataatgtgctgtacagttcagtagagtggtcactttcactgcagtatacatttatctacacattatatatcggacatataatatgtaaataaatgacttctagaaagagaaatttgtttaatttttcaaggtttttttctcttttaatttgggcatttctagaattgagagcctcacaattaacatacctttttgttttcgatgctagtggctgggcaggttgccctgtcctttctctatttcccagtcattgactgtagatatgggaagagtttagctaccttcatagtgctcccaggactcatggcctttccttctttaagctgtatttccctgcccagaaagaaacaggaagaaacctttttttatttttttatttttttttaaccaagcaaggagcaaatggcctcagcccagatctgtaaaaacaatgatagaaattgaattctgccccacatgttgacagtagagttggaactggattcttgggattacttatctaaaaaactggagcatcaggtccatttctgttctgctggtttggaatcttttccgtaatgctatttattgccaacaatggcctctctttgtgtccatatatgccttacaccgtgctgacctgggtatcatccatgtgctctgaagcatccaactttactttgcaggtgcatcaatgtagtcctgtccctgaactgagtaaccgtgttcctgaaaagtacactagggaaattcacctgcttgcttgtctttgtattggcatggcacttgtgattgcaccatggagcatgctcagagctattaaattggtctcccatctcccaccaggatatgaaaggtccatatgggaggccacgtaatcacttattacagtggttacataatacactggctcactgcagactctcttgttttttgatacagtttcgtgctggcttcatttgccaattgtgttgtttagttcggaagtaagagggtcttgagattgaggggtagggagggctacactgactgatccgtggcttaagacaggagattatctctgtactccagtggcatctccttagccaagatgtgaaattaaaatcatagttcgcctcatttaaaaattctaataaagcactcaaactttgaaaaaaaaaaaaaaaaaa

SEQ ID NO: 17 - RNA, 1947 nt, Homo sapiens
  source 1..1947: mol_type = mRNA
atgcagatgaggcactcgggggcggggcggcggcggcggcggcggcggtggcggccggggagggtcagttggaggcaggcgctcgctgaggcaaaaggaggcgctcggcccgcggcctgacagggacttagcccgcagagatcgaccccgcgcgcgtgaccccacacccacccactcatccatctatccactccctgcgccgcctcctcccaccctgagcagagccgccgaggatgataaacacccaggacagtagtattttgcctttgagtaactgtccccagctccagtgctgcaggcacattgttccagggcctctgtggtgctcctgatgcccctcacccactgtcgaagatccccggtgggcgagggggcggcagggatccttctctctcagctctaatatataaggacgagaagctcactgtgacccaggacctccctgtgaatgatggaaaacctcacatcgtccacttccagtatgaggtcaccgaggtgaaggtctcttcttgggatgcagtcctgtccagccagagcctgtttgtagaaatcccagatggattattagctgatgggagcaaagaaggattgttagcactgctagagtttgctgaagagaagatgaaagtgaactatgtcttcatctgcttcaggaagggccgagaagacagagctccactcctgaagaccttcagcttcttgggctttgagattgtacgtccaggccatccctgtgtcccctctcggccagatgtgatgttcatggtttatcccctggaccagaacttgtccgatgaggactaatagtcatagaggatgctttacccaagagccacagtgggggaagaggggaagttaggcagccctgggacagacgagagggctcctcgctgtctagggaaggacactgaggggctcagggtgagggttgcctattgtgttctcggagttgactcgttgaaattgttttccataaagaacagtataaacatattattcacatgtaatcaccaatagtaaatgaagatgtttatgaactggcattagaagctttctaaactgcgctgtgtgatgtgttctatctagcctaggggaggacattgcctagagggggagggactgtctgggttcaggggcatggcctggagggctggtgggcagcactgtcaggctcaggtttccctgctgttggctttctgttttggttattaagacttgtgtattttctttctttgcttcctgtcaccccaggggctcctgagtataggcttttcagtccctgggcagtgtccttgagttgttttttgacactcttacctgggcttctctgtgtgcatttgcgtctggcctggagtaagcaggtccgacccctccttctttacagcttagtgttattctggcatttggttaagctggcttaatctgtttaatgttatcagtacattttaaataggggcattgaaatttactcccaccaccagggcttttttgggggatgcctgggcctttaaaacactagccaaactctaattaattctcaaatcactgccaggagttcttgctcctggctgcaggcccaggccccaaggtctccttcttggggtcacaaacagcagtaaggaagaggaatatatagcaactcagggcctgggaattgtggggcaatccgttcttagggactggatacttctggctggctgagtatagtactagctgcctccccaccaggttccgagtagtgtctgagactctgctctgcagggcctagggtagcgctgggagtgtagaagtggcctgcccttaactgttttcactaaacagctttttctaaggggagagcaagggggagagatctagattgggtgagggggacggggatgtcagggaggcaagtgtgttgtgttactgtgtcaataaactgatttaaagttgtgaaaaaaaaaaaaa

SEQ ID NO: 18 - RNA, 1712 nt, Homo sapiens
  source 1..1712: mol_type = mRNA
atgctgggggaggggctggcggcctcgacggcagctgcggaactaggccgagggacaaaggctaagtttttccatggtttggactggatatcggtggaactctggtcaagctggtatattttgaacccaaagacatcactgctgaagaagaagaggaagaagtggaaagtcttaaaagcattcggaagtacctgacctccaatgtggcttatgggtctacaggcattcgggacgtgcacctcgagctgaaggacctgactctgtgtggacgcaaaggcaatctgcactttatacgctttcccactcatgacatgcctgcttttattcaaatgggcagagataaaaacttctcgagtctccacactgtcttttgtgccactggaggtggagcgtacaaatttgagcaggattttctcacaataggtgatcttcagctttgcaaactggatgaactagattgcttgatcaaaggaattttatacattgactcagtcggattcaatggacggtcacagtgctattactttgaaaaccctgctgattctgaaaagtgtcagaagttaccatttgatttgaaaaatccgtatcctctgcttctggtgaacattggctcaggggttagcatcttagcagtatattccaaagataattacaaacgggtcacaggtactagtcttggaggaggaactttttttggtctctgctgtcttcttactggctgtaccacttttgaagaagctcttgaaatggcatctcgtggagatagcaccaaagtggataaactagtacgagatatttatggaggggactatgagaggtttggactgccaggctgggctgtggcttcaagctttggaaacatgatgagcaaggagaagcgagaggctgtcagtaaagaggacctggccagagcgactttgatcaccatcaccaacaacattggctcaatagcaagaatgtgtgcccttaatgaaaacattaaccaggtggtatttgttggaaatttcttgagaattaatacgatcgccatgcggcttttggcatatgctttggattattggtccaaggggcagttgaaagcacttttttcggaacacgagggttattttggagctgttggagcactccttgagctgttgaagatcccgtgatcattacctggggaggggttcctgaaaccttccacaatgggatctgtggactttcatttttttaagagacttactcaatttcatgactgtactacctgaaacaaagtgagaaaggacaggtgtatttttctaagtcatcaagataaatccttaagaattcagtctaaattagcaaccaggaaggaaaaatatattaaaaacaacaaaaaagtggcacatgtccaggcagtgtgaggatttgctgtatataagttgcctgctttgtatttttgaaatctctgcatcactcattggaagtgcttctgaagagagctgctctgtgttcagttgactggttttgtgtcctgtttgaacttgctgaatgtaaggcaggctactatgcgttataatctaatcacaatttgtcaatatggtcttggcaatcatctgtgcattactctggtttgcattaagcctgtgtgtgaacttactgtaaaacatgttttatttcaaggttctgcaaaattaattgggcaggttaattgtgtacctgaaacttaacaagcagtttttggaagggca

SEQ ID NO: 19 - RNA, 7332 nt, Homo sapiens
  source 1..7332: mol_type = mRNA
ggtgaatgggctggtggtgctcgctgctgctgctgagaggaggaggaggatgaagagttgggcttgtttgtctcctacagtttctctcctgctgctctgattcccccctcccgattccggcccggggcctgtgtgtgtccctcctggaggaggaggaggatccagttcctccccccaaccccctcctccccacccccccttgcctggggaagaggaggaaagaaacagcccagagagagagagagagagagagtgagtgagagagagaggagaggagaggaggaggaggaggagggagaagggaacaacctaccatcttaacacactaatatctaaaaagtgcgagaggcccagagcagcagcagaagcagcagcagcagctccagcttcttccctccctccccatgaagaagagttccctcctcctcctcctcctgcttctcctgctcagagttcctgcctccagctgccaggggggacagccagccagcagcaggaggggggctagagagctgaaggagagccagtttccccaaaattgctgcagtgagaagaggagtttgttactttaaacagaggctgaagaaactatagaattagcagagaaagtggagaaggtagaggatggagttgcagactctacaggaggctcttaaagtggaaattcaggttcaccagaaactggttgctcaaatgaagcaggatccacagaatgctgacttaaagaaacagcttcatgaactccaagccaaaatcacagctttgagtgagaaacagaaaagagtagttgaacagctacggaagaacctgatagtaaagcaagaacaaccggacaagttccaaatacagccattgccacaatctgaaaacaaactacaaacagcacagcagcaaccactacagcaactacaacaacagcagcagtaccaccaccaccacgcccagcagtcagctgcagcctctcccaacctgactgcttcacagaagactgtaactacagcttctatgattaccacaaagacactacctctcgtcttgaaagcagcaactgcgaccatgcctgcctctgtggtgggccagagacctaccattgctatggtgaccgccatcaacagtcagaaggctgtgctcagcactgatgtgcagaacacaccagtcaacctccagacgtctagtaaggtcactgggcctggggcagaggctgtccaaattgtggcaaaaaacacagtcactctggttcaggcaacacctcctcagcccatcaaagtaccacagtttatcccccctcctagactcactccacgtccaaactttcttccacaggttcgacccaagcctgtggcccagaataacattcctattgccccagcaccacctcccatgctcgcagctcctcagcttatccagaggcccgtcatgctgaccaagttcacccccacaacccttcccacatcccagaattccatccaccccgtccgtgtcgtcaatgggcagactgcaaccatagccaaaacgttccccatggcccagctcaccagcattgtgatagctactccagggaccagactcgctggacctcaaactgtacagcttagcaagccaagtcttgaaaaacagacagttaaatctcacacagaaacagatgagaaacaaacagagagccgcaccatcaccccacctgctgcacccaaaccaaaacgggaggagaaccctcagaaacttgccttcatggtgtctctagggttggtaacacatgaccatctagaagaaatccaaagcaagaggcaagagcgaaaaagaagaacaacagcaaatccggtctacagtggagcagtctttgagccagagcgtaagaagagtgcagtgacatacctaaacagcacaatgcaccctgggacccggaagagaggtcgtcctccaaaatacaatgcagtgctggggtttggagcccttaccccaacatccccccaatccagtcatcctgactcccctgaaaatgaaaagacagagaccacattcactttccctgcacctgttcagcctgtgtccctgcccagccccacctccacagacggtgatattcatgaggatttttgcagcgtttgcagaaaaagtggccagttactgatgtgcgacacatgttcccgtgtatatcatttggactgcttagacccccctctgaaaacaattcccaagggcatgtggatctgtcccagatgtcaggaccagatgctgaagaaggaagaagcaattccatggcctggaactttagcaattgttcattcctatattgcctacaaagcagcaaaagaagaagagaaacagaagttacttaaatggagttcagatttaaaacaagaacgagaacaactagagcaaaaggtgaaacagctcagcaattccataagtaaatgcatggaaatgaagaacaccatcctggcccggcagaaggagatgcacagctccctggagaaggtaaaacagctgattcgcctcatccacggcatcgacctctccaaacctgtagactctgaggccactgtgggggccatctccaatggcccggactgcaccccccctgccaatgccgccacctccacgccggccccttccccctcctcccagagctgcacagcgaactgtaaccagggggaagagactaaataacagagcccctctaggagaagccacgggatcccggcggcaaggagaacagaacactgaagactctagaaaagcaaagccggatttctggaaagtgcagaattcttttggttctttggttccagagagagagaagatgcttgtgccaggtggcaccagagtttgccaattgatccttcttattctgtgtgtacatgcaaagattggaccatgttacatgaaatagtgccagctggaggttctttgccagcaccatgccaagtgaaataatatatttactctctctattatacaccagtgtgtgcctgcagcagcctccacagccacgatgggtttgtttctgttttcttgggtggggagcagggacgggcggagggaggagagcaggtttcagatccttacttgccgagccgtttgtttaggtagagaagacaagtccaaagagtgtgtgggctttcctgtttctaaactttcgctactataaaaccaaaaaaaggaattgagatttcaccaaccccagtgcccagaagagggaaggggagtggctggagggagcagggggtgggacagtgtatcaaataagcagtatttaatcacctctggcgggggcctcgtgcaaggggagactgacaccaagaacagccagtaggttcttctcccctgcactctgctccctgcgcggtaaccccaccactcctgaagcctgcccagtctccttccttccctgcttggtgagtcgcgcatctccgtggttatcccgctgtctcctctccaagaacaagcagagcccgggccactggcccttgcccaaggcagggaagaaggatgtgtgtgtccaggaaggaaaaaaaggtggatcagtgattttacttgaaaacaagctccatcccttttctatatttataagaagagaagatcttgagtgaagcagcacgcgacccaggtgtgtgtgaattgaatggagacgtttcttttctctttctttaatttttgtttttgttctttttttctttaaggaaagttttattttactgttcattttactttcttggtaacaaaaactaaaataaggaatagaaaagctgtttttcaggctgacagtccaattaagggtagccaagaccttgcatggtagagtaggaatcatagtgtcagtgaggtcccgtgagtctttgtgagtccttgtgtcatcgttcgggcactgtttttttatgcaagggcaaaaatctttgtatctggggaaaaaaaacttttttttaaattaaaaaggaaaataaaagatattgaggtcttcctagtgttacttaaattaagatcaaggtaagaaacattgtaaaaaaaaattacaaaagtgctatttgtttcctaaaaacagtgatttctattaaaaaggtgtcagaactggagaaaatgccgtgtagttataattttttagcacagaccctgctgatcacgatgacattttgccgtgtgtgtgtctctagactggtgggccagtctccttgaaggacagaggcggagctccccacccttctctctcctcagaaaagaccgtgctctcttcttggtgcagggatcttgtctcctgttgtgaagcccaaatggaagcgtggatggtatcagggccctacccgtggtcttctcagattctgctagagcaaaaggctggtgcctaaataagatcccttcctttggtgctgcttttggtctttcagccaccagcattatgagtgcctgggggacacctccgagggaactggccagcggagctctgtggtgcgcacgcaccctggccgtgacaggagggtgcgggagtacaggctggctgcatcagcccttggtgcttagaacagaggaggagtgacatgttttgagggtacgtctctgagacagagccccagcgtggccttcgctctgtcttgcctttggggagaggtctgaagctcccactcctttctctgcctgttggctccaggcaccagaaatttactccactccacccacccacaagcctcctgggtgaccctgggctagaattgctgcgcttgcctcggcttggccggttgtggcctctccttgagaaaaccagggttgtgaaagactcagaccattctctcatcttgccttgtcagaagtaaattgtgtcagatttgtgctctcgctggagacctttgccccttgcgtgcccctggccgatgggagggcggtggaggctctgtaccctggccctgctggagcatctcccccaagcccactccaggccctgggaatggccagagtctaggagaggtagaaacgatcctatcagcttctctcccacccaattaggcccagagagacaaagacagatctgaaagcaaatgcaacagagaagagacacttcttagagtaaaatgtgtctcatctctatcagccatcgcctttcatcttcccaggggcctcagaagaaggaattaagttaggctgaacaggcctcagagttaggccctggctgcttgattggctgagggggaaagagttcccttttctcattcagaaaccaaggtgctgtgtctagtcagggagccttggagatgcctggactagttggaggaatcgttggcagaggatcagagaccagcagcaggctgtctgccctgtctagagctcttcccctcaacttgtctgggcccatctgggggttgccacacaacacctaacttaccttttcctgaaagaagttgggaaaccatcatcactagaggcctttgctcagagaggagctgccttaggagtcttgggtcggaggacggggctaggaattgaccagggctttgcctgccgccctcagcagtgtcgggtacattctgacctcgcctgcagctgggctgtggattcttcctgacattcagatgtgagctgttttgggagtcagctagtatggagtacgagatgcaacccagcccccaaacctacattctgcactcaaattccaaaacactgctttactgtaaagaagaggcccctggcacccaatctccctgtccttcactgtcccctcagacctgggcggggaggggggggggcctgtgaccacctgagacatacgctcgtgacactgccccaccccagccacctccacttgcttcctcctccttccctccgctgctctttccccacggcccagaatttagctgctctgacagccacttttgagaccagctggctttgtagtcacttcagagagctggagcggctgcccactgggccctgactgggagtcccctgccagctcctgatcaggcgctgcgccctggtggcagtgatgactgggagtcccctgccagctcctgtccaggcgctgcatcctggtaacagtgaggccatgttgctgtcatctccacctctgcattcttgctgcctgtgggtcctttttctttcatggagcctgctgggtcttgtctcacctgtgctgagctcctctggggttttgatttcttccttccttatcaggccctttggggtaagcctgctggttgtacctgacatagggaggcagttaggggcagtccctggtggggccgccctggcagcctccagctggcaccatcgtgtgcctggtttccctgcaacacctgcctctctgtccctgctgctgcttggctcaggcccaacaggcagcgtgcatggaggtggttacacacagctgtttccgtgagggtgaccgtgtctgcagcacgcttccgtctccgcatgcacggctgcctctccagccacctctgatacttctctcttggggccatcagagcctcccttgggctgtcacctcccagctcacacacactcttcagtggtttcctctcttcattctcttatagggcgtggtccttcttatttatctaaagggctgaatttaggagactttttacccaggggcaaaaggctcttagggtaatgagatggatggtggcccaggtgcattttccagggcctgggttctccagatcccgtggcttctgttgagtggaggcaactttgctctgtgtgaacctcgcccctgtccctctgccgggcacccctggcaggaagcaggactcccatcctcaccctgacttagactgtcctctgagtcagctcctctccaagacaggagtgggcagccctgggcagtcttctggccccttgctaaagtgaggggcaggaagctggggctgccctccagaaagccggggtaggaactctgaaaaatacctcctctaaacggaagcagggctctccagttccacttggcgccccctcccacaaggcccttcctccctgaggaccccacccccctaccccttccccagcagcctttggaccctcacctctctccggtgtccgtgggtcctcagcccagggtgagctgcagtcaggcgggatgggacgggcaggccagaggtcagccagctcctagcagagaagagccagccagaccccaaccctgtctcttgtccatgccctttgtgatttcagtcttggtagacttgtatttggagttttgtgcttcaaagtttttgtttttgtttgtttggtttttgttttgagggggtggggggggatacagagcagctgatcaatttgtatttatttattttaacattttactaaataaagccaaataaagcctctcaaaaaaaaaaaaaaaaa

SEQ ID NO: 20 - RNA, 14135 nt, Homo sapiens
  source 1..14135: mol_type = mRNA
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacgatgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagagcctcgacacccccccaacccccgcaccaagcagacaaagtcaataaaagagctgtctgactgc

SEQ ID NO: 21 - RNA, 2207 nt, Homo sapiens
  source 1..2207: mol_type = mRNA
gcatcctctcaccgccggaagctgaactgactcgtccgcggccgctctaccccaacaggccgccaccagcgagagtgcggccataaccatcacgtgaccgcccaccgacaccagcgagagtgcagtcgtaaccgtcacgtgaccgcccaccgtcggcccggcgctcccctccgcccgaagctagcaagcggcgcggccaatgagaaaggcgcatgcctggcccccgccggcctgcagtctagccgtagtgcgcctgcgcgcggctaggaggggccgtcaggcggggatacagcctggaaggtaatgcatgtccatggtacacaaattcacaagtttggagaccctgacacacccaccttctcacctgggctctgcgtatcccccagccttgagggaagatgaagcctaaactgatgtaccaggagctgaaggtgcctgcagaggagcccgccaatgagctgcccatgaatgagattgaggcgtggaaggctgcggaaaagaaagcccgctgggtcctgctggtcctcattctggcggttgtgggcttcggagccctgatgactcagctgtttctatgggaatacggcgacttgcatctctttgggcccaaccagcgcccagccccctgctatgacccttgcgaagcagtgctggtggaaagcattcctgagggcctggacttccccaatgcctccacggggaacccttccaccagccaggcctggctgggcctgctcgccggtgcgcacagcagcctggacatcgcctccttctactggaccctcaccaacaatgacacccacacgcaggagccctctgcccagcagggtgaggaggtcctccggcagctgcagaccctggcaccaaagggcgtgaacgtccgcatcgctgtgagcaagcccagcgggccccagccacaggcggacctgcaggctctgctgcagagcggtgcccaggtccgcatggtggacatgcagaagctgacccatggcgtcctgcataccaagttctgggtggtggaccagacccacttctacctgggcagtgccaacatggactggcgttcactgacccaggtcaaggagctgggcgtggtcatgtacaactgcagctgcctggctcgagacctgaccaagatctttgaggcctactggttcctgggccaggcaggcagctccatcccatcaacttggccccggttctatgacacccgctacaaccaagagacaccaatggagatctgcctcaatggaacccctgctctggcctacctggcgagtgcgcccccacccctgtgtccaagtggccgcactccagacctgaaggctctactcaacgtggtggacaatgcccggagtttcatctacgtcgctgtcatgaactacctgcccactctggagttctcccaccctcacaggttctggcctgccattgacgatgggctgcggcgggccacctacgagcgtggcgtcaaggtgcgcctgctcatcagctgctggggacactcggagccatccatgcgggccttcctgctctctctggctgccctgcgtgacaaccatacccactctgacatccaggtgaaactctttgtggtccccgcggatgaggcccaggctcgaatcccatatgcccgtgtcaaccacaacaagtacatggtgactgaacgcgccacctacatcggaacctccaactggtctggcaactacttcacggagacggcgggcacctcgctgctggtgacgcagaatgggaggggcggcctgcggagccagctggaggccattttcctgagggactgggactccccttacagccatgaccttgacacctcagctgacagcgtgggcaacgcctgccgcctgctctgaggcccgatccagtgggcaggccaaggcctgctgggcccccgcggacccaggtgctctgggtcacggtccctgtccccgcgcccccgcttctgtctgccccattgtggctcctcaggctctctcccctgctctcccacctctacctccacccccaccggcctgacgctgtggccccgggacccagcagagctgggggagggatcagcccccaaagaaatgggggtgcatgctgggcctggccccctggcccacccccactttccagggcaaaaagggcccagggttataataagtaaataacttgtctgtacagcctggaaaaaaaaaaaaaaaaaa

SEQ ID NO: 22 - RNA, 4846 nt, Homo sapiens
  source 1..4846: mol_type = mRNA
gagcggtgctcaggggagggctggaggggagggaaggagagagagaggggagggcggcaccgcccctagccccgcgctccggaagtgaagcggccagaccaccagctaatggatgcggagcggagggcccgctgaccgctctccgcgcctggagcagcttggcttggctggagctaagagccagacacaccactgtgtggaggtgggtgatgtcttcctgtgctaaaaggtgaataaataagctcctcacctctcgcggaacactcgggaacacatcaacaggggtccaagccgccctgctgggaggcttctcttcaagagttctgggtcccagagtggaaggcattttcccatcaactggagagagacgaaacatcagagaccaggaggctgtggagaaagcagctgtcccaggtgcctcaactatcagagaagggtcagcgtcacgtggctgccagcatctttgagaaaatcactggcaatcggacttcagagctgcgggcacaggtgtggttagaactgagatacgacctgcccacctgggtcaggcctaaagacaagaagtcctgagttcttgccactgagtaggccagggtcatttgtccagaaaactttgtgactgtctttgagtgacctagtctgggacccattcattggtgggttctaaggttagaagctcatccaggatattttcaatattaagtcagtgcatagctgcaccactaacaaattggtgcctgtagagtcagagtgggtcaattcttaggacaatggcgctggcactgttagaggactggtgcaggataatgagtgtggatgagcagaagtcactgatggttacggggataccggcggactttgaggaggctgagattcaggaggtccttcaggagactttaaagtctctgggcaggtatagactgcttggcaagatattccggaagcaggagaatgccaatgctgtcttactagagcttctggaagatactgatgtctcggccattcccagtgaggtccagggaaaggggggtgtctggaaggtgatctttaagacccctaatcaggacactgagtttcttgaaagattgaacctgtttctagaaaaagaggggcagacggtctcgggtatgtttcgagccctggggcaggagggcgtgtctccagccacagtgccctgcatctcaccagaattactggcccatttgttgggacaggcaatggcacatgcgcctcagcccctgctacccatgagataccggaaactgcgagtattctcagggagtgctgtcccagccccagaggaagagtcctttgaggtctggttggaacaggccacggagatagtcaaagagtggccagtaacagaggcagaaaagaaaaggtggctggcggaaagcctgcggggccctgccctggacctcatgcacatagtgcaggcagacaacccgtccatcagtgtagaagagtgtttggaggcctttaagcaagtgtttgggagcctagagagccgcaggacagcccaggtgaggtatctgaagacctatcaggaggaaggagagaaggtctcagcctatgtgttacggctagaaaccctgctccggagagcggtggagaaacgcgccatccctcggcgtattgcggaccaggtccgcctggagcaggtcatggctggggccactcttaaccagatgctgtggtgccggcttagggagctgaaggatcagggcccgccccccagcttccttgagctaatgaaggtaatacgggaagaagaggaggaagaggcctcctttgagaatgagagtatcgaagagccagaggaacgagatggctatggccgctggaatcatgagggagacgactgaaaaccacctgggggcaggacccacagccagtgggctaagacctttaaaaaatttttttctttaatgtatgggactgaaatcaaaccatgaaagccaattattgaccttccttccttccttccttccctcccttcctccttctctccttctctcctcctctctcctctcctctcctctctttccttccttccttccttttttctttttctctttcttctttatttcttgggtctcactctcatcacccaggctagagtgcagtggcacaaaaatctcggctcactgcagccttgacttcccaggctcaggctcaggtgatcctcacaccttagcctcccaagtacctgggactacaggcacgcaccaccatgcctagctattcttttgtatttttggtagagacagggttttgctgtgttgctcaggctggtctggaacccctaggctcaaatgatgtgcccaactcggcctcccaaagtgctgggattacaggcatgaaccgccatgcctggcccttgatttttctttttaagaaaaaaatatctaggagtttcttagaccctatgtagattattaatgaacaaaagattaaactccaaatattaaatagtaagcctgaaggaatctgaaacacttgtacttccaattttctttaaataatcccaaatagaccagaattggcccataccatagaagaaagaattggcagtcaaaaaaaaaaataccttttgtaatgtttgaaaaataaagctgtttgacttgtcaggtgttttcctttctcaaatcagcaaattctctctgagtgcctggctttgtgagacactgtacaaggagttacaagactacagctataacctgcagttgagcagttataaacctacaaaatgggccctgccctcagagaggttccagtctagatgaggagctgatctagacaggtaaaaggctaactaaccctttgtgtaaataagttcatcaccccagtaaaagtgtcatcacccagtgaataggaccacctctgcctgcagatttttgttgttgttgttgtcattgttgttgttgttttaacctgggaagtgttcttcctgcctttctgctaggtgtcagatagatggtcccagagctaggtgctgtgtcaggccctgaagacacagatgactcaacctaagctttactttccagaggtccacagcctgagaggtgtccccaaagaaagggggacatgaggggactgcatgcttgagagcagggttgtttagggcaggtttggatttagtgagcaggctggtttgcttagagaaggcttttagtggcaacaaaggatgaagaggagagaaaaggaactcacatttattgagggcctactgtgtgcaaagtgtttcatgtatatctcattgaatgtatacagccaccctgttgtggtataattttgctctttataaagagaaagaccgaagctcagatgagttaagtggtctcctcaacaccaaaatgccaagaagtgatggagcctagacagaagcccagaactttctgactcacactagtccatcctctaccatcacgatgactttcaaattgtgctctgcagttctgcagattttctagcagtgccatctccaaaatgtgttttaaactctttatttttttaattattattagtattattttgagactgagtcttgctctatcacccaggctggagtgcagtggtgcaatctcagctcactgcaacctccgcctcccaggttcaagcgatttcgtgcctcagcctcccgagtagctgggattacaggcacccaccaccacgcccagctaatttttgtatttttagtagaaatggggtttcaccatgttggccaggctggtctcgaactcctgacctcaagtgatccactcacctcggcctcccaaagtgctgggattacaggtgtgagccaccatgcctgggctaaactctttaagtctctagtaaatgcagctagattcaaatgggctgataaccaaattttaacacatcagcattcaccaccaggtttacttttattttcagattggctcattttgtgcagaccttagagcaaagtttcctttatggtatctgtgtacgtatccaaacttcttttaattgttcacagattttaaaagcggtagcaccacatggttgtgtagatcagacctgtgtatttagatcagacctgtgtatcacgtaagtgtgtgagtgcagtgcagatgagcaccatttagttatatgtgctaggcaaatctccaacacagttgatgtgtagtcttgtggtagatttgtgcatactgtaagcaaattgcttagcttctctagacatcagtttccacatctgaaaaataagaagatgagagtacacggttgttatgaacaaatgacttaatgctttttaagcacgttgcatgacatctggaacacagaaagccctcaatacattgaagctcttaggattttcacgatgttcctgtctgctcaatgcatgctttctttattgttctgacagttgtgtggtaacaagctaatatgcttccagttgacttccagtctaccctggtgttagaaaccgtttcatctcttattgtaaatttgagtgcttgttgttttttatatttgtgatgactcttccagcagttgttgacaattgttagaggtttgacttttaaataattacttattttttctgattgtggttcagtttaactgaagaatatcctgagattgtaagaaaagcattttttaaaaggtatcacttgtgatcatttatctttctaaattctatttttaatactgttccaccaaagtgatgcagtggttaccatgacaccctaatttcatgtgtttttgtatttatgaaaatagtttcattgtcatttattggcggtatacaaagtaaaatgttataaatgtgaagttataaaataaatatatgctaataaaatcctgagtttttctgtttcct

SEQ ID NO: 23 - RNA, 1014 nt, Homo sapiens
  source 1..1014: mol_type = mRNA
tgcctcctgagcgtagtccagttactttcaggctcggggagtgaaggcctcgttgagagaaggtctcattcggtgttttgggaagagagtcgtgtgggcccaggtctgtctgctatcagctatgccgctgcccgttgcgctgcagacccgcttggccaagagaggcatcctcaaacatctggagcctgaaccagaggaagagatcattgccgaggactatgacgatgatcctgtggactacgaggccaccaggttggagggcctaccaccaagctggtacaaggtgttcgacccttcctgcgggctcccttactactggaatgcagacacagaccttgtatcctggctctccccacatgaccccaactccgtggttaccaaatcggccaagaagctcagaagcagtaatgcagatgctgaagaaaagttggaccggagccatgacaagtcggacaggggccatgacaagtcggaccgcagccatgagaaactagacaggggccacgacaagtcagaccggggccacgacaagtctgacagggatcgagagcgtggctatgacaaggtagacagagagagagagcgagacagggaacgggatcgggaccgcgggtatgacaaggcagaccgggaagagggcaaagaacggcgccaccatcgccgggaggagctggctccctatcccaagagcaagaaggcagtaagccgaaaggatgaagagttagaccccatggaccctagctcatactcagacgccccccggggcacgtggtcaacaggactccccaagcggaatgaggccaagactggcgctgacaccacagcagctgggcccctcttccagcagcggccgtatccatccccaggggctgtgctccgggccaatgcagaggcctcccgaaccaagcagcaggattgaagcttcggcctccctggccctgggttaaaataaaagctttctggtgatcctgcccaccaaaaaaaaaaaaaaaaaaaaaaaaaaaaaaaaaaaaa

SEQ ID NO: 24 - RNA, 3291 nt, Homo sapiens
  source 1..3291: mol_type = mRNA
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 25 - RNA, 5216 nt, Homo sapiens
  source 1..5216: mol_type = mRNA
gatgtcccaggggtattggggcggggggttgaaataactggggttcaggaggagggatggtggtagagataaaaatgtgagaagggagcagcactggcgaggagtcgggagagtactcctgattgtgacatcacattcatcccctgggcgatggagcttgtcactgggaaggaatactcagtcggagaatagccaacaagatgggttactgggagaatctcttcagtggcactgagtggaggcatcagggggttggagccttgtgaacagggaacctgccccccaacacttggaaggacctgggtttcagtgatgagacatggggtatgatgtaacccgtttccagggggatgttgacgaagatcttatctgccctatttgcagtggagtcttggaggagccagtacaggcacctcattgtgaacatgctttctgcaacgcctgcatcacccagtggttctctcagcaacagacatgtccagtggaccgtagtgttgtgacggtcgcccatctgcgcccagtacctcggatcatgcggaacatgttgtcaaagctgcagattgcctgtgacaacgctgtgttcggctgtagtgccgttgtccggcttgacaacctcatgtctcacctcagcgactgtgagcacaacccgaagcggcctgtgacctgtgaacagggctgtggcctggagatgcccaaagatgagctgcccaaccataactgcattaagcacctgcgctcagtggtacagcagcagcagacacgcatcgcagagctggagaagacgtcagctgaacacaaacaccagctggcggagcagaagcgagacatccagctgctaaaggcatacatgcgtgcaatccgcagtgtcaaccccaaccttcagaacctggaggagacaattgaatacaacgagatcctagagtgggtgaactcccttcagccagcaagagtgacccgctggggagggatgatctcgactcctgatgctgtgctccaggctgtaatcaagcgctccctggtggagagtggctgtcctgcttctattgtcaacgagctgattgaaaatgcccacgagcgtagctggccccagggtctggccacactagagactagacagatgaaccgacgctactatgagaactacgtggccaagcgcatccctggcaagcaggctgttgtcgtgatggcctgtgagaaccagcacatgggggatgacatggtgcaagagccaggccttgtcatgatatttgcgcatggcgtggaagagatataagagaactcgactggctatcaggaagagatggaaatcagaaaatcccatcactccagcagctgggacctgagtcctacccaccattcttaatactgtggcttatacctgagccacacatctccctgcccttctggcactgaagggccttggggtagtttgctcagcctttcaggtgggaaacccagatttcctccctttgccatattcccctaaaatgtctataaattatcagtctgggtgggaaagcccccacctccatccattttcctgcttagggtccctggttccagttattttcagaaagcacaaagagattcaatttccctggaggatcaggacagaggaaggaatctctaatcgtccctctcctccaaaaccagggaatcagagcagtcaggcctgttgactctaagcagcagacatcctgaagaaatggtaagggtggagccaaatctctagaaataagtagtgaggccgttaattggccatcactgatggcccttagggaaagactggacctctgtgccaagcagtatccctgttcagcccaccttaaaggtgtaggcacccactgggtctaccagtatgcaggttgggatactgaaaatttccagatgagctcttctttcctacaagttttcataattagggaatgccagggtttagggtaggggttaatctgttgggggttgatgtgtttagcaagaagctactcctagcttttgctaaaatatggttggcactgcctcttgtggcacaggccataattgttccatagacccctctctagccctgtgactgtagttagttactttgataattttctttggccattgtttgtttatatttcacaaactccacctactgcccccccccctcttttttttaagaatggcctgatcatggctatctcagccacattgttggcaatttaatttatttacttcctttttttttttttaagaaaggaaaaaagaaaaaaaaatcaaacttgaaacttttcttttgatgttcctattgtgggggttctggatagggtgggacagggatgggggtgtgttttatattttttccttttcagcacaacctttggctttaatataggaagagccaagggagtcctcggctgaacttacgatatctgccccaaacctctgtaaccccaactgaaatgaggagcttcctctcttcctgtgaaggatatgacagtccagcatcgatgcctgtgccctctggaaaaatttcctcctagcccttccagggccttatcataaaactctggatttagagtattcattttgaaggcaactcccccttccccaagtttccttggagctgtatagctgggttctaagcttcaccatgcaaatcagaaattttatctctaagtacaggctgtgccgtgtctcacccacacccccctggggacttcagttccatttcaggttacctggggtataccttgatccctagagtgactggcagagtaagagaaggggagagataataggtgtgattattttaatatggaggtgggagtgtggttggagatagaaaggctcctccccaccatgtaatggcttcctctcagaattttattccaggctagcttgctgcaggtctgggtagttggatcatggctccactgggattggggtggaaagcttgaggggagtagggttccagctctgggacattgtgctcaggaatttgaaaacgctgctatacttactctggttactacatttcttccactcccctttcccctacctgccttaaccaaggctcatactgtcctgtccttaccctcagatggagccaggaagctcagtgaaaggcttccctaccctttgcactagtgtctctgcaggttgctggttgtgttgtatgtgctgttccatggtgttgactgcactaataataaaccttttactcaactctctaaattcttcagcattactccctttcttgagaaggtttcccctctgcttttgcctttctctcaccttaattccctttcttccttactttgttacctacccttatcttagtgctaacttctctttcaggaggatgtctgggagtagtgtgcacttcacagctgctttcccatgtaccctcctgcattcttccctcctatctcctgttctgtagcagccaaagctctctagtgatctgaactgtgtgcttcccagggtctgcctttatcctaaattccatgtcttccctgagtggtcctgagtttttgggataatttctacagaagatatgtatatatctttttcctttgtcccacaagcaactttgctttagaatctagaattcctttgcaggcagagaagtctctacctcccagtgtttcctagctaagaacgtaaatgtgaggagggaaatgtacttgcagaggtttcataattatttacttataaaaatagtcttcatagccgggcgcggtggctcacgcctgtaatcccagcactttgggaggccgaggtgggtggatcacaaggtcaggagttcgagaccatcctggctaacacagtgaaaccccgtctctactaaaaatacaaaaaattagccgggcgtggtggcaggcacctgtagtcccagctacttaggaggctgaggcaggagaatggcgtgaacccgggaggcagagcttgcagtgagcagagattgggccactgcattccagcctgggcgacagagcaaggctccgtctaaaaaaaaaaaaaaaaaaaaaagtcttcataggccgggcacggtggctcacgtctgtaatcccagcactttgggaggccaaggtgggtggatcacaacgtcaggagatcgagaccatcctggctaacatggtgaaaccctgtctctactaaaaatataaataaattagccggacaggcgcctgtcctcccagctactcaggaggctgaggcaggagaatggtgtgaacctgggaggcggagcttgcagtgagctgagatcacgccactgcactccagcctgggcaacagagcaagactccgtctcaaaaaaaaaaaaaaaaaaaccagtcttcataagtatttgctgctacctttccctgtcataagaaaaaggatagccagacatggtgggacgccactatgatcccagctccttggaaggctaaggcacaagaatcgcttgaacctgggaggtggaggttgcagtgagctgagatcatgccactgcactccagcctggtgacagagcaagagcctgtctcaaaaaaaaaaaagaaaagaaaagaaaaagggatatcttttcctcctcccagaagtttgttttaaatttgagcatttatcatgcacctgatgtaaacctaatagtactcttgatactctagtggcttgaaaaaaaaaaaaaaggcatttctgtgctgagtctgcgcttctatgcacacaaggtatgtttataaaatactgataagcatgtcacagtatagagcataagaggcaatgtatgtatcctagtgacattagcagtgcttttccccccttaaactcctttaaaattacttttagaacttgctgctcattcttgtgaatgttatgaatggtgtcatattgtccttttacagaagatacgatttttagaaacaaatattcattgaatgtctgccctgtgagatactcactagagtgaacatgaggaggcttatgtagcaaaatggcacctacctgcaaagaacttagtccctaatggagatgaatatataataagggatcataaatgtgctaagtggatttactagtaatatgtgagccaaggacgataaagctcctgattctgatgggtatcaggaaaggcttttcaggaagtgttacttgttataggtcagaggtcagcaaactacaggttacaaccccactgcctgcttttgtaaaaaactttattggaatacagttatgcccacttgtttata

SEQ ID NO: 26 - RNA, 5138 nt, Homo sapiens
  source 1..5138: mol_type = mRNA
gatccgcagaggagcccacttgagagcgcctcctgtcgtctgtaaggttgccttgccatccctcggcaccccaacttcccccgcccccccatcgcctcctcctccatcctccagttcaaaatggcgacggcggcggcagcggcggcggtgatggctcctccgggctgcccgggttcgtgccccaacttcgccgtagtctgctccttcttggagcgctacgggccgctgctagacctgcctgagttgccgttccctgagctggagcgggtgctgcaggcgccgccgccggacgtcggcaacggagaagtaccaaaagaattggtggagctccatttgaagctgatgaggaaaattggcaaatctgttactgcagacagatgggaaaaatatttgatcaagatatgccaagagtttaacagtacctgggcatgggagatggagaagaagggctatcttgaaatgagtgttgaatgcaaactagcactcttaaagtacctctgtgagtgtcagtttgatgacaatctcaaattcaagaatattattaatgaggaggatgccgatactatgcgtctccagccaattggtcgagacaaagatggcctcatgtactggtaccaattggatcaagatcacaatgtcagaatgtacatagaagaacaagatgatcaagatggctcttcatggaaatgcattgtcagaaatcgaaacgagttggctgagactcttgcactcctgaaagcacaaattgatcctgtactattgaaaaactctagccaacaagacaactcttctcgggaaagtcccagcttagaggatgaggagactaaaaaagaggaagaaacacctaaacaagaggaacagaaagaaagtgaaaagatgaaaagtgaggagcagcctatggatttagaaaaccgttctacagccaatgttctagaagagactactgtgaaaaaagaaaaagaagatgaaaaggaacttgtgaaactgccagtcatagtgaagctagaaaaacctttgccagaaaatgaagaaaaaaagattatcaaagaagaaagtgattccttcaaggaaaatgtcaaacccattaaagttgaggtgaaggaatgtagagcagatcctaaagataccaaaagtagcatggagaagccagtggcacaggagcctgaaaggatcgaatttggtggcaatattaaatcttctcacgaaattactgagaaatctactgaagaaactgagaaacttaaaaatgaccagcaggccaagataccactaaaaaaacgagaaattaaactgagtgatgattttgacagtccagtcaagggacctttgtgtaaatcagttactccaacaaaagagtttttgaaagatgaaataaaacaagaggaagagacttgtaaaaggatctctacaatcactgctttgggtcatgaagggaaacagctggtaaatggagaagttagtgatgaaagggtagctccaaattttaagacagaaccaatagagacaaagttttatgagacaaaggaagagagctatagcccctctaaggacagaaatatcatcacggagggaaatggaacagagtccttaaattctgtcataacaagtatgaaaacaggtgagcttgagaaagaaacagcccctttgaggaaagatgcagatagttcaatatcagtcttagagatccatagtcaaaaagcacaaatagaggaacccgatcctccagaaatggaaacttctcttgattcttctgagatggcaaaagatctctcttcaaaaactgctttatcttccaccgagtcgtgtaccatgaaaggtgaagagaagtctcccaaaactaagaaggataagcgcccaccaatcctagaatgtcttgaaaagttagagaagtccaaaaagacttttcttgataaggacgcacaaagattgagtccaataccagaagaagttccaaagagtactctagagtcagaaaagcctggctctcctgaggcagctgaaacttctccaccatctaatatcattgaccactgtgagaaactagcctcagaaaaagaagtggtagaatgccagagtacaagtactgttggtggccagtctgtgaaaaaagtagacctagaaaccctaaaagaggattctgagttcacaaaggtagaaatggataatctggacaatgcccagacctctggcatagaggagccttctgagacaaagggttctatgcaaaaaagcaaattcaaatataagttggttcctgaagaagaaaccactgcctcagaaaatacagagataacctctgaaaggcagaaagagggcatcaaattaacaatcaggatatcaagtcggaaaaagaagcccgattctccccccaaagttctagaaccagaaaacaagcaagagaagacagaaaaggaagaggagaaaacaaatgtgggtcgtactttaagaagatctccaagaatatctagacccactgcaaaagtggctgagatcagagatcagaaagctgataaaaaaagaggggaaggagaagatgaggtggaagaagagtcaacagctttgcaaaaaactgacaaaaaggaaattttgaaaaaatcagagaaagatacaaattctaaagtaagcaaggtaaaacccaaaggcaaagttcgatggactggttctcggacacgtggcagatggaaatattccagcaatgatgaaagtgaagggtctggcagtgaaaaatcatctgcagcttcagaagaggaggaagaaaaggaaagtgaagaagccatcctagcagatgatgatgaaccatgcaaaaaatgtggccttccaaaccatcctgagctaattcttctgtgtgactcttgcgatagtggataccatactgcctgccttcgccctcctctgatgatcatcccagatggagaatggttctgcccaccttgccaacataaactgctctgtgaaaaattagaggaacagttgcaggatttggatgttgccttaaagaagaaagagcgtgccgaacgaagaaaagaacgcttggtgtatgttggtatcagtattgaaaacatcattcctccacaagagccagacttttctgaagatcaagaagaaaagaaaaaagattcaaaaaaatccaaagcaaacttgcttgaaaggaggtcaacaagaacaaggaaatgtataagctacagatttgatgagtttgatgaagcaattgatgaagctattgaagatgacatcaaagaagccgatggaggaggagttggccgaggaaaagatatctccaccatcacaggtcatcgtgggaaagacatctctactattttggatgaagaaagaaaagaaaataaacgaccccagagggcagctgctgctcgaaggaagaaacgccggcgattaaatgatctggacagtgatagcaacctggatgaagaagagagcgaggatgaattcaagatcagtgatggatctcaagatgagtttgttgtgtctgatgaaaacccagatgaaagtgaagaagatccgccatctaatgatgacagtgacactgacttttgtagccgtagactgaggcgacacccctctcggccaatgaggcagagcaggcgtttgcgaagaaagaccccaaagaaaaaatattccgatgatgatgaagaggaggaatctgaggagaatagtagagactctgaaagtgacttcagtgatgattttagtgatgattttgtagaaactcggcgaaggcggtcaaggagaaatcagaaaagacaaattaactacaaagaagactcagaaagtgacggttcccagaagagtttgcgacgtggtaaagaaataaggcgagtacacaagcgaagactttccagctcagagagtgaagagagctatttgtccaagaactctgaagatgatgagctagctaaagaatcaaagcggtcagttcgaaagcggggccgaagcacagacgagtattcagaagcagatgaggaggaggaggaagaggaaggcaaaccatcccgcaaacggctacaccggattgagacggatgaggaggagagttgtgacaatgctcatggagatgcaaatcagcctgcccgtgacagccagcctagggtcctgccctcagaacaagagagcaccaagaagccctaccggatagaaagtgatgaggaagaggactttgaaaatgtaggcaaagtggggagcccattggactatagcttagtggacttaccttcaaccaatggacagagccctggcaaagccattgagaacttgattggcaagcctactgagaagtctcagacccccaaggacaacagcacagccagtgcaagcctagcctccaatgggacaagtggtgggcaggaggcaggagcaccagaagaggaggaagatgagcttttgagagtgactgaccttgttgattatgtctgtaacagtgaacagttataagactttttttccatttttgtgctaatttattccacggtagctctcacaccagcgggccagttattaaaagctgtttaatttttcctagaaaactccactacagaatgacttttagaagaaaaatttcaacaaatcctgaagtctttctgtgaagtgaccagttctgaactttgaagataaataattgctgtaaattccttttgattttctttttccaggttcatggtccttggtaatttcattcatggaaaaaaatcttattataataacaacaaagatttgtatatttttgactttatatttcctgagctctcctgactttgtgaaaaagggtggatgaaaatgcattccgaatctgtgagggcccaaaacagaatttaggggtgggtgaaagcacttgtgctttagctttttcatattaaatatatattatatttaaacattcatggcatagatgatgatttacagacaatttaaaagttcaagtctgtactgttacagtttgagaattgtagataacatcatacataagtcatttagtaacagcctttgtgaaatgaacttgtttactattggagataaccacacttaataaagaagagacagtgaaagtaccatcataattaacctaaatttttgttatagcagagtttcttgtttaaaaaaaaataaaatcatctgaaaagcaaaaa

SEQ ID NO: 27 - RNA, 2288 nt, Homo sapiens
  source 1..2288: mol_type = mRNA
cgcgcgctgcagtgccttccccacctcggccccgcccgcccccgccgagccgagcaccagggcggcggcggcggcggcggcggcggcggcggctggagcagcccgggaggaggaggcggcgagaatggcagcggcgtcgtgggcgcggcggagatgagcgcccgcgaccccgggcccagggcggcacagccggagtgggcgggggtcccgatgcaggcccgaggggggccatggggcaggtcctgccggtcttcgcccactgcaaagaagctccgtctacagcctcctcaactcctgattccacagaaggagggaacgacgactctgattttcgagagctgcacacagcccgggaattctcagaggaggacgaggaggagaccacgtcgcaggactggggcaccccccgggagctgaccttctcctacatcgcctttgatggtgtagtgggctccgggggccgcagggattcaactgcccgccgcccccgcccccagggccgctcagtctcggaaccacgagaccagcaccctcagcccagcctgggcgacagcttggagagcatccccagcctgagccaatccccggagcctggacgacggggtgatcctgacaccgcgcctccatccgagcgccctctggaagacctgaggcttcggttggaccatctgggctgggtggcccggggaacgggatccggggaggactcttccaccagcagctccaccccgctggaagacgaagaaccccaagaacccaacagattggagacaggagaagctggggaagaactggacctacgactccgacttgctcagccctcatcgcccgaggtcttgactccccagctcagtccgggctctgggacaccccaggccggtactccgtccccatcccgatcgcgagattcgaactctgggcccgaagagccattgctggaagaggaagaaaagcagtgggggccactggagcgagagccagtaaggggacagtgcctcgatagcacggaccaattagaattcacggtggagccacgccttctaggaacagctatggaatggttaaagacatcattgcttttggctgtttacaagacggttccaattttggaattgtccccacctctgtggacagccattggctgggtccaaaggggccccaccccccctactcctgtcctccgggttctactgaagtgggcaaaatccccgagaagcagcggtgtccccagcctctcactcggagccgatatggggagtaaagtggcggacctgctgtactggaaggacacgaggacgtcaggagtggtcttcacaggcctgatggtctccctcctctgcctcctgcactttagcatcgtgtccgtggccgcgcacttggctctgttgctgctctgcggcaccatctctctcagggtttaccgcaaagtgctgcaggccgtgcaccggggggatggagccaaccctttccaggcctacctggatgtggacctcaccctgactcgggagcagacggaacgtttgtcccaccagatcacctcccgcgtggtctcggcggccacgcagctgcggcacttcttcctggtagaagacctcgtggattccctcaagctggccctcctcttctacatcttgaccttcgtgggtgccatcttcaatggtttgactcttctcattctgggagtgattggtctattcaccatccccctgctgtaccggcagcaccaggctcagatcgaccaatatgtggggttggtgaccaatcagttgagccacatcaaagctaagatccgagctaaaatcccagggaccggagccctggcctctgcagcagccgcagtctccggatccaaagccaaagccgaatgagaacggtgtctctgcccgcaggacgcctgcccccagcccccgcagccctctggccccctccatctcttgtccgttcccacccacccccctcctcggcccgagccttttcccggtgggtgtcaggatcactcccactagggactctgcgctaattacctgagcgaccaggactacatttcccaagaggctctgctccaggagtccaggaaagacgaggcaccttggccgcggggcctgctgggacttgtagttgcctagacagggcaccaccctgcacttccggacccgccgctggaggcgccgtgaggcgttggtgtctcctggatgctactagccccaacgccggggctttgcatggggcccaggggaggcctgagcttggatttacactgtaataaagactcctgtggaaaacccgag

SEQ ID NO: 28 - RNA, 1700 nt, Homo sapiens
  source 1..1700: mol_type = mRNA
agcaggactcagaggggagagttggaggaaaaaaaaaggcagaaaagggaaagaaagaggaagagagagagagagtgagaggagccgctgagcccaccccgatggccgcggacgaagttgccggaggggcgcgcaaagccacgaaaagcaaactttttgagtttctggtccatggggtgcgccccgggatgccgtctggagcccggatgccccaccagggggcgcccatgggccccccgggctccccgtacatgggcagccccgccgtgcgacccggcctggcccccgcgggcatggagcccgcccgcaagcgagcagcgcccccgcccgggcagagccaggcacagagccagggccagccggtgcccaccgcccccgcgcggagccgcagtgccaagaggaggaagatggctgacaaaatcctccctcaaaggattcgggagctggtccccgagtcccaggcttacatggacctcttggcatttgagaggaaactggatcaaaccatcatgcggaagcgggtggacatccaggaggctctgaagaggcccatgaagcaaaagcggaagctgcgactctatatctccaacacttttaaccctgcgaagcctgatgctgaggattccgacggcagcattgcctcctgggagctacgggtggaggggaagctcctggatgatcccagcaaacagaagcggaagttctcttctttcttcaagagtttggtcatcgagctggacaaagatctttatggccctgacaaccacctcgttgagtggcatcggacacccacgacccaggagacggacggcttccaggtgaaacggcctggggacctgagtgtgcgctgcacgctgctcctcatgctggactaccagcctccccagttcaaactggatccccgcctagcccggctgctggggctgcacacacagagccgctcagccattgtccaggccctgtggcagtatgtgaagaccaacaggctgcaggactcccatgacaaggaatacatcaatggggacaagtatttccagcagatttttgattgtccccggctgaagttttctgagattccccagcgcctcacagccctgctattgccccctgacccaattgtcatcaaccatgtcatcagcgtggacccttcagaccagaagaagacggcgtgctatgacattgacgtggaggtggaggagccattaaaggggcagatgagcagcttcctcctatccacggccaaccagcaggagatcagtgctctggacagtaagatccatgagacgattgagtccataaaccagctcaagatccagagggacttcatgctaagcttctccagagaccccaaaggctatgtccaagacctgctccgctcccagagccgggacctcaaggtgatgacagatgtagccggcaaccctgaagaggagcgccgggctgagttctaccaccagccctggtcccaggaggccgtcagtcgctacttctactgcaagatccagcagcgcaggcaggagctggagcagtcgctggttgtgcgcaacacctaggagcccaaaaataagcagcacgacggaactttcagccgtgtcccgggccccagcattttgccccgggctccagcatcactcctctgccaccttggggtgtggggctggattaaaagtcattcatctgacaaaaaaaaaaaaaaaaaa

SEQ ID NO: 29 - RNA, 4343 nt, Homo sapiens
  source 1..4343: mol_type = mRNA
tggtcatcgcacggcggcagctcctcacctggatttagaagagctggcgtccccgcccgcccaagcctttaaactctcgtctgccagaacccgccaactctccaggcttagggccagtttccgcgattctaagagtaattgcgtgggcacctgtgctggggccaggcgcaaagaagggagttggtctgcgcgaagatcgtcaacctgctaacagaccgcacatgcactttgcaccgaccatctacgtctcagtctggaggttgcgcactttggctgctgacgcgctggtggtgcctattaatcatttaccagtccagagccgcgccagttaatggctgtgccgtgcggtgctcccacatcctggcctctcctctccacggtcgcctgtgcccgggcaccccggagctgcaaactgcagagcccaggcaaccgctgggctgtgcgccccgccggcgccggtaggagccgcgctccccgcagcggttgcgctctacccggaggcgctgggcggctgtgggctgcaggcaagcggtcgggtggggagggagggcgcaggcggcgggtgcgcgaggagaaagccccagccctggcagccccactggcccccctcagctgggatgttccccaatggcaccgcctcctctccttcctcctctcctagccccagcccgggcagctgcggcgaaggcggcggcagcaggggccccggggccggcgctgcggacggcatggaggagccagggcgaaatgcgtcccagaacgggaccttgagcgagggccagggcagcgccatcctgatctctttcatctactccgtggtgtgcctggtggggctgtgtgggaactctatggtcatctacgtgatcctgcgctatgccaagatgaagacggccaccaacatctacatcctaaatctggccattgctgatgagctgctcatgctcagcgtgcccttcctagtcacctccacgttgttgcgccactggcccttcggtgcgctgctctgccgcctcgtgctcagcgtggacgcggtcaacatgttcaccagcatctactgtctgactgtgctcagcgtggaccgctacgtggccgtggtgcatcccatcaaggcggcccgctaccgccggcccaccgtggccaaggtagtaaacctgggcgtgtgggtgctatcgctgctcgtcatcctgcccatcgtggtcttctctcgcaccgcggccaacagcgacggcacggtggcttgcaacatgctcatgccagagcccgctcaacgctggctggtgggcttcgtgttgtacacatttctcatgggcttcctgctgcccgtgggggctatctgcctgtgctacgtgctcatcattgctaagatgcgcatggtggccctcaaggccggctggcagcagcgcaagcgctcggagcgcaagatcaccttaatggtgatgatggtggtgatggtgtttgtcatctgctggatgcctttctacgtggtgcagctggtcaacgtgtttgctgagcaggacgacgccacggtgagtcagctgtcggtcatcctcggctatgccaacagctgcgccaaccccatcctctatggctttctctcagacaacttcaagcgctctttccaacgcatcctatgcctcagctggatggacaacgccgcggaggagccggttgactattacgccaccgcgctcaagagccgtgcctacagtgtggaagacttccaacctgagaacctggagtccggcggcgtcttccgtaatggcacctgcacgtcccggatcacgacgctctgagcccgggccacgcaggggctctgagcccgggccacgcaggggccctgagccaaaagagggggagaatgagaagggaaggccgggtgcgaaagggacggtatccagggcgccagggtgctgtcgggataacgtggggctaggacactgacagcctttgatggaggaacccaagaaaggcgcgcgacaatggtagaagtgagagctttgcttataaactgggaaggctttcaggctacctttttctgggtctcccactttctgttccttcctccactgcgcttactcctctgaccctccttctattttccctaccctgcaacttctatcctttcttccgcaccgtcccgccagtgcagatcacgaactcattaacaactcattctgatcctcagcccctccagtcgttatttctgtttgtttaagctgagccacggataccgccacgggtttccctcggcgttagtccctagccgcgcggggccgctgtccaggttctgtctggtgcccctactggagtcccgggaatgaccgctctccctttgcgcagccctaccttaaggaaagttggacttgagaaagatctaagcagctggtcttttctcctactcttgggtgaaggtgcatctttccctgccctcccctgtccccctctcgccgcccgcccgccaccaccactctcactccacccagagtagagccaggtgcttagtaaaataggtcccgcgcttcgaactccaggctttctggagttcccacccaagccctcctttggagcaaagaaggagctgagaacaagccgaatgaggagtttttataagattgcggggtcggagtgtgggcgcgtaataggaatcaccctcctactgcgcgttttcaaagaccaagcgctgggcgctcccgggccgcgcgtctgcgttaggcagggcagggtagtgcagggcacaccttccccggggttcggggttcggggttcggttgcagggctgcagcccgccttggctttctccctcacccaagtttccggaggagccgacctaaaagtaacaatagataaggtttcctgctccagtgtatctcaaaagaccgggcgccaggggcgggggacctagggcgacgtcttcagagtccgccagtgttggcggtgtcgccgcaacctgcaggctcccgagtggggcctgcctggtctctagagggttgctgcctttcaagcggtgcctaagaagttattttcttgtttaacatatatatttattaatttatttgtcgtgttggaaaatgtgtctctgctttccttttctctgcttgcctagccccaggtcttttctttgggaccctgggggcgggcatggaagtggaagtaggggcaagctcttgccccactccctggccatctcaacgcctctcctcaatgctgggccctcttatctcatcctttcctctagcttttctatttttgattgtgttgagtgaagtttggagatttttcatacttttcttactatagtctcttgtttgtcttattaggataatacataaatgataatgtgggttatcctcctctccatgcacagtggaaagtcctgaactcctggctttccaggagacatatataggggaacatcaccctatatataatttgagtgtatatatatttatatatatgatgtggacatatgtatacttatcttgctccattgtcatgagtccatgagtctaagtatagccactgatggtgacaggtgtgagtctggctggaacactttcagtttcaggagtgcaagcagcactcaaacctggagctgaggaatctaattcagacagagactttaatcactgctgaagatgcccctgctccctctgggttccagcagaggtgattcttacatatgatccagttaacatcatcactttttttgaggacattgaaagtgaaataatttgtgtctgtgtttaatattaccaactacattggaagcctgagcagggcgaggaccaataattttaattatttatatttcctgtattgctttagtatgctggcttgtacatagtaggcactaaatacatgtttgttggttgattgtttaagccagagtgtattacaacaatctggagatactaaatctggggttctcaggttcactcattgacatgatatacaatggttaaaatcactattgaaaaatacgttttgtgtatatttgcttcaacaactttgtgctttcctgaaagcagtaaccaagagttaagatatccctaatgttttgcttaaactaatgaacaaatatgctttgggtcataaatcagaaagtttagatctgtcccttaataaaaatatatattactactcctttggaaaatagatttttaatggttaagaactgtgaaatttacaaatcaaaatcttaatcattatccttctaagaggatacaaatttagtgctcttaacttgttaccattgtaatattaactaaataaacagatgtattatgctgttaaaaaaaaaaaaaaaaaaaaaaaaaaaaaaaaaaaaaaaaaaaaaaaaaaaa

SEQ ID NO: 30 - RNA, 4171 nt, Homo sapiens
  source 1..4171: mol_type = mRNA
ctgcatctctccctctcacccgtgtctcctctcctctctttccttctcgtcttctccctgtcacgcatctctcatcactccccctcattctgcctttcctcctactcacggtctcctctccctctccctctctctctctccccctccctctttctctctctctctctttctccacctcctcccgaccccctttcccctctatttctattggcttctgtgtcccttgctcccctcttctcttcctcaccctgggaagcttctcccccctatccttgcccctgcccccccaggatgtgtcctggagatggggggtgacgtaccaggctctggttgggaagtcagggccggagaccagatgggagaggctctgtggacagccgtggccgagggcctgggagggaacctgagcccgcaagcggtctagaagtgggtgccttgtggggaccctagttaggagtgccctgggggcacctggggactgggcagggagaggggacagcagaatgataaccagcctggcggcaaggagggaagccctcaccccatgggcaggcaaatagctgactgctgaccaccctcccctcagccatggacatgcttcatccatcatcggtgtccacgacctcagaacctgagaatgcctcctcggcctggcccccagatgccaccctgggcaacgtgtcggcgggcccaagcccggcagggctggccgtcagtggcgttctgatccccctggtctacctggtggtgtgcgtggtgggcctgctgggtaactcgctggtcatctatgtggtcctgcggcacacggccagcccttcagtcaccaacgtctacatcctcaacctggcgctggccgacgagctcttcatgctggggctgcccttcctggccgcccagaacgccctgtcctactggcccttcggctccctcatgtgccgcctggtcatggcggtggatggcatcaaccagttcaccagcatattctgcctgactgtcatgagcgtggaccgctacctggccgtggtacatcccacccgctcggcccgctggcgcacagctccggtggcccgcacggtcagcgcggctgtgtgggtggcctcagccgtggtggtgctgcccgtggtggtcttctcgggagtgccccgcggcatgagcacctgccacatgcagtggcccgagccggcggcggcctggcgagccggcttcatcatctacacggccgcactgggcttcttcgggccgctgctggtcatctgcctctgctacctgctcatcgtggtgaaggtgcgctcagctgggcgccgggtgtgggcaccctcgtgccagcggcggcggcgctccgaacgcagggtcacgcgcatggtggtggccgtggtggcgctcttcgtgctctgctggatgcccttctacgtgctcaacatcgtcaacgtggtgtgcccactgcccgaggagcctgccttctttgggctctacttcctggtggtggcgctgccctatgccaacagctgtgccaaccccatcctttatggcttcctctcctaccgcttcaagcagggcttccgcagggtcctgctgcggccctcccgccgtgtgcgcagccaggagcccactgtggggcccccggagaagactgaggaggaggatgaggaggaggaggatggggaggagagcagggaggggggcaaggggaaggagatgaacggccgggtcagccagatcacgcagcctggcaccagcgggcaggagcggccgcccagcagagtggccagcaaggagcagcagctcctaccccaagaggcttccactggggagaagtccagcacgatgcgcatcagctacctgtaggggcctggggaaagccaggatggcccgaggaagaggcagaagccgtgggtgtgcctagggcctacttcccaaggtgccacaggcccatgatgggatgttgaggggcctggactttgatgctattgctgccaggtcttgctgtgtgaccttgggtaggttgcttctactctctgggccttgttttctcctctgtgactcagggataggagtcatcagcctggatgagctatgtcagatgagaggtttggagggcactgttgctgggctgacctggctgagcaggcaaaaggtgggtgcagactggcctccccccagggatggagtgtcttggggcatcaactagaatcttggccctcagagggataaaccaaggccaggatttcttgggctcagagtcaggaacacaggagctgctgggggctgggctggaaacctaaacagaagaaagcctaacccggtgggaggagtggggcagaaatggtcaggccccagatcagctccctcccctcgactgtgaggccttggaccagctctgctcctctctaggcctcaggcttcacctgggtaaaacccaacaacctctacacccttttggcccaggcagtcaatgctggaggtcctgtgctcctggacgggaagagcaggtgaatttcctgctcatggaagcgaatgaagtccagcttcagggtctctcactgcctgggcttttgcaaggccctgcatctacttttgtacttgtcattttgtattcgttttcttaaagagggacctcgaactgcataagcttaggccacccaaagcctggctctgcccctgctgaggtcagccacccaatccccaaggaagctcatgttgggtcttatggctggagtaggggcccccgggggttcccaggtcttttgagggcttccaggcacctccttgtaggaagggccatccctgttcctctccttgtgacccatattctcccttcctggagaccgagacagggacccagcccatgaggactggcatggaaaggcagagtgtctgaagagcgctgtgaggagaaggaagaggaagggagaagaggaagaggaaggagaaggaagaggaagacaagggggaaaggggaggatgaggagggggaaggagaagtacagatctgtttcctggagccgtctttggcccccctgggctgagctcagtggtagcatctgtgaacctgagttgccgacaacagccccacccaaccagtactgagggaaggacacgatcagggtggaacagccagggtgcaatggcaaatgcacagagtacagacaggcacagggcctgcgtccctgaggggcctcagagtgctgccaagagggctcaggccttaataaagccctagggtggagctggctaccagggacattgggaggactggggagctccctccccatgctctatcatcctggagactacaggtcgggaggcccagggaagacaagaagaggctgaagtgggactgtggagggggaccatggggagcagccaccatccaaggctgggcctagactccctcccagagatggtccctcagagctgtggtgaggctggccctgggagggtgagacccccggtgaaatccttccgcttccccaccccttgcagagggcaggggtcctcagggaaagcacaggaaccagacttttggagacttggatcttcagcacacctcagggtcctgggctggcattggccttccgggcctcaatttccccatcaacaaatggagatgaatcccagcttggctgcctcctgggatctaacgagaaaatgagtcatgtgaggtaacttccaggctcactgcaatgggtacggtggggtgtatcagattataaagtgggggtgccctcctcacccccaggcttggcctatacccccctctccatcaagtggcctctctgtgtctgtcctttggggtgaggacactgtaggccatgagaaatgggcagttggggggtcagaggccaagggttagggaggcagggcttggggagagtgtgggaccatcagaagagaaggaagtttacaaaaccacattttgtgtggagatggaggctggaggcccggccctgggacttggtctggggtttcttgaggaagatctgagggtccaagggaggaaggatgccctggccttctggccttctctggctgatcctgccttcttgctgcctaggacaggagagtaatgtcctagaatggtccctgggaggccagttaggaaaccctttgctgcttctgtctctagctcttgtcaataaagacggtgacacctgaaaaaaaaaaaaaaaaa

SEQ ID NO: 31 - RNA, 1258 nt, Homo sapiens
  source 1..1258: mol_type = mRNA
ccgagctctctggcgcagcgctagctccgccgcgctcagctgccctgcgccggcacccctggtcatgagcgccccctcgacgctgccccccgggggcgaggaagggctggggacggcctggccctctgcagccaatgccagtagcgctccggcggaggcggaggaggcggtggcggggcccggggacgcgcgggcggcgggcatggtcgctatccagtgcatctacgcgctggtgtgcctggtggggctggtgggcaacgccctggtcatcttcgtgatccttcgctacgccaagatgaagacggctaccaacatctacctgctcaacctggccgtagccgacgagctcttcatgctgagcgtgcccttcgtggcctcgtcggccgccctgcgccactggcccttcggctccgtgctgtgccgcgcggtgctcagcgtcgacggcctcaacatgttcaccagcgtcttctgtctcaccgtgctcagcgtggaccgctacgtggccgtggtgcaccctctgcgcgcggcgacctaccggcggcccagcgtggccaagctcatcaacctgggcgtgtggctggcatccctgttggtcactctccccatcgccatcttcgcagacaccagaccggctcgcggcggccaggccgtggcctgcaacctgcagtggccacacccggcctggtcggcagtcttcgtggtctacactttcctgctgggcttcctgctgcccgtgctggccattggcctgtgctacctgctcatcgtgggcaagatgcgcgccgtggccctgcgcgctggctggcagcagcgcaggcgctcggagaagaaaatcaccaggctggtgctgatggtcgtggtcgtctttgtgctctgctggatgcctttctacgtggtgcagctgctgaacctcttcgtgaccagccttgatgccaccgtcaaccacgtgtcccttatccttagctatgccaacagctgcgccaaccccattctctatggcttcctctccgacaacttccgccgattcttccagcgggttctctgcctgcgctgctgcctcctggaaggtgctggaggtgctgaggaggagcccctggactactatgccactgctctcaagagcaaaggtggggcagggtgcatgtgccccccactcccctgccagcaggaagccctgcaaccagaacccggccgcaagcgcatccccctcaccaggaccaccaccttctgaggagcccttcccctacccaccctgcgt

SEQ ID NO: 32 - RNA, 4427 nt, Homo sapiens
  source 1..4427: mol_type = mRNA
cccccggcggagccagctgctgctcttcggtgctggccccggtgccggccccgttgcccagggaacaggctcccggcagcccccgcggcccggagtccatcccgcctcctccggcccggcggggccgacgagtccggaggggctgccgcgggagcccccaggtttccctagatgacaaataaacattccttttcctgcgtgaagatagtctgtggaaaccttggccatggcatcgatatcagagcctgttacattcagagagttctgcccgttgtactatctcctcaatgccattccgacaaagatccagaagggtttccgctctatcgtggtctatctcacggccctcgacaccaacggggactacatcgcggtgggcagcagcatcggcatgctctatctgtactgccggcacctcaaccagatgaggaagtacaactttgaggggaagacggaatctatcactgtggtgaagctgctgagctgctttgatgacctggtggcagcaggcacagcctctggcagggttgcagtttttcaacttgtatcttcattgccagggagaaataaacagcttcggagatttgatgtcactggtattcacaaaaatagcattacagctctggcttggagccccaatggaatgaaattgttctctggagatgacaaaggcaaaattgtttattcttctctggatctagaccaggggctctgtaactcccagctggtgttggaggagccatcttccattgtgcagctggattatagccagaaagtgctgctggtctctactctgcaaagaagtctgctcttttacactgaagaaaagtctgtaaggcaaattggaacacaaccaaggaaaagtactgggaaatttggtgcttgttttataccaggactctgtaagcaaagtgatctaaccttgtatgcgtcacggcccgggctccggctatggaaggctgatgtccacgggactgttcaagccacgtttatcttaaaagatgcttttgccgggggagtcaagccttttgaactgcacccgcgtctggaatcccccaacagtggaagttgcagcttacctgagaggcacctggggcttgtttcatgtttctttcaagaaggctgggtgctgagttggaatgaatatagtatctatctcctagacacagtcaaccaggccacagttgctggtttggaaggatccggtgatattgtgtctgtttcgtgcacagaaaatgaaatatttttcttgaaaggagataggaacattataagaatttcaagcaggcctgaaggattaacatcaacagtgagagatggtctggagatgtctggatgctcagagcgtgtccacgtgcagcaagcggagaagctgccaggggccacagtttctgagacgaggctcagaggctcttccatggccagctccgtggccagcgagccaaggagcaggagcagctcgctcaactccaccgacagcggctccgggctcctgccccctgggctccaggccacccctgagctgggcaagggcagccagcccctgtcacagagattcaacgccatcagctcagaggactttgaccaggagcttgtcgtgaagcctatcaaagtgaaaaggaagaagaagaagaagaagacagaaggtggaagcaggagcacctgtcacagctccctggaatcgacaccctgctccgaatttcctggggacagtccccagtccttgaacacagacttgctgtcgatgacctcaagtgtcctgggcagtagcgtggatcagttaagtgcagagtctccagaccaggaaagcagcttcaatggtgaagtgaacggtgtcccacaggaaaatactgaccccgaaacgtttaatgtcctggaggtgtcaggatcaatgcctgattctctggctgaggaagatgacattagaactgaaatgccacactgtcaccatgcacatgggcgggagctgctcaatggagcgagggaagatgtgggaggcagtgatgtcacgggactcggagatgagccgtgtcctgcagatgatggaccaaatagcacacagttacccttccaagaacaggacagctctcctggggcgcatgatggggaagacatccaacccattggcccccaaagcactttttgtgaagtccccctcctgaactcactcactgtgccttccagcctcagctgggccccaagtgctgaacagtggctgcctgggaccagagctgatgaaggcagccccgtggagcccagccaagagcaggacatcctaaccagcatggaggcctctggccacctcagcacaaatctctggcatgctgtcactgatgatgacacaggtcagaaagaaatacccatttctgaacgtgtcttggggagtgtgggaggacagctgactccggtctctgccttggcagccagcactcacaagccctggcttgagcagcctccacgggatcagacattgacgtccagcgatgaggaggacatctatgcccacgggcttccttcttcatcctcagagacgagtgtgacagagctcggacctagttgctcccagcaggacctgagccggctgggtgcagaggacgccgggctgctcaagccagatcagtttgcagaaagctggatgggctactcgggtcccggctatggcatcctcagcttggtggtctccgagaagtatatctggtgcctggactacaaaggcggcctgttctgcagcgcgttgccgggcgccgggctgcgctggcagaagtttgaagatgctgtccagcaggtggcagtctcgccctcaggagcccttctctggaagattgaacagaaatctaaccgggcttttgcttgtgggaaagtcaccatcaaggggaagcggcactggtacgaagccctgccccaggcagtgtttgtggccctgagcgatgacacggcctggatcatcaggaccagtggggacctatacttgcagacaggtctgagcgtggatcgcccttgtgccagagccgtaaaggtggactgtccctacccgctgtcccagatcacagcccggaacaatgtggtgtgggcgctgacagagcagagggccctcctgtaccgggagggcgtgagcagcttctgtccggaaggcgagcagtggaagtgtgacattgtcagcgaaaggcaagctttagaacccgtctgcataacgctcggggatcagcagactctctgggccctggacatccatgggaacctgtggttcagaactggcattatttccaagaagccccaaggagatgacgaccattggtggcaagtgagcatcacggactatgtggtgtttgaccagtgcagcttatttcagacgataatccatgccactcactcggtggccacagcagcccaagcccccgtagaaaaggtggcagataagctgcgcatggcgttttggtcccagcagcttcagtgccagccaagccttctcggggtcaataacagcggtgtctggatctcctcgggcaagaatgaattccacgtcgctaagggaagtctcataggcacctactggaatcatgtggttccccgtgggacagcttctgctacaaaatgggcctttgtgttggcttctgcagctcccacgaaggaaggaagcttcctgtggctgtgccagagcagcaaggacctgtgcagcgtcagcgcccagagcgcacagtcgcggccctccacggtgcagctgcctcccgaagccgagatgcgcgcctatgccgcctgccaggatgcgctgtgggcgctggacagcctcggccaggtgttcatcaggacgctctccaagagctgccccacgggcatgcactggaccaggctggacctctcccagctaggagctgtaaaattgacaagcttggcatgtggaaatcagcacatctgggcctgtgattccaggggtggagtttacttccgtgtagggactcagcctctcaatcccagtctcatgcttccagcctggataatgattgagccacctgtccaggtaagcagaagttagctggtggaactcactcttcagtaagacagaaactgtgaggatgctggtactgggaaaaaggatctgcacagcctctagaggcctcccagcaaatgcggggagccatgcccccagggtctacacactctcgttcatcaacatcacaactggaattcgggatttgtgaagtttagagctgaacagactgttacagattatgagtcaacacgtatattttctctttcaaaataataatatttcgtttttgactttttactaagtgaatattattttttaaatctgcctatatattggaacctctattttataataataatgataataaatcagtacccagaagtataaagaaggtaaaagttactttgaaaaaaaaaaaaaaaaaaaaaaaaaaaaaaaa

SEQ ID NO: 33 - RNA, 1305 nt, Homo sapiens
  source 1..1305: mol_type = mRNA
ggccacccgcagaacagagcttccgggacccacgcctcgtttgcactgggtgctggacagccgacgcaactacaaatggggcggagctttcggcactggagcagctaatttgcatataggaatgaggtgcggctcggcttccatgggcctaatttacagatagggcggtatttctgccccttaaccgaaagtgggatacagaggacgacggtgttaggcgcctgtgtaggagtaaaatgtgtttattttgcattcaacgagagctcctgcattgcagctattttgcatatgatttgcatcttacgaagaatttgtggcaaaaaaaagctgggcgtgcgccgtaggaacctcctgctgagacgcttccggtagcggcgcgtgacccgacaggtctttcacctacctacctcagctcccacaaacacgagaagttccagcaagttcgccacttccggttctcctggctatccaatagcatcgagaggagcatccccggaagtgaggcagcggaggacgacctttttccggttccggcctggcgagagtttgtgcggcgacatgaaactgcttacccacaatctgctgagctcgcatgtgcggggggtggggtcccgtggcttccccctgcgcctccaggccaccgaggtccgtatctgccctgtggaattcaaccccaacttcgtggcgcgtatgatacctaaagtggagtggtcggcgttcctggaggcggccgataacttgcgtctgatccaggtgccgaaagggccggttgagggatatgaggagaatgaggagtttctgaggaccatgcaccacctgctgctggaggtggaagtgatagagggcaccctgcagtgcccggaatctggacgtatgttccccatcagccgcgggatccccaacatgctgctgagtgaagaggaaactgagagttgattgtgccaggcgccagtttttcttgttatgactgtgtatttttgttgatctataccctgtttccgaattctgccgtgtgtatccccaacccttgacccaatgacaccaaacacagtgtttttgagctcggtattatatatttttttctcattaaaggtttaaaaccaaaagcggtttctctttgcagcaaatatacattaaaatagagtctctgtacagccaagggctctgggccctggcttgccccatgtccctgcgcctccctggccaaacccaaaaataaatatagtgttattgctctgcagggcatagaggcagtgctctcctaccccctgaggaggctcgttgggagctgatggggaagccctg

SEQ ID NO: 34 - RNA, 14607 nt, Homo sapiens
  source 1..14607: mol_type = mRNA
ccggaggggctgtcatttgcagcgctggtcgcagccctcagctgcgccgggcggttccggctcctccctctccttgtgcctcagcgccaccatggtgctggagtcggtggtcgcggacttgctgaaccgcttcctgggggactatgtggagaacctgaacaagtcccagctgaagctgggcatctggggcggaaatgtggctttagataatctacagataaaagaaaatgccctgagtgaattggatgttccttttaaagtcaaggctggccaaattgataaattaactttgaagattccttggaagaacctttatggagaagcagttgttgcgaccctggaaggattatacctgcttgttgtccctggagcaagtattaagtatgatgctgtaaaagaagaaaaatccttgcaggatgttaaacagaaagagctatcccgaattgaagaagcccttcaaaaagcagcagaaaaaggcacacattcaggggagttcatatatggcttggagaactttgtttacaaggacatcaagcctggacgtaaacgtaaaaagcacaaaaaacattttaagaaaccttttaaaggtcttgatcgttcaaaagataagccaaaagaagccaaaaaggatacatttgtggaaaaattggcaactcaagtaataaaaaatgtacaagtaaaaatcacagatattcacattaaatatgaagatgatgtcactgatccaaagcggcctctttcatttggtgtcacactgggagagcttagtctactgactgcaaatgaacactggactccatgcatattaaatgaagcagacaaaattatatacaagcttatacgacttgatagtcttagcgcctactggaatgtaaattgcagcatgtcttaccagagatcaagggaacagattttggatcagctgaaaaatgaaattcttacaagtggaaatatacccccaaattatcaatacattttccagccaatatcagcctctgcaaaactctacatgaatccttatgcagaatcagagctcaaaacgcccaaactggattgcaacatagaaatacaaaatattgccattgaactgaccaaacctcagtacttaagtatgattgaccttttggagtcagtggattatatggttaggaatgcgccttataggaaatacaagccttatttaccacttcataccaatggtcgacgatggtggaaatatgcaattgattctgttcttgaagttcatataagaaggtatacacagatgtggtcatggagtaacataaaaaagcacaggcagttactcaagagttataaaattgcctacaaaaacaagttaacacagtctaaagtctcagaagaaatacagaaagaaattcaggacttggagaagactctagatgtttttaacataattttagcaaggcaacaagcacaagttgaggtgattcggtctgggcaaaaattaaggaaaaagtctgctgacacaggcgagaaacgtggaggctggtttagtgggttgtggggtaagaaagagtctaagaaaaaggacgaagaatcattgattcctgaaactattgatgaccttatgactccagaggaaaaagataaactcttcactgccattggttatagtgagagtacccacaacctaactttacctaagcagtatgttgcccatattatgaccctgaagttagtaagcacctctgttacgataagagaaaacaagaatattccagaaatactaaaaattcagataattggcctgggcactcaagtatctcagcgaccaggagcacaagcacttaaggtagaagcgaaattagaacactggtatataacaggtttgagacagcaggatattgtgccatcacttgtggcttcaattggtgacactacatcatccttgcttaaaattaaatttgaaaccaatccggaggatagtcctgctgaccagactctgattgttcagtcccagcctgtggaggtcatctatgatgctaaaactgtcaatgcagtggttgaattctttcaatcaaataagggattggatcttgagcaaataacatcagcaacattgatgaagctggaagaaattaaggagagaacagctacaggacttacacatattattgaaactcgaaaagtccttgatttaaggataaatctgaagccttcttatctagtagttccacagacgggtttccaccatgaaaagtcagatcttctgattttagattttggtacatttcagctcaacagtaaagatcaaggtttacagaagactactaattcatctctggaagaaataatggataaggcatatgacaagtttgatgttgaaataaaaaatgtacaactactttttgcaagagcagaggaaacctggaaaaagtgtcgatttcagcatccatcaactatgcatatattgcaacccatggatattcatgttgagttggctaaggccatggtagaaaaagacattagaatggccagatttaaagtgtcaggaggacttcctttgatgcatgtgagaatttctgaccagaagatgaaagatgtgctatatttgatgaacagtatacctttgccacagaaatcatcagcccagtctccagagagacaggtatcctcaattcctattatttcaggtggtacaaaaggtctacttggtacttcactattgctagacactgtggaatcagagtctgatgatgagtattttgatgctgaagatggagaaccacagacttgtaaaagtatgaaaggatcagaacttaaaaaagctgcagaggtcccaaatgaggagctcatcaatcttctactcaagtttgaaattaaagaagtgattttggaatttactaaacagcagaaagaagaagatacaattctagtatttaatgttactcagttaggaacagaggccacaatgagaacatttgacttaactgtggtatcttatttaaagaaaatcagcttggattatcatgaaattgaaggatccaaaaggaagccccttcacttgattagctcttctgacaaacctggattagatcttttgaaagtggagtatattaaggctgataagaatggacctagttttcaaactgcttttggaaaaactgaacaaacagttaaggtggccttttcatctttaaatctgttgctgcaaacacaagctcttgtcgcttctattaattacctcacaaccattattccatctgatgatcaaagcataagtgttgctaaggaggtacaaatttcaactgaaaaacaacaaaaaaattcaactctgccaaaagcgattgtatcctccagagatagtgacattattgatttcaggctatttgccaagttgaatgctttctgtgtcattgtttgcaacgaaaagaacaatatcgccgaaatcaagattcaaggactggattcctccctttctctccagtcaagaaagcagtcactttttgcccgactagaaaatattattgtcacagatgttgatccaaagacagttcataagaaagctgtgtcaataatgggaaatgaagttttccgttttaatttggatttgtatccagatgctactgagggggatttgtatactgacatgtccaaagtggatggtgtgctgtctctgaatgttggctgtattcagattgtctatcttcataaattccttatgtcacttctgaacttcctgaataatttccagacagccaaagagtctctgagtgctgccactgcccaggctgcagaaagggctgccacaagtgtgaaagatcttgcccagaggagttttcgtgtttccatcaatattgatttgaaagcaccggttatagtcatcccacagtcttctatttccaccaatgcagtagtggtagatcttgggttaatcagagttcataatcagttcagtctggtgtctgatgaagactacttaaatcctccagtaattgatagaatggatgtgcagctaacaaagcttacactttataggacagtgatccagccaggcatctaccatcctgatattcagctgttgcacccaattaacttggaatttcttgtaaatcggaatctagctgcatcttggtaccacaaggtgcctgttgtggaaattaaaggacatcttgattcaatgaatgttagtctaaatcaagaagatcttaatcttttatttaggatactaacagaaaatctctgtgagggtactgaagacttggataaagtgaaaccaagagtacaagagacaggtgaaattaaagagccccttgaaatctctatatcacaagatgtacatgattcaaaaaatactttaacaactggagtggaagaaattaggtctgtagacatcattaatatgctgctgaattttgaaattaaagaggttgtggttactttgatgaaaaaatcagaaaagaaaggaaggcctttacatgagctaaatgtcctgcaacttggaatggaagctaaagttaaaacctatgacatgactgctaaagcttatctaaaaaaaattagtatgcagtgctttgatttcactgactctaaaggggaacctcttcacattattaactcttctaatgtgactgacgaaccccttctgaaaatgttactgacaaaggcagacagtgatggaccagaatttaaaactattcatgacagtaccaaacagagactgaaggtttcatttgcatccttagacttagtacttcatttggaagctttactttccttcatggattttttatcatctgctgctccattctctgagccttcctcttctgagaaggaatccgagctgaaaccacttgtgggggagtccagaagtatcgctgtcaaagctgtatccagcaacatttcccaaaaggatgtgtttgatttaaagatcacagctgaattaaatgcatttaatgtctttgtctgtgatcagaagtgtaacattgcagatattaaaatacatggaatggatgcctctatttctgtgaagcctaagcagactgatgtgtttgccagacttaaagatattatagttatgaatgtagatttgcagtccattcacaaaaaggctgtctctattttgggagatgaagtctttaggttccaactgactctttatccagatgccacagaaggagaggcctatgctgatatgtccaaagtagacggcaaacttagttttaaagtgggttgtattcagattgtttatgttcataaattcttcatgtctcttttgaacttcctcaacaatttccaaactgctaaagaagctttgagtacagccacagtccaggctgcagaaagagctgcttccagcatgaaagacttggctcaaaagagtttccgccttttgatggatattaatttgaaagcaccagttattattattcctcagtcttcagtatcacctaatgctgttatagcagatctgggtttaatcagagttgaaaacaagtttagcttggttcctatggaacattattctcttcctccagtcattgataaaatgaacatcgaactcactcagttgaagctgtcaagaactattttgcaggctagcttgccacaaaatgacattgaaattttaaaaccagtcaacatgcttttgtccatacagcgaaacttagcagcagcatggtatgtgcaaattccagggatggagataaaaggaaaactaaaacctatgcaggttgctctcagtgaagatgacttgacagttttaatgaaaattttgctagaaaatcttggagaagcttcctcacaaccaagccctacacagtctgtgcaggagactgtaagagtgagaaaagttgatgtttcaagtgtacctgaccatctcaaagaacaagaagattggacagactcaaagctctctatgaaccagattgtcagtctccaatttgactttcactttgaatctctttccattatcctttataacaatgatatcaaccaggaatctggagttgcatttcataatgacagtttccaacttggtgaactcagactacatcttatggcctcctcagggaagatgtttaaggatggctcaatgaatgtcagcgttaaacttaagacatgcacccttgatgatctcagagaaggaattgagagagcaacatcgagaatgattgacagaaagaatgaccaagataacaacagttctatgattgatataagttacaaacaagacaaaaatggaagtcaaattgatgctgttcttgacaagctgtatgtatgtgccagtgtggaatttctgatgactgtggcagatttctttatcaaagctgtgcctcagagtccagaaaatgtggcaaaagaaacacagattttaccaagacagactgccacagggaaggtcaagatagagaaagatgactctgttagaccaaatatgactttaaaggccatgatcacagatccagaagtggtatttgttgccagcctgacaaaggctgatgctcctgctctgacagcctcgtttcagtgcaacctttctctgtcaacatccaaactcgaacagatgatggaagcttctgtgagagatctgaaagtgctcgcttgcccttttctcagagaaaagagagggaaaaacattaccacagtcttgcagccctgttctttatttatggaaaaatgtacgtgggcttcaggaaagcaaaatataaatattatggttaaagaatttataattaagatttcacccataattcttaatactgtgttgacaatcatggctgcattgtctccaaaaacaaaagaagatggatccaaagatacgtctaaggaaatggaaaatctttggggtatcaaatcgattaatgattataacacttggtttcttggtgttgacacggcaacagaaataacggaaagcttcaaaggcattgaacattcactgatagaggaaaattgtggtgttgttgtagaatccattcaagttaccttagaatgtggccttggacatcgaactgtacctttattattggcagagtctaagttttcaggaaatattaaaaattggacttctctaatggctgctgttgctgacgtgacactacaggtgcactattacaatgagatccatgctgtctgggagccactgattgagagagtggaggggaagagacaatggaatttaaggcttgatgtaaagaagaacccagttcaggataaaagtttgctgccaggagatgattttattcctgagccacaaatggcaattcatatttcttcaggaaatacaatgaatataacaatatccaaaagttgtcttaatgttttcaacaatttagcaaaaggtttttcagagggcactgcttctacttttgactactctttaaaggacagagctccttttacggtaaaaaatgctgtaggtgttcccattaaggtgaagcccaattgtaatctcagagtaatgggcttccctgagaaaagtgatatttttgatgttgatgctggccagaatttggaactggagtatgccagcatggtaccttcaagtcaagggaacctatctatattgagccgtcaagaaagctccttcttcactctgaccattgtacctcatggatatacagaagttgcaaatatccctgtggccagacctggacggcgattgtataatgtacggaatcccaatgccagtcattctgactctgtcttggtacaaattgatgcaactgaagggaataaagtaattacccttcgctctcctctacagatcaaaaaccatttctccattgcatttatcatctataaatttgttaagaatgttaagctattggagcgcattgggatagccagacctgaagaggagttccatgttcctttagattcatatagatgtcaattgtttatccagccagctggaatcttagagcatcagtacaaagaatctaccacttatatttcctggaaggaagaacttcataggagcagggaagtcagatgcatgttgcagtgtccatcagtagaagtcagcttcttacctctcatagtgaatacagttgctctgcctgatgaattgagctacatatgtacacatggggaagactgggatgtagcttacattattcatctttatccttctctcactttgcggaatcttctcccatattccctaagatatttacttgagggaacagcagaaactcatgagctggcagaaggcagtactgctgatgttctgcattcgagaatcagtggtgaaataatggaattagtcctggtgaaataccagggcaaaaactggaatggacatttccgcatacgtgatacactaccagaattctttcctgtgtgtttttcttctgactccacagaagtgacgacagtcgacctgtcagtccacgtcaggagaattggcagccggatggtgctgtctgtctttagtccctattggttaatcaacaagactacccgggttctccagtatcgttcagaagatattcatgtgaaacatccagctgatttcagggatattattttattttctttcaagaagaagaacatttttactaaaaataaggtacaattaaaaatttcaaccagtgcctggtccagtagtttctcattggatacagtgggaagttatgggtgtgtgaagtgtcctgccaacaatatggagtacctggttggtgttagcatcaaaatgagcagtttcaacctttcacgaatagttaccctgactcccttttgtaccattgcaaacaagtcatcattagaactagaagttggcgagattgcatctgatggctcaatgccaactaataaatggaactatattgcttcttcagagtgccttccattttggccagaaagtttgtcaggcaaactttgtgtgagagtggtgggctgtgaaggatcttccaaaccattcttttataaccgacaggataatggcactttattgagcttagaagatctgaatgggggtatcttggtggatgtaaacactgccgaacattcaactgtcataactttttctgattaccatgagggatctgcacctgccttgataatgaaccatacaccatgggacatcctcacatacaaacagagtgggtcaccagaagaaatggtcttgctgccaagacaggctcgactttttgcctgggcagatcctactggtaccagaaaacttacatggacatatgcagcaaatgttggggaacatgatctgttaaaggatggatgtggacagtttccatatgatgcaaacatccagatacactgggtatcatttctggatgggcgccagagagttttgcttttcaccgatgatgttgccttggtttccaaagcactgcaggcagaagaaatggaacaggctgattatgaaataaccttgtctctccacagtcttgggctttcactggttaacaatgaaagcaagcaggaagtttcctatattgggataaccagttctggtgttgtttgggaggtgaaaccaaagcagaaatggaagccatttagtcaaaagcagataatcttattggaacaatcctatcagaaacatcaaatatcaagagaccatggctggattaagctagataataattttgaggtcaattttgataaagatccaatggaaatgcgcctccctattcgtagccctattaaacgagactttttatcaggaattcagattgaatttaagcagtcttctcaccagagaagtttaagggccaggttgtactggcttcaggttgataatcagttaccaggtgcaatgttccctgttgtatttcatcctgttgcccctccaaaatctattgctttagattcagagcccaagcctttcattgatgtgagtgtcatcacaagatttaatgagtacagtaaagtcttacagttcaagtattttatggtcctcattcaggaaatggccttaaaaattgatcaagggtttctaggagctattattgcactgtttaccccaacaacagaccctgaagctgaaagaagacggacaaagttaatccaacaagatattgatgctctaaatgcagaattaatggagacttcaatgactgatatgtcaattcttagtttctttgaacatttccatatttctcctgtgaagttgcatttgagtttgtctttgggttccggaggtgaagaatcagacaaagaaaaacaggaaatgtttgcagttcattctgtcaacttgctgttgaaaagcataggtgctactctgactgatgtggatgaccttatattcaaacttgcttattatgaaattcgatatcagttctacaagagagatcagcttatatggagtgttgttaggcattacagtgaacagttcttgaaacagatgtatgtccttgtattggggttagatgtacttggaaacccatttggattaattagaggtctgtctgaaggagttgaagctttattctatgaacccttccagggtgctgttcaaggccctgaagaatttgcagaggggttagtgattggagtgagaagcctctttggacacacagtaggtggtgcagcaggagttgtatctcgaatcaccggttctgttgggaaaggtttggcagcaattacaatggacaaggaatatcagcaaaaaagaagagaagagttgagtcgacagcccagagattttggagacagcctggccagaggaggaaagggctttctgcgaggagttgttggtggagtgactggaataataacaaaacctgtggaaggtgccaaaaaggaaggagctgctggattctttaaaggaattggaaaagggcttgtgggtgctgtggcccgtccaactggtggaatcgtagatatggccagtagtaccttccaaggcattcagagggcagcagaatcaactgaggaagtatctagcctccgtccccctcgcctgatccatgaagatggcatcattcgtccttatgacagacaggaatctgagggctctgacttacttgagcaagaactggaaatacaggaataaatgtttcctaaactactacttgatttcatccttaaaaatcaaaacaaactgtggtgttaattgactgtgtgtgaattccattgtcaattttaatgaaattttctttaaaactctcacctccatctgaacttttcatagtagtgggattgactacaaataaaaacttgtggtattcctggtaatactgtccagaaataagagattagtataaaatattaaaggatgcagagaatcagctctcttctgcgtttaatagatgaaagcctttattgagctcagaagcagatactgttactatcatttcgaaaattttatcttatggtgttcatgtgcatttcaggtaaaattgaaaaacaggacaattattatgtccaattaatatgtttatgtttgtgagtcttgatgatggaattacatagctttctgtttcacaaatggctctaaatttgcttaagttacgggactattacctggagcatctgctttaataattgaattgtcagttgctctgagcctgcccttagacctcaagtaataaatagttggcacatgaattttgaggatatgtttcctcttccctctttttcctatttaaccccttggtactgttgctaaataaatgatagccattttataattatgttatatacattttcagcctttagcatttctgcttttcaaaaattgaatctccttgttggttatgcttatttcataattattagttttaattaatgtagatagaagttgaacatgtaattaggcaaattgctgtgtggcacttgaatacatagatttctttattttcaaaaaccaaccttttgcttttaaatccttagagagggtttattatcttagagaaaaaataattataatcattatttttgaaattagtatcctcttaattctcaacataagttatgtttcaatttcttttttttgtaataaatgatggaaatgtttaacaatgtcttatctagcaactttcatgcttctcctcagaaatgaagccaaagtataaacttagatttaatgtgttgtatatttgaagagaatgaaactattaacatataattgttcagttggattatgtattttaaggattgcagttatcaaaataataaattgaatgttttatgtttaaccactttaaagaagaaagactgacatccaaaaaccagcgtgtgctagatatacaaaggaaattacttctgtccttaagggaccaagtataacaaaacatgtaactgttaaaagtagctgacaaacctttcttgtgcctagataatttagcattggcaaaaatgtcaccacatgcagttttctaggagagtcaagcacaaataactaattcaagatgctgacttaaatcatctccaatagttacccttcctgagattctaaagtaacaatttttaattttactggttatattgctgttttactgagacttacttttaagaacccctgtaacttaagattttttcttaattgttttgtttagctctgttattaattttttccttgtgatatctttttataactctctgtcaaaaagcacaaaacttcaagaaacttttaattattttgtctgaacatataatcttgtctgatttcttagtttttattaagatatcagacaacttttaaaactttagtgcattattataattactggaagaaaaagaatgattatacactaatgagaggacttggtagtttttgtcgtggatgtcaagtgtgggcatggataattgaaatatttaggctatttcattctttgcccatcttgctgtgatcagttagttgggtaaaaatatttattgattatttagactgtactggatatacaaaagaagccttctgtccttaagggaccgagtaaaacaaaacatggaaatattaaagagtattagagtataaaagtatatctttttagccctttgtaatatggccaaattctaaataatttatttggggatcttttgatcctcatgttcctttttctcctaagtactactttgtattctttaatatgcagctttgagagttactgaatcatatattatatttccatgagatgtactattctacttatcctctaatcttcatatatatatacacacacacatatatatacacatacatatatacacacgtacatatatgtacacatacagatatacatacacacaaacacatatatacacacatacatatacacacatatatatacacatacaaatatacacatatatacacatacatatatatacacacatacaaatatacccatatgtacacatacatatatacacatacatatatacacacacatatacacacatatatacgcaaacatacacatatttacacatacatatatacatacattatatgtatgtatatatagtcatttaatactcattttggttcacatacttatgatcatgcaacgtttaaaacagcatttcttgctttttagttttagttatatttttccatgttcttagaaatgcctcattaacatttttaattcttgtattgccatctattgaggtgacattacattgtgtttttatctcgtcttaattcatgacattaaattattctactaacagtaataatgctgtaataaacatcattatagattttgcttttttatatcttgtttgctttttcatatttccttagaatttacttgaaaaaattgaattactgggtaaagggcttttgcaaagtattgttaaattcctcgagttgcatttttggaaaggggacgtgaatattttatcaactaatttggtctccctgctgccattagtgactgaatatcttaatctgaatctcagagtgtagtgggtttttagtagtgctgaagacaagttttctaaagtgtattatggtgataaattatattttaaaaactgtcaatggcttgaagcacaatagcctaataactaacgaaaatacatacaagatagaaagtgggtagtatttcttgtacttgcatttcagatctaaatattttaacatatttaaatttcaagctgcagataaatgcattacattattaaattcatttcccattttctctttgaagaaattaaggcaaaagtgttaaaagattttaactaattcgcacaagtgaattgtgaaacaagtagctattgctgtgaaatctgcactcctctctgagactcattctgaagatgagatcccagttctttgtggattcctcttccttattcatggctttttgcaattgtcaaggaatgactaggtaccaagcaactttaaaaaatgtatatttaagcattgaaataatatcaaatgtgatttctctgcttgtggttatattgattatattatccttttaataatattggcattatattcttggtcgtaaaatgtcaaggtcttatttattcagtatatttatgttctgtattttcatatatattatctattttcagccatgcattatatataatgtcagtaatagtatttcattagcattcattataaaaaaactcgtttttaatatttgactaattcaagtcacagtacttttgagatagctgaaaaggaaaataaatgtgttttaatgtgctactaaaaaaaaaaaa

SEQ ID NO: 35 - RNA, 14329 nt, Homo sapiens
  source 1..14329: mol_type = mRNA
gcggccgcagaatcgagctcgggccccggcccccggcccgcggcgcggggctcccgggccccgccgcggacgtcgcgccggtcgccccttccccgtagcccgtgcgccctcggcgcggagccccggcccgccgcggtcccgtctcctgggcctgtcccgcccgcgccctccgccggccctcaggtataatacttctccacgtctgcttcaggaagaaagtgcctgccattcttatcatttctaagcaggttcatgccagcccagaacagagaatcagctggagcccagatttcaagttttgagtaaaataccttcaagcgaatgggccctattgtgctcacacattcagaacctgttacccaaggaattccctaaagaattagaagtgcgtctcaccaaccagccaagatgaacatggtgaagaggatcatggggcggccgaggcaggaggagtgcagcccacaagacaacgccttaggactgatgcacctccgccggctcttcacggagttgtgccatcctccccggcacatgactcagaaggaacaagaagagaaactgtatatgatgctgccagtgtttaacagggtttttggaaatgctccgccgaatacaatgacagaaaaattttctgatcttctgcagttcacaacacaagtctcacgactaatggtgacagaaattcgaaggagagcatcaaacaaatccacagaggctgcaagtcgggccatagttcagttcctagagattaatcagagtgaagaagccagtagaggctggatgcttctaacgacaattaatttgttagcttcctctggtcagaaaaccgtggactgcatgacaacaatgtcagtgccttccaccctggttaaatgtttatatctgttttttgaccttccacatgtgcctgaggcagttggaggtgcacagaatgagctacctctagcagaacgtcgaggactactccagaaagtttttgtacagatcttagtgaaactgtgcagttttgtttcccctgcggaggagctggctcagaaagatgatctccagcttctattcagtgcaataacctcttggtgccctccctataacctgccttggagaaagagtgctggagaagtcctcatgaccatatctcgtcatggtcttagtgtcaatgtagtgaagtatattcatgagaaagagtgtttatctacatgtgttcagaatatgcagcaatcagatgacctgtctcccctagaaattgtcgaaatgtttgctgggctttcttgtttcctcaaagattccagcgatgtttcccaaacacttctggatgattttcggatatggcaaggatataattttctttgtgatctcttgcttagattggaacaagcaaaagaggcagaatccaaagatgccttgaaagatctggttaatctgataacttccctaacaacatatggtgtcagtgaactaaaaccagctggtattaccacaggggcaccctttttattgcctggatttgcagtacctcagcctgcaggcaaaggtcacagtgtgagaaacgtccaggcctttgcagttcttcagaatgcatttttaaaagcaaaaaccagcttccttgcccaaatcatccttgatgctatcacaaatatttacatggctgacaatgccaattacttcatcctagagtcacagcacacattgtcacagtttgcagagaagatttctaaactcccagaagtacaaaacaaatactttgagatgctggagtttgttgtttttagcttaaattatataccttgtaaagaacttattagtgtcagtatcctcttaaaatctagctcttcttatcactgtagcattattgcaatgaaaacacttcttaagtttacaagacatgactacatatttaaagacgtgttcagggaggttggccttttggaggtcatggtaaaccttttgcataaatatgctgccctgttgaaggatccaactcaggcactaaatgaacaaggggactcaagaaataatagttcagttgaagaccaaaaacacctggctttattggttatggagaccttgacagtgcttcttcaaggatcaaacacaaatgcaggaatttttcgagaatttggaggtgcaagatgtgcacataatatagtaaagtaccctcaatgccggcagcatgccttgatgactatccaacagctggtgctctccccaaatggggacgatgacatgggcactctcctggggctaatgcattcagccccaccgacggaattgcagttgaagactgatattttaagggccctcctgtcggtccttcgagaaagccatcgttcaagaacagtttttaggaaagttggaggatttgtgtacattacatccttgctcgttgctatggaaagatctttgagctgtccacccaagaatggctgggagaaagtgaaccagaatcaagtgtttgaacttcttcacactgtgttctgcacgttgactgcagcaatgcgctatgagccagccaactctcatttcttcaaaacagagattcagtatgagaagttggcagatgctgttcgatttcttggctgcttctcagacctaagaaaaataagcgccatgaatgtcttcccctcaaatacacagccatttcaaagacttttagaggaagatgtaatctcaatagaatcagtgtcacccacgttacggcactgcagtaaactttttatttatctttacaaagtagccacagattcttttgacagtcgtgcagaacagatccctccttgcctgacaagtgagtcttctctcccctctccttggggtacaccagctttgtccaggaaaaggcatgcatatcattctgtttcaactccccctgtttaccctcctaaaaatgttgccgacctgaaactacatgtgacaacttcatctctgcagagttctgatgcagtcatcattcatcctggagccatgcttgccatgctggacctactggcctctgttgggtcagtgacacagccagaacatgctttggatcttcaacttgccgtggcaaatattttacaatccctggtgcacacagaaaggaaccagcaagtcatgtgtgaagctggtcttcatgcacgactgctgcagaggtgcagtgctgcattggctgatgaggaccactcactgcacccgcccctgcagcggatgtttgaacgattagcctctcaggctctggaacccatggtgttgagggagtttttacgtttggcaagtcctttaaattgtggtgcctgggacaaaaaactgctaaaacaatatagggtccacaaaccaagttcactgagttatgaaccagaaatgagaagtagtatgatcacatctctggaaggtctgggtactgataatgtttttagcttacatgaagataaccattaccggataagcaagagcctggtaaaatctgcggaaggaagtactgtacccctgaccagggtgaagtgtctggtctccatgacaaccccacatgacatcagacttcatgggtcatcagttactccagcttttgttgaatttgacacatcacttgaagggtttggatgtctttttttgcccagtttggcccctcataatgctcctacaaataataccgtcacaacaggtcttattgatggggctgtggtcagtggcattggttctggtgaaagattcttccctcctccctccggcttaagttactctagctggttttgtattgaacattttagttctcctccaaataaccaccctgtcagacttcttactgttgtgcgccgagcaaattcttctgagcaacattacgtgtgccttgcaatagttctatcagcaaaagaccgatctctgattgtttccaccaaagaggaactcctccaaaattatgttgatgattttagtgaagagtcctcattttatgaaattctcccatgctgtgctcgctttcgatgtggagagcttatcattgagggacagtggcatcatttggtcctggtaatgagcaaaggcatgttgaaaaacagtactgcagccctttatattgatggacagcttgttaacactgtaaagcttcattatgtccacagtactccagggggttcaggttcggcaaatccaccagtggtgagcacggtctatgcctacattggtactccacctgcccaacgccaaattgcctcattggtttggcgcctgggacccacacattttctagaagaagttttaccttcttcaaatgttactaccatttatgaacttggaccaaattatgttggaagctttcaggctgtatgtatgccatgtaaagatgcaaaatccgaaggggtggtgccatcccctgtgtcattagtaccagaggagaaagtgtcatttggcctctatgcactctctgtgtcgtctctaacagtggcaagaatccggaaagtgtataacaaattggatagcaaagccattgctaagcagttaggcatttcctcacatgagaatgccactcctgtgaagttgatacacaattcagcaggacatcttaatggatctgcacggacaattggggccgctctgattggatacttgggagtaagaacatttgtccctaagcctgttgccactactttgcagtacgttggtggagctgcagccatcctgggcctggtggccatggcctctgatgtggaagggttatatgcagcagtcaaggccctggtttgtgtggtcaagagtaacccactagccagcaaagaaatggaaagaatcaagggctaccagttgctggcaatgttgcttaagaagaaacgttcccttcttaacagccacatcctccatctaactttttctttggtgggaactgttgatagtggacatgagacctccattattccaaattcaactgctttccaggacctcctctgtgattttgaagtctggctccatgcaccatatgaacttcatctttccttatttgaacactttattgaactgctcacagagtccagtgaagcctcaaagaatgccaaattaatgagagaattccagttaatcccaaagctgctcctgactcttcgagatatgtctttatcccagcctactattgctgctattagtaatgtcctgagcttcttactgcaaggttttcctagcagcaatgatctgctcagatttgggcagtttatttcttctactttgccaacctttgcggtttgtgagaaatttgtagtaatggaaataaataatgaagagaagcttgacactggaactgaagaggagtttggaggtcttgtatcagctaatcttatacttttgaggaacagacttctggatatcttgctaaaactaatttatacatctaaagaaaagacaagcattaatttgcaagcttgtgaagaactggtgaagacactgggttttgactggatcatgatgtttatggaggaacacttacattccaccacagttacagcagccatgaggattcttgttgtcctactaagtaatcagtctattctcatcaagtttaaagaaggactcagtggtggaggatggcttgaacagacagattctgtcttaactaataagattggaactgtattaggattcaacgtgggcagaagtgctggtgggagatcgacggtcagggagattaaccgagatgcttgtcattttcctggttttccagtccttcagtcattccttcctaaacacactaatgtccctgccctctattttctcctcatggccttgtttctgcagcagccagttagtgagctgcctgagaacctgcaggtcagtgtgcctgtcatcagctgccggagtaagcagggttgccagtttgatttggattccatttggacattcatctttggagttcctgcctccagcggaactgtggtctcttctatccataacgtatgcacagaagctgtttttttattattgggaatgctccgcagcatgctgacttcaccttggcaatcagaagaagagggatcttggctccgagaatatcctgtgaccctgatgcagttcttcagatatttgtatcacaacgtgccagaccttgcctccatgtggatgagccctgacttcctgtgtgcattagcagccaccgtcttccccttcaatattcgcccttactcagagatggtgactgaccttgatgatgaagttggatctccagcagaagagtttaaagcgtttgcagcagacacagggatgaacaggagccaatcagagtactgcaatgtgggcaccaagacatatctgaccaatcacccggctaaaaagttcgtttttgacttcatgcgggtcttaatcatagacaacctctgtctcactcctgccagcaagcaaactccactaattgatcttttgttggaggcttcccctgaaaggtctacaagaactcagcaaaaagaatttcaaacttacattttggatagcgtgatggaccatttgcttgcagctgatgtgttattaggggaagatgcatctctgcctattaccagtggaggaagctaccaggtattggtgaacaatgtgttttatttcacacagcgtgtggtggacaagctttggcaaggcatgttcaacaaagaatctaaacttcttatagattttataattcaactaattgcacagtcaaagagaagatcacagggattgtcactggatgcagtgtatcattgcctcaataggaccatcttgtaccagttctcacgggcacacaaaaccgttcctcagcaagtagctctgcttgattcactcagggtcctcactgtaaacagaaacttgatcctgggacctgggaaccatgaccaagaattcattagctgtctggcccactgcttgataaatctacatgttggaagcaacgtggatggatttggactggaagcagaagcccgcatgaccacatggcacattatgatcccctcggacattgaaccagatggtagttacagccaagatattagtgaagggcgtcagcttctcataaaagctgtcaacagagtttggactgaactgatacatagtaagaaacaagtcttagaggaacttttcaaagtaactctacctgtgaatgaaaggggccacgtggacatagctacagcaaggccactcattgaagaagctgccctgaagtgctggcagaatcatttggcccatgaaaagaaatgcataagtcgaggagaagctttagcgcccaccacacagtccaaattatcccgtgtcagcagtggctttggtctttccaagttaacaggatcaagaaggaatcgaaaagaaagtggtcttaataaacacagtctttccacccaggagatttcgcagtggatgtttactcacattgctgttgttcgtgacttagtagatacacaatataaagaatatcaggagcgtcagcagaatgccctgaagtacgtgacagaagagtggtgtcagatcgagtgcgagctgttgagggagcgggggctgtggggccctcccatcggctcccacctcgacaagtggatgctggagatgacagaagggccctgcaggatgaggaaaaagatggtgcgaaatgatatgttttataaccattacccttacgtgccagaaactgagcaagagacaaatgtggcgtctgagatcccaagtaaacagcctgagacacccgatgatattcctcaaaagaaacctgctcgatatagaagagccgtaagttatgacagtaaagagtactacatgcgactggcctctggcaatcccgccattgtccaagacgccattgtggagagttcagaaggtgaagctgctcagcaagaaccagagcatggggaagacactattgctaaagtcaaaggtttggtcaagcctcctctaaaacgctcccgatctgcacctgatggaggagatgaggagaaccaggagcagctacaagaccagattgctgagggcagctccatagaagaggaggagaaaacagataatgctaccttactgcgcctgttagaggaaggagaaaagatccaacacatgtaccgctgtgctcgagtccagggcctagataccagtgaggggctccttctttttggtaaagagcatttttatgtgattgatggatttaccatgacagcaaccagggaaataagagatattgaaaccttacctccaaatatgcatgagcctattattcctagaggagccaggcaaggccctagtcaactcaagagaacatgcagcatttttgcatatgaagatatcaaggaagttcataaaaggagatatctcctgcagcctattgctgtggaagttttctctggagatggacggaattacctccttgcttttcagaaaggaatcagaaacaaagtctatcaaaggtttttggctgtagtgccatctctaacggacagttcagaatctgtatctgggcaacgaccaaacacgagtgtggagcagggatctgggttacttagcactttggttggagagaagtctgtgactcagagatgggagagaggtgaaatcagcaacttccaatatttgatgcatttgaacactttggctggcagatcatataatgatctcatgcagtatcctgtcttcccctggatccttgcagattatgactcagaggaggtggatcttactaatcccaagacgtttagaaacctggctaagccaatgggagcacaaacagatgaacgattagctcagtataagaagcggtataaagactgggaggatcctaatggagaaactcctgcataccactatgggacccactattcatctgcaatgattgtggcctcataccttgtaaggatggagcctttcacacagatattcttaaggctacagggtggccactttgacctggctgaccggatgtttcacagtgtgcgcgaggcctggtattcagcgtcaaagcacaatatggcagatgtaaaagaacttatcccagagttcttttatttaccagaattcctgttcaattccaacaactttgatctaggctgtaaacaaaatggcaccaagcttggagatgttatccttccaccctgggcaaaaggggacccacgagaattcatcagagtccatcgtgaggctttggagtgtgattacgtgagtgcccatctacatgagtggattgacttaatcttcggttataaacagcaaggccctgctgcagtagaagctgtaaatgtcttccatcatcttttttatgagggtcaagtggatatctacaacatcaatgacccactaaaggagacagccacaattgggttcattaataacttcggtcagatccctaaacagttatttaaaaaacctcatccaccaaagcgagtgagaagtcgactcaatggagacaatgcaggaatctctgtcctaccaggatctacaagtgacaagatcttttttcatcatctagacaacttgaggccttctctaacacctgtaaaagaactcaaagaacctgtaggacaaatcgtatgtacagataaaggtattcttgcggtggaacagaataaggttcttatcccaccaacctggaataaaacttttgcttggggctatgcagacctcagttgcagactgggaacctatgagtcagacaaggccatgactgtttatgaatgcttgtctgagtggggccagattctctgtgcaatctgccccaaccccaagctggtcatcacgggtggaacaagcacggttgtgtgtgtgtgggagatgggcacctccaaagaaaaggccaagaccgtcaccctcaaacaggccttactgggccacactgataccgtcacctgcgccacagcatcattagcctatcacataattgtcagtgggtcccgtgatcgaacctgtatcatttgggatttgaacaaactgtcatttctaacccagcttcgagggcatcgagctccagtttctgctctttgtatcaatgaattaacaggggacattgtgtcctgcgctggcacatatatccatgtgtggagcatcaatgggaaccctatcgtgagtgtcaacacgttcacaggtaggagccagcagatcatctgctgctgcatgtcggagatgaacgaatgggacacgcagaacgtcatagtgacaggacactcagatggagtggttcggttttggagaatggaatttttgcaagttcctgaaacaccagctcctgagcctgctgaagtcctagaaatgcaggaagactgtccagaagcacaaatagggcaggaagcccaagacgaggacagcagtgattcagaagcagatgagcagagcatcagccaggaccctaaggacactccaagccaacccagcagcaccagccacaggccccgggcagcctcctgccgcgcaacagccgcctggtgtactgacagtggctctgacgactccagacgctggtccgaccagctcagtctagatgagaaagacggcttcatatttgtgaactattcagagggccagaccagagcccatctgcagggcccccttagccacccccaccccaatcccattgaggtgcggaattacagcagattgaaacctgggtaccgatgggaacggcagctggtgttcaggagtaagctgactatgcacacagcctttgatcgaaaggacaatgcacacccagctgaggtcactgccttgggcatctccaaggatcacagtaggatcctcgttggtgacagtcgaggccgagttttcagctggtctgtgagtgaccagccaggccgttctgctgctgatcactgggtgaaggatgaaggtggtgacagctgctcaggctgctcggtgaggttttcactcacagaaagacgacaccattgcaggaactgtggtcagctcttctgccagaagtgcagtcgctttcaatctgaaatcaaacgcttgaaaatctcatccccggtgcgtgtttgtcagaactgttattataacttacagcatgagagaggttcagaagatgggcctcgaaattgttgaagattcaacaagctgagtggagaccatggtctgtagaccccttcccgattctcctgtcccagcttggaaggcattgaaaacagtctccgtttacacatctcttcataccacgtgtttgaagtgttaaaattcaaagggatcattgaataaaacgggtgtagagtacaggaatggggcagacgcgattcaggtgaacagcacaagaagaatatgaggtggttcctaggagcaacactttcgacctccagttctccctgatgacagtagctgtctccaagagaaaaatcctcacttattaactctcttttcttgcatctcatttttatagagctactcatccttatttggaaaaaccaacaacaaaaaaggcttttagaaaatggttgtaaatctgacttctttgcaagtaactatgtatattgtaaatagatataaaaggccttttttctaaataaggacttaactgcctgtaacatgaaacttcaaactaaaccactaactcaatgaactacttatggtttgtctgacatccctcacttaccaattaattataaatatgtttttttaaatccccaaagacattatctgtggtctttttttcctttcaagctcagcctgtgtgcctgatgtcatttctttcaagttgcccacagtatctccacttaaactaggctagtaaccaaaataatgtggaccttctttaggaaacagtgtgggagaataggagtccagccgtaagataaactggaaatatttgggcgtcttgtacctggctacgcaccacctcagtgttgttcctacataaacagggccccttttaaacttgtatgtggactgctgtttggtcaaagaataccttcttagcattgcagaaaggtggtcagatgaccagtgtagtgcaggaaacagccctgtctcaactaatggaaatatatttgcatgtaacccaaaattagcttatcttgcatagaacataataagtatgtgtctttggtgacactaatgttctactatagcttattttcaaacaaggggtaaaaaaaggaaagaaagaagtgtacagaattaacatataaactttgttgtaaaactgaatcatgtcagaactgcttaaaattaacctttaccatttaatgtcatctacctgaaaacagtgagatttatactgtatcaatgtctatttttttgtttttgctatgaatataattacagtattttaatatttagttatttaatttgttctactagttggatacagaacacacaaatccagggggattaaagctggaaggggctaagagattagtttacagagaaaaggcttggtggtgggatttttttaaatgtgtgttatgtacatatatatatatatataatatatattaaaaatgaaacaattaatctagattttaacattttcagaaacttagtgataacattatgaacaattctaaaagccctgtgatttgaaaaatatagaatcattaatggcccaagataggccttcacaccttcacaggtgcgaaaggaaaggccttcacaccctcacagaggcatcatgcaaaggacagcggctttggcttttccaattttccatctttaggccctggtgagaggcacacttatgcactaaaatgcacatatatgcacatgcattcaaaaataggcatttggtacaatggtgatcttgtacctgatgggctgaaaccagcttaagaacaaatttgttcttcctgatatgataactaggtctccaagagaaaatagaaaggctgctttagtgccttacgcttactaaatttaaatctttatttacctgggtttgagcctacagtctatttatgattacatatcaaaattgattaaaacacttccatttctaaaagttcaaatatacttgttaataaaaggattatcggcattaatactttaatttaaagaaaagttgtgttctgttttcctttctgtgtcttactccccccacactctccctcccccatcaccatcttcaattctaataaataatgctgatgttcaacagttgcagaaattgtgctattatgtaactgtgggccttgcccctgtctggccctctagatgatttgtagcagtgttattctacactttttaaaagaagcgtcctccttttgtccatgaatcatgtttaccccatacccagtggcagaggtgttctttaaagacttgaatatatgaatgtgtgtgtgtagttacttaaaggttattcctctttgtaataggaaactatatgggatgaacacttttaaactttccgacacaacttccattactaactttctaacagaacttccataactagaaggtggaaaccaaaaccctcatggtagtatttcctctggcagctggtgctgtgggcaactgttttgttcaatcgggtttcttttctttttgcctctaatgcagaaatcaacagaatcactcacacatacaagtacactcacatacataaactaattatttctctggatatctttctgtgttccatgtaaatttatttaccaacatctattgtcaacatgtacatctaccttagtatggtctgcattctttttctgagagtacctcatagggctcctgcctgatctttgtagtttgttcattcatccatccacctgttcatttgttcatccatgtattctaacatttctatgtagtgtgcaactctaatgtcatgcttttgaagaagagaatagctgcccatagcagccatccgtctggataatagcaaaacactctagataagttattttgcactttcttatgtataaagttggtagaaacttatttttgctttgtatcatttaaatacattttgttttggtaaatgaactgtgtataaaatatttatgccgttaaaactgtttttagaaagtatttttaatttcagcaagtttggttacttgttgcatgactcttaacacagctgactttttgtgtcagtgcaatgtatattttttgtcctgttattaacttgtaagccctagtaatggccaattatttgtacagcaacagaagtaaattgaagatactggctaagactggattgattgtggacttttatactatattgcagaaaccaatatctgtttcttggtggttatgtaaaagacctgaagaattactatctagtgtgcagtctgtgatatctgaatgttcattgtatatttgtctctgatgcaaaaaggtagagtaacacaattacaatacatgattaaatgcaatagtccaggtacttaagtaatttttttttcatttcaaataaatacctatttaccaccaaaagaaagaaaaaaaaaaaaaa

SEQ ID NO: 36 - RNA, 4826 nt, Homo sapiens
  source 1..4826: mol_type = mRNA
gggcgcgggcagctctgcgtccgaagctgctccgacgccgtcgctgggaccaagatggacctcccggcgctgctccccgccccgactgcgcgcggagggcaacatggcggcggccccggcccgctccgccgagccccagcgccgctcggcgcgagccccgcgcgccgccgcctgctactggtgcggggccctgaagatggcgggcccggggcgcggcccggggaggcctccgggccaagcccgccgcccgccgaggacgacagcgacggcgactctttcttggtgctgctggaagtgccgcacggcggcgctgccgccgaggctgccggatcacaggaggccgagcctggctcccgtgtcaacctggcgagccgccccgagcagggccccagcggcccggccgccccccccggccctggcgtagccccggcgggcgccgtcaccatcagcagccaggacctgctggtgcgtctcgaccgcggcgtcctcgcgctgtctgcgccgcccggccccgcaaccgcgggcgccgccgctccccgccgcgcgccccaggcctccggccccagcacgcccggctaccgctgccccgagccgcagtgcgcgctggccttcgccaagaagcaccagctcaaggtgcacctgctcacgcacggcggcggtcagggccggcggcccttcaagtgcccactggagggctgtggttgggccttcacaacgtcctacaagctcaagcggcacctgcagtcgcacgacaagctgcggcccttcggctgtccagtgggcggctgtggcaagaagttcactacggtctataacctcaaggcgcacatgaagggccacgagcaggagagcctgttcaagtgcgaggtgtgcgccgagcgcttccccacgcacgccaagctcagctcccaccagcgcagccacttcgagcccgagcgcccttacaagtgtgactttcccggctgtgagaagacatttatcacagtgagtgccctgttttcccataaccgagcccacttcagggaacaagagctcttttcctgctcctttcctgggtgcagcaagcagtatgataaagcctgtcggctgaaaattcacctgcggagccatacaggtgaaagaccatttatttgtgactctgacagctgtggctggaccttcaccagcatgtccaaacttctaaggcacagaaggaaacatgacgatgaccggaggtttacctgccctgtcgagggctgtgggaaatcattcaccagagcagagcatctgaaaggccacagcataacccacctaggcacaaagccgttcgagtgtcctgtggaaggatgttgcgcgaggttctccgctcgtagcagtctgtacattcactctaagaaacacgtgcaggatgtgggtgctccgaaaagccgttgcccagtttctacctgcaacagactcttcacctccaagcacagcatgaaggcgcacatggtcagacagcacagccggcgccaagatctcttacctcagctagaagctccgagttctcttactcccagcagtgaactcagcagcccaggccaaagtgagctcactaacatggatcttgctgcactcttctctgacacacctgccaatgctagtggttctgcaggtgggtcggatgaggctctgaactccggaatcctgactattgacgtcacttctgtgagctcctctctgggagggaacctccctgctaataatagctccctagggccgatggaacccctggtcctggtggcccacagtgatattcccccaagcctggacagccctctggttctcgggacagcagccacggttctgcagcagggcagcttcagtgtggatgacgtgcagactgtgagtgcaggagcattaggctgtctggtggctctgcccatgaagaacttgagtgacgacccactggctttgacctccaatagtaacttagcagcacatatcaccacaccgacctcttcgagcaccccccgagaaaatgccagtgtcccggaactgctggctccaatcaaggtggagccggactcgccttctcgcccaggagcagttgggcagcaggaaggaagccatgggctgccccagtccacgttgcccagtccagcagagcagcacggtgcccaggacacagagctcagtgcaggcactggcaacttctatttggtatgaagcactctattcagtcaccaccatataggtcacttctctcatactcggtcttgaggatattctggattaatcctttctatgcagacgtttctggtttacaaaaggacgcagccctggactacaagtctggaactgacaagttcttatgaccttgacaaatcaccttaacccatctgagccttaaattctcatttatttcctgcataaggagatttggctaaatgctttctgaggtcctttggagtcctgtggctccatggtaatgtgctcctttccttgaagattgggggttttgtaatgttgagatactttgcctctatgcttgtcagctcatgaccagtcctagaagaggagtcgagacataagccaccttcagaggttcaatggaaactttaaaaccataccaaactcttttttaaaattagaattaacaagaaaaaaaaaaagggtggggtttatgagccttagttcttggaggattataagagtacttccccagttttgaggctggacagttaatatactttatatcaattatacatttaatataatttaatttaaaataatttaaagattcttaggagatagtctgactttcctgacctagatgggaatgatcagatagggattttttttgtggcacaggctaaatttgatggtgacatttatattgttgagaatgttacatcttattttaccacaacttttaaaaaatgttacatcttttgcagtaggatcagttgtgaggcacatagtagctgaggctccatggagccacctttcatttctttcagtcagagaggaggacagtctctgtctctgcatttctggtgtcttgcttgtcggtggcagagccatgcttgccggcatttgcttaggcggccatagtagttgctaagtgtacaggtgactgggcagggatgggaggtggccacaggtcagagacaagtgctcagtcagtccctggtgccaggactgtgtgcctcggtgccttgggaaatggaagctccctggtgcagctgcagctgtgggtggaggtagagaagccagcaagaccttggtcttaaccccgtgttcattttcttgctagctgtgtgacgttgggctacctcgcttctctgagtacaaatggtgtgtggtgaatgggtcccaggtatgctacgagctttgagggctgctctttttctcttcatagcgataagtgttaaactgtctttcttaggaaacgttcacagacttgcaacagctgatgtcctctgagtactgtctgactccctcaggcaagttcctgaattcagtaccatcattattatttttgtgtaagactttgacaaagtatagcccctgccaccagagcagcctgtacagtgggtctctaaggtgggacctgccccgggcctgccatgcacgtgtgtgaaacagcgtgaaaagtgtcgcggtaaggtgaccctgggttacccaggcaaggctcggtgtttgtttcagaaagcagagaagtatgtaattgattttaaaagtttctgtttaaaatatttggctatgttttagactatgaaggaatgaactttgcttctctggataagaaagtcacatacattgttccagctccaagtttgttcggccctcgccacaagtggatgtagcgtttggccctttgtgtgccttgctggtgactctggttttgggagctcggatatgtcccagaagcaggcttatggcacttctgtagctcccttgctacccttcctttgtgtctagataagtgactgacatgcttttctttggtctcaggaaagtgggggctcagcaagaactgattaccgagccattcaactagccaaggaaaaaaagcagagaggagcggggagcaatgcaggtgaggccgtgtgtgctgcagccggacgagcaagggcctgagggttctctgtcactgttactggcagaagaaacacagcaggtgtttctgtgctcttggttttacttttctgttcagaatacccttttatcaactccttagttttatttgaacttaagggaaaaaattagtaacaaaattcccagcatcagtatgaacatattttatttgcctaaacaagctttgtgaaagttaagcgttcaaacaccagtgtcagttacctggaaggctactaaggtaaataagcaaagcaggccagttgtcaggaaagcagagattgtgcctggtgctgaatggccttggggcctgatcttggcatggcagagacctggggactgccactgtccccaggtacgtgtacatggagccaaactgtgtgtcctgtggcattgtcagagttatgttgaaatcttatttgaaaatgttagcaacttacttgcatttttaaagaccaaacaagagctggtaacctatggcctcaagcatctgtccttcctaaaaatggaatagtgggatgtagtgcttaatggaaactgctaaatctttttctaaaaactaacagtggatttttaaaatatattgttttttgtgtatttcatttgtcctttgtatttatctaaaagggttgatatgattttatatcttgctctctattcctaatagtattatgacttcttatttaaaataaataacaattgccggttttctgttaaaaaaaaaaaaa

SEQ ID NO: 37 - RNA, 2817 nt, Homo sapiens
  source 1..2817: mol_type = mRNA
agtcggcggccggactgggaagatggacgcagctactctgacctacgacactctccggtttgctgagtttgaagattttcctgagacctcagagcccgtttggatactgggtagaaaatacagcattttcacagaaaaggacgagatcttgtctgatgtggcatctagactttggtttacatacaggaaaaactttccagccattggggggacaggccccacctcggacacaggctggggctgcatgctgcggtgtggacagatgatctttgcccaagccctggtgtgccggcacctaggccgagattggaggtggacacaaaggaagaggcagccagacagctacttcagcgtcctcaacgcattcatcgacaggaaggacagttactactccattcaccagatagcgcaaatgggagttggcgaaggcaagtccataggccagtggtacgggcccaacactgtcgcccaggtcctgaagaagcttgctgtcttcgatacgtggagctccttggcggtccacattgcaatggacaacactgttgtgatggaggaaatcagaaggttgtgcaggaccagcgttccctgtgcaggcgccactgcgtttcctgcagattccgaccggcactgcaacggattccctgccggagctgaggtcaccaacaggccgtcgccatggagacccctggtacttctcattcccctgcgcctggggctcacggacatcaacgaggcctacgtggagacgctgaagcactgcttcatgatgccccagtccctgggcgtcatcggagggaagcccaacagcgcccactacttcatcggctacgttggtgaggagctcatctacctggacccccacaccacgcagccagccgtggagcccactgatggctgcttcatcccggacgagagcttccactgccagcacccgccgtgccgcatgagcatcgcggagcttgacccgtccatcgctgtggggtttttctgtaagactgaagatgacttcaatgattggtgccagcaagtcaaaaagctgtctctgcttggaggtgccctgcccatgtttgagctggtggagctgcagccttcacatctggcctgccccgacgtcctgaacctgtccctaggtgagagctgccaagtccagattcttctgatgtagagcgactggaaagattcttcgactcagaagatgaagactttgaaatcctgtccctttgaaaatcctggggtcgggggtggcacctgtgagagcctggggctcctggtgccgctgcgtttcatccatcccgcccgctcgcctgccgagggctgcgccccgtgctgcctccccccagagggccacccgctgtgctcgtggactgaggctgcgctgcccgggaggccttactgcttggtgtcagactgcccagctcagagtgcccgtcagggcctgtgcatccgcacgcggagccgtctgttaggagcttccagagtgttctctcgacactgccagccccgtgttagcacctgggcctcagtcccacttgctcccaggcgccggttctgtggttggtttggaattaaagtcctgtttgaagttgtcagacacagacatgaatttctgggcgctccctgagtcagagtctcagaagacctgtgcaggctggcgtgagaggagcggcagccacactgcggccccacgcccaaggactgggctgctctcgaggggggcgcgcccaccgctgtgtcctctctgcccagcctggcttaccaagggctacctcagtgggagatgaggttggaggaacgaaggcgaggttcctccttgctttggggagaaaagtattcaggaagtgggtgtgtgggaaacctgaagatggcgtgcacaggacacagcgtgggcggcctgggcagaagggcggctggctgtcctggagctgctgctggagcctgccctcagagtgtccctttccagtgctgtggcattctgtggcagcttccccaggtgtggtgacggggggggggcggggcctccacctgtgacagccaggcttgagggtggacggcgtgcctctcccaggagccttccccatgtccttgccttgctgagaattgccctcccatgccgctgaggtgttaggtggtttagggccaaaaggggaaaaccacttgagtcttgtggtgtgtggtgggcagacaccacagggtggcatcacctggtggcatttccagaacctcagccccgattccagcacccaccaccgcctgaccctgtgtaacctgctgtcccgggtcccagagtgcactctgccccgctgctctgctgcctgtcctgggaaagtatctttgccccactaggaaatgtaaacaggagggcttggggagcgtgggcacttttctcatgagcagctactgcggcgttggcaggactcgctgctgctgctgctgcttgtgtaggtcggggagccagagatccccgaggacgcgcgccggacagtcggcactgaccggcccacctggtagcagaggacacccccagccccccaagcattgaagacatagtgtatttcctcgtatcctttctcccttgggtgtagttggggtggggaagcagggaaggctggtgcgatctccattccttgggctccacgtccgagttcatggtgcgccgctgtgctgggagctgcagtggtaatgtgtgggacaccttgaccaaaggggagctttgtctcgtgtgttttgaaaaaggcttaatgaagagaatgttgttcattcttagtagtatagtttgcaattcttaatggcaaataataagtttcagtagaaaacaaa

SEQ ID NO: 38 - RNA, 1900 nt, Homo sapiens
  source 1..1900: mol_type = mRNA
gctctctcgcgctaccctcccgccgcccgcggtcctccgtcggttctctcgttagtccacggtctggtcttcagctacccgccttcgtctccgagtttgcgactcgcggaccggcgtccccggcgcgaagaggctggactcggattcgttgcctgagagatggtgtcttgctatgttgcccagacagatcttgtactcctgggctcaagttgtcctcctgcctcagcctcccaaagtgctgggattaaagatcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcattttttaaattcccgttttgtcactttttctgataaaagatggccatattaccccttttcggccccatgtatctcagtaccccatggagctgggctaagtaaataggaattggtttcacgcctgaggcaattagacactttggaagatggcataacctgtctcacctggacttaagcatctggctctaattcacagtgctcttttctcctcactgtatccaggttccctcccagaggagccaccagttctcatgggtggcactcagtctctcttctctccagctgactaaactttttttctgtaccagttaatttttccaactactaatagaataaaggcagttttctaaa

SEQ ID NO: 39 - RNA, 4445 nt, Homo sapiens
  source 1..4445: mol_type = mRNA
cttgcggaagtgacggcagttccgagtccagtgggggcggtgggagcgatgagggtctgagacggtgggagcggttgtgtgaagatggagacattccatacaccaagcttgggtgatgaggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttcccagccagaagacagtcgtggtggaagcagggaaaaagcagaaggccccaaagaagagaaaaaagaaagatcctaatgaacctcagaaaccagtttcagcatatgctttattctttcgtgatacacaggctgccatcaagggacagaatcctaatgccacttttggtgaggtttcaaaaattgtggcctccatgtgggatagtcttggagaggagcaaaaacaggtatataagaggaaaactgaggctgccaagaaagagtatctgaaggcactggctgcttacaaagacaaccaggagtgtcaggccactgtggaaacagtggaattggatccagcaccaccatcacaaactccttctccacctcctatggctactgttgacccagcatctccagcaccagcttcaatagagccccctgccctgtccccatccattgttgttaactccaccctttcatcctatgtggcaaaccaggcatcttctggagctgggggtcagcccaatatcaccaagttgattattaccaaacaaatgttgccctcttctattactatgtctcaaggagggatggttactgttatcccagccacagtggtgacctcccgggggctccaactaggccaaaccagtacagctactatccagcccagtcaacaagcccagattgtcactcggtcagtgttgcaggcagcagcagctgctgctgctgctgcttctatgcaactgcctccaccccgactacagccccctccattacaacagatgccacagcccccgactcagcagcaagttaccattctgcagcagcctcctccactccaggccatgcaacagcctccacctcagaaagttcgaatcaatttacagcaacagcctcctcctctgcagatcaagagtgtgcctctacccactttgaaaatgcagactaccttagtcccaccaactgtggaaagtagtcctgagcggcctatgaacaacagccctgaggcccatacagtggaggcaccttctcctgagactatctgtgagatgatcacagatgtagttcctgaggttgagtctccttctcagatggatgttgaattggtgagtgggtctcctgtggcactctcaccccagcctcgatgtgtgaggtctggttgtgagaaccctcccattgtgagtaaggactgggacaatgaatactgcagcaatgagtgtgtggtgaagcactgcagggatgtattcttggcctgggtagcctctagaaattcaaacacagtggtgtttgtgaaatagtccttcctgttctccaagccagtgaagagttatctgctgggaaagtgtccaagagcctgtttttgaaacacaagctgggcttctggtagtgcctcatcacaacccatgatggctgttcatgtttcaccccttttcttccttcagcagaggccaggctatggagcagggccactgaatttgctgtaatctggagatgctttttactttcaaccataagcggtaatagcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgtgttttttgttttttttttttttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagtta

SEQ ID NO: 40 - RNA, 4713 nt, Homo sapiens
  source 1..4713: mol_type = mRNA
cttttccgcccgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgatgcaaaagaaagaaatccctgcgctttctgtctgtctttgtggcggcccagattgaattggggaatacatctttagcctggaaatgtaggctgcatgttaatggtaatgtaacttttgcagtgtaatgtttgaaaaatattaatgtagtttttgcttttacagtaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagagaatgccttttagtttaatgcatatttaaactaaattgatcctgtagtgttcctggagaagctagagcctgattgtaggctactactcatcaattaacttctacagtggagactacttctgggactggaatataaaaaagaatcaaaggttctgattttgagttgcaataaagggaaagaccatgctcatagcagtgccaacatctgaagtgtggagccttacccatttcatcacctacaacggaagtagttaactggaagagattaccaagagaataaaaagagactcattcagtggaagcaactttgtctcagcttatttcacataaagagagcgaagtcttttgggatgaatgttaattaaactccctggtaactagaacagggactggcaaactagcctatctgaccacctgttttgtacactttaaggtggttggttgcctttttaaatggttgaggggaaaagaataccttgtgggatatggaatttaagttcgagtccagttttattggaacgtggctatgcttattcatttatggattgactgtggctgttgtcagtgcatgagcagagttgtgtctaacagactagagcctgcaagtttgccagcccctgatttaaaagatgaaggtacacagaatgtgggctggctggtgggcaaaggggtaaaaatgttctctatattgtatctgaaaagatggggtgtctgaataagaaaatgcatctatttgacagacctggagcagttgctatctgctgctatggtttccaccacagatgcaagaagaacatgtccttgcgctttccgtctgtctaattgtggcagctgagattgaatagaggaatacaggaggaaaaaaagcgggaagagtttttgaggcaggtcggtcacccaggcttgtagtgcagtggcacaagcaactcactgcattctctgcatcctgtgctcaagccattttcccacctcagtctcactagttgctgggactgcaggcatgcacccctatgcccagctaatttttgtagagaccgagtatcgcttagttgcccagggtggtctcaactcctgggctcaaggagatctgcccacctcagcctcccaaagtgcaggcctagcctgggaggggaattttcaaaacgtgagttttgggaaatagtctatcagccttacctggttgattacacttgtaaaagaaagattaaaagcaggccagtgactctggtctgcttgaacatgtgaatgtagtggtttgagcaatctggagtttgccctagtgtcaaattccagactgtccatagtgtccaaaacctgaggcagatactaatgttaacccccagcaccccgtgattggaaacaaacctaaatacgtattgggaacttaatagcaattttaagcattctgatagattttttgtagggatggggtcatgccatgtggcccaggctggtctgaaaactctggcctcaagtgatctcaagctttggccttctaaagtgttgggattacaggtgtgaggcattgcacctggcttagcgttctgatttgacattgtaatgaaaagtgtgagtctcatctacagggccttttgtcctctgaaatgatagcaggaagggaattttcaggcagtggtcaaagctggggaaaccaggatagtgaagaaggccttgaggtgagagatggaagctaattggtgaactagccttggaagcctgaaacagacaagtagcaattcagagactttgtgggctccactgctccaacttgttttgaagattttcagttctgcagaagaggtatttccccagttgtcctttcagtgctcttagctgttttcccaacatccagatccaatcaaggctgggacatagcattttatcatgtctatttaagtcagaagtgatgaaccccagctgtttacctcatggtaaacctttgaagattccaggtagaatcttctcagactttgaagactgtctcattttatatctttttctcgttattcctagggtcaagacgttttgggcaagaataaggatgtgaacatcagaaagctcataacattttgtttttgatgctaagtttaacaaaggcatgctttagtagcctgtgggccctagggtttgttaaagtgtggagaacaactgagtggagcaagaggacttttctaggaaggtccttgtaatgtgacatttgaaaacaaatgaaggtgtggaagtaggccatgtggatatcaggacaaaccattccaggccaagacaacagcagttagtctggagtgtgatgtgttctgggaaaaaagtggccactttgctaacccaagaagacaggaagggttgtaaagcagtgggagtgtgcaaggaaggaagaccagacctcaaggaaaccacaggcgctctgagcagaagagttacatgatatgactcaaatttttaaaggatcactttggctgccaggtggcagggtaaaagcatagaataattgtgtataatgtgtttttaaggcaaagatagtggcttagtctagggtagtagactgaggtggtaggaaatgaagatagagacaacaggatatgctggtgggtgaggatggatttaatgttgatacaagtattttggtctgagcgtttggaagaaagttggcactgaggtgggaagtcgagtttagttttgttagttttggatgtgttaagtttgagatgctgattcttcagagaagtctaagctggagaactatatagagagtggaaagataacaatagacattgaaagccatgatacaggataaggtcatttggagagaggatagactgcattccaacatgagattggttgacaaagagaaaccaacaaaggtaattaagaggtgctcccactgcacttgtactcagaaggctgaggtaggattgttagaggccagcctgggcaccacagggagaccccatctctaaaatttagccaggaaccatggctcatgcctgtagccccaggaatttgggaggctgagtggggaggatcgcttgaggtcaggagtttgagaccagcctgggcaacatagggagacctaaaaaaattaattgggcatctgtagtcccagctactcaggcggctgagctgagaggatggcttgagtccgagagattgagggtgcagtgagctgtgatcataccactgcactccagcctgggcggcagtgagacactatctgaaaaaagtttaaaaattttaaaaaagaaggaactgcccctgaggtaagaaccaagggagggcctcccagaggtcaggtggaaaaagttttaggaaggaggaagtagtcaacagggttacctgttgcaaagtacttaagtaatatgaggcctgatagtggtaaacttgactaccgttggatttcactagtgggaaaggaagtctaattaaaatgcactcaagagactaacagtcgcaggcatgaaatacaatacaggtacatggttttttattatgtgtgcatctgcttcagtaataggtgtgaattactcatttggatcattaggagtttcaaaatctagttaaatgactagatttttgttgatgtaaattctgtcattctgaactgcagggattgtcagtaacttaactgcaaactaaactggtgataattatggtaaaattgcaagacgagcaataaatctcaaccaacttgagagaacactgataa

SEQ ID NO: 41 - RNA, 2610 nt, Homo sapiens
  source 1..2610: mol_type = mRNA
attctttgtctacttcctatttgagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaa